(19) United States
(12) Patent Application Publication
(10) Pub. No.: US 2008/0233090 A1
(43) Pub. Date: Sep. 25, 2008

(54) Method of Treatment by Administration of Rna

(76) Inventor: Stephen Ray, Oxfordshire (GB)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/814,272

(22) PCT Filed: Jan. 19, 2006
(86) PCT No.: PCT/GB2006/000186

§ 371 Date: May 27, 2008

(30) Foreign Application Priority Data
Jan. 19, 2005 (GB) 0501129.1

(51) Int. Cl. A61K 35/12; A61K 31/70; A61P 43/00

(52) U.S. Cl. 424/93.21; 514/44; 424/93.7

(57) ABSTRACT

A method of effecting a treatment response in a target tissue of a subject, comprising: administering to the subject isolated RNA comprising an RNA sequence extractable or extracted from a source tissue such that said treatment response is effected; wherein the RNA is isolated polyA positive RNA in substantially pure form.

All documents cited herein are incorporated by reference in their entirety.

### FIELD OF THE INVENTION

The present invention relates to the alteration of cell properties. In particular, it relates to the alteration of one or more of the capacities of a cell to mobilise, migrate, integrate, proliferate and differentiate, where such capacity is latent or evident, and where each capacity may be manifested in any order. For example, it relates to the alteration of properties of stem cells, including the acquisition of the evident or latent capacity to mobilise, migrate, integrate, proliferate and differentiate. It also relates to the in vivo alteration of stem cell properties, including the acquisition of the evident or latent capacity to mobilise, migrate, integrate, proliferate and differentiate. It also relates to the in vitro alteration of the stem cell properties, including the acquisition of the capacity to mobilise, migrate, integrate, proliferate and differentiate, where such alterations of property may be evident or latent in vitro, or may only become evident after subsequent introduction to a host in vivo, or after subsequent introduction into a further phase of in vitro culture, including introduction into an ex vivo preparation. Accordingly, it relates to the promotion of functional repair and or regeneration. The invention further relates to the induction of differentiation of stem cells.

### BACKGROUND OF THE INVENTION

### Tissue-Derived RNA

RNA extracts have been shown to affect the differentiation of tissues, for example when applied to chick embryos (Sanyal et al (1966) PNAS, 55:743-750), mouse ascite cells (Niu et al (1961) PNAS, 47:1689-1700) and mouse uteri (Yang et al (1977) PNAS 74:1894-1898). They have also been shown to affect the properties of neoplastic cells, for example in rat hepatoma cells (DeCarvalho et al (1961) Nature, 189:815-817) and leukaemic patients (DeCarvalho et al (1963) Nature 197:1077-1079). Moreover, RNA extracts have been shown to have effects in the immune system, for example in the transfer of immune properties from donor to recipient (Rascati et al (1981) Intervirology 15:87-96 and DeLuca et al (2001) Molecular and Cellular Biochemistry 228:9-14).

The present invention is based on the discovery that RNA derived from specific tissues of the human body can be used to repair, regenerate or enhance target tissues in a subject.

### Engraftment of Genetic Modifications

Methods currently exist, and new methods may in time be developed, for the specific modification of the genotype (e.g. to correct a genetic fault) of a small fraction of cells in vitro. For example, small fragment homologous replacement (SFHR) can be used to correct the cystic fibrosis transmembrane conductance regulator gene in a small percentage of cells in vitro. Similar techniques can also be used in vivo, although transformation efficiency is even smaller.

Accordingly, there is currently no effective method for the transformation of a significant percentage of cells in vivo.

Although techniques for in vivo transformation may be improved, the transformation efficiency is likely to remain substantially lower than that obtainable in vitro. Moreover, the transformation frequency for in vivo transformation is likely to remain below a clinically useful level.

However, despite the limitations of current in vitro transformation techniques, it is possible to create genetically modified stem cells in vitro and expand their number by in vitro propagation. Accordingly, it has been suggested that a population of genetically modified bone marrow stem cells could be used to repopulate the bone-marrow of a mammal (Hacein-Bey-Abina et al (2002) NEJM, 346:1185-1193. Once bone marrow has been replaced in this way, other haematopoietic cells may be replaced with genetically modified counterparts derived from the genetically modified bone marrow stem cells. This approach therefore provides a means for altering the genotype of hematopoietic cells in a subject.

However, there remains a need for a technique for modifying the genotype of cells other than bone marrow/haematopoietic cells. A generally applicable method that allows such modification would be useful in a wide variety of clinical situations. For example, in Duchenne muscular dystrophy, there is a need is to modify the genotype of muscle cells.

The present invention therefore seeks to provide a generally applicable method for modifying the genotype of cells in a subject.

The present invention solves this problem through the use of RNA derived from specific tissues of the human body. For example, the application of RNA of the invention, with or without tissue ablation or resection, can facilitate the replacement of autologous tissues with their genetically modified counterparts, even in non-haematopoietic tissue. In particular, the use of RNA of the invention provides a method for promoting the migration of stem cells administered as a part of the method to any other tissue. Once migrated to a target tissue, the stem cells can integrate, proliferate and differentiate in the tissue, resulting in tissue regeneration or growth. Accordingly, in a specific embodiment, the present invention provides a method for enhanced regeneration of tissues after stress and partial/complete removal by ablation or resection.

### SUMMARY OF INVENTION

The present invention is concerned with the use of RNA to alter of cell properties. In particular, it relates to the use of RNA to alter differentiation and the ability of cells to mobilise, migrate, integrate, proliferate and differentiate.

Without wishing to be bound by theory, the present invention is based on a hypothesis presented by the present inventors in co-pending international patent application PCT/GB2004/002981 that the behaviour of cells used for tissue regeneration is governed by the transfer of information via RNA from tissues in need of regeneration to effector cells.

More specifically, the present inventors propose that the normal physical repair, renewal and regeneration of an organ is mediated in part by signals derived from the tissues that make up the organ. These signals cause the activation, migration, mobilisation, integration and proliferation of both tissue-specific and non-tissue-specific stem cells required for the physical repair, renewal and regeneration of the tissue. However, age and/or disease impair the normal physical repair, renewal and regeneration of a target tissue.

The present invention is based on the discovery that administration of RNA can provide or restore the signals that cause the activation, migration, mobilisation, integration and proliferation of both tissue-specific and non-tissue-specific stem cells required for physical repair, renewal and regeneration of the target tissue. Moreover, the present invention is based on the further discovery that RNA administered to stem cells prior to their administration to a subject improves their ability to repair, regenerate or enhance a tissue.

The ability to enhance the migration of stem cells into tissues and to promote the generation of local tissues provides a means for promoting the transfer of a genetic modification from stem cells to peripheral tissues. Moreover, the ability to promote the growth of new tissue provides a means of treating a variety of disorders.

Like the invention disclosed in PCT/GB2004/002981, the present invention is concerned with the promotion of stem cell-mediated functional repair, the treatment of various disease conditions by influencing mobilisation, migration, integration, proliferation and differentiation of cells, the differentiation of stem cells in general and their acquisition of the ability to mobilise, migrate, integrate, proliferate and differentiate. The present inventors have found that it is possible to induce stem cells to differentiate into a desired differentiated cell type and that it is possible to induce stem cells to mobilise, migrate, integrate, proliferate and differentiate into a desired differentiated cell type which is integrated into a targeted tissue. This is achieved by providing specific RNA sequences to the target cells.

The ability to influence cell fate allows a variety of clinically useful phenomena to be induced including allowing diseased cells, tissue and organs to be repaired, allowing specific cell types and cell fates to be induced, and so on. The ability to induce stem cell mobilisation, migration, integration, proliferation and differentiation in vivo means that stem cell-mediated functional repair may be beneficially promoted in intact organisms, and particularly humans. PCT/GB2004/002981 describes a method for altering a cell property towards a property of one or more desired cell types comprising providing isolated RNA comprising a RNA sequence extractable from cells comprising said desired cell type(s) to a population of cells under conditions whereby the alteration of the cell property of said cells is achieved.

The isolated RNA may be extractable from or extracted from one or more cell types that possess the property or properties of interest. The isolated RNA may comprise the sequence of RNA extractable from one or more cell types that possess the property or properties of interest. It is thus not always necessary to extract RNA from the desired cell types; the RNA sequence conferring the advantageous property or properties onto the cell type may be generated synthetically, for example, using a recombinant expression system. Larger quantities of the desired RNA may be produced by the in vitro expansion of isolated RNA.

The population of cells may be exposed to the RNA in vitro, or in vivo. In vitro, the population of cells may for example be a cell culture, such as in a cell culture dish or roller bottle or cells growing on a support, membrane, implant, stent or matrix; or a tissue, such as an isolated tissue grown outside the body. In vivo, the population of cells may be a human patient, or a tissue isolated from an organism, such as an organ, a specific part of an organ, or a specific cell type or collection of cell types.

The present invention encompasses and adds further embodiments to this invention as described in PCT/GB2004/00298.

In one aspect, the present invention provides a method of effecting a treatment response in a target tissue of a subject, comprising administering to the subject isolated RNA comprising an RNA sequence extractable from a source tissue such that said treatment response is effected. The RNA may be extractable or extracted from the source tissue.

In another aspect, the invention provides a method of effecting a treatment response in a target tissue of a subject, comprising administering to the subject stem cells such that said treatment response is effected, wherein said stem cells have been treated with isolated RNA comprising an RNA sequence extractable from a source tissue.

In some aspects, the methods of the present invention may be used to improve stem cell-mediated repair, either in vivo or in vitro. In one embodiment, this aspect of the present invention provides a method of inducing totipotent, pluripotent or unipotent stem cells, preferably derived from a tissue of the subject to be treated, or of a stem cell line, again preferably derived from a tissue of the subject to be treated, to differentiate into one or more desired cell types, which comprises providing isolated RNA comprising RNA extractable from tissue or cells comprising said desired cell type(s) to a cell culture of said stem cells under conditions whereby the desired differentiation of said stem cells is achieved. The cells generated in vitro in this manner may then be delivered into a recipient. For in vivo treatment, the stem cells may reside and be exposed to the RNA in situ, in the body of the subject. Alternatively, the stem cells, with or without pre-treatment as above, may be administered to a tissue or an organism on their own or in conjunction with RNA according to the invention, to induce mobilisation, migration, integration, proliferation and differentiation of stem cells in vivo in the treated subject. Cells and RNA may also be administered in simultaneous, separate or sequential application with other therapies effective in treating a particular disease. In one embodiment, RNA extractable from one or more stem cell types or stem cell active tissue(s) may be administered in simultaneous, separate or sequential application optionally with cells, such as stem cells. For example, in specific preferred embodiments, RNA from an embryo or fetus, from the whole body, an organ, a specific part of an organ, or a specific cell type or collection of cell types is administered in simultaneous, separate or sequential application with stem cells, or cells derived from in vitro treatment of stem cells, particularly bone marrow stem cells.

In some cases the isolated RNA itself may be used to induce differentiation in situ. Similarly, the isolated RNA itself may be used to induce mobilisation, migration, integration, proliferation and differentiation in situ. Thus in another aspect the invention also provides for the use of the RNA capable of inducing differentiation of cells, particularly stem cells, in the treatment of, or in the manufacture of a medicament for use in improving or rectifying, tissue or cellular damage or a genetic disease. The present invention also provides for the use of the RNA capable of inducing migration, mobilisation, integration, proliferation and differentiation of cells, particularly stem cells, in the treatment of, or in the manufacture of a medicament for use in improving or rectifying, tissue or cellular damage or a genetic disease, including repair of diseased cells and induction of specific cell types and cell fates. The isolated RNA may be provided to the cell population as a medicament in which the RNA forms the principal active ingredient of the medicament.

The isolated RNA may be used to induce mobilisation, migration, integration, proliferation and differentiation of stem cells in vivo. Accordingly, in another aspect the invention provides a method of treatment comprising administration of the RNA capable of inducing differentiation of stem cells in a therapeutically effective amount to a subject in need thereof. The invention also provides a method of treatment comprising administration of the RNA capable of inducing mobilisation, migration, integration, proliferation and differentiation of stem cells in a therapeutically effective amount to a subject in need thereof. Such methods may be used, for example, to promote stem cell-mediated functional repair, including repair of diseased cells and induction of specific cell types and cell fates.

Isolated RNA comprising RNA extractable from particular desired type(s) of stem cell or stem cell line may thus be used to promote stem cell-mediated functional repair in vivo, and to improve or rectify tissue or cellular damage or a genetic disease. Such damage may be due to, for example, disease, age or genetic makeup or genetic mutation, trauma, surgery, any other form of treatment, disease, or accidental or intentional morbidity.

Alternatively, the RNA may be applied to the cell population in conjunction with other active agents, including, for example, stem cells, or cells derived in vitro from stem cells according to a method as described above. The RNA and other active agents may be administered simultaneously, sequentially or separately.

The method of the invention may be used to induce stem cells to mobilise, migrate, integrate, proliferate and/or differentiate into one or more desired cell types. This aspect of the present invention provides a method of inducing totipotent, pluripotent or unipotent stem cells, preferably derived from a tissue of the subject to be treated, or of a stem cell line, again preferably derived from a tissue of the subject to be treated, to migrate, integrate, proliferate and/or differentiate into one or more desired cell types, which comprises providing isolated RNA comprising RNA extractable from tissue or cells comprising said desired cell type(s) to a cell culture of said stem cells under conditions whereby the desired mobilisation, migration, integration, proliferation and/or differentiation of said stem cells is achieved. In some embodiments, the totipotent, pluripotent or unipotent stem cells are treated in vitro. In other embodiments, the totipotent, pluripotent or unipotent stem cells are treated in vivo, in the body of the subject. The stem cells employed may be adult stem cells.

The invention also provides cells obtained by the above methods. Such differentiated cells may be used in the manufacture of medicaments for treating a number of disorders. Thus, in a further aspect the invention provides for the use of the cells in the manufacture of a medicament for use in improving or rectifying tissue or cellular damage or degeneration or a genetic disease. The invention includes methods of treatment that comprise administration of these cells in a therapeutically effective amount to a subject in need thereof.

### BRIEF DESCRIPTION OF THE DRAWINGS

FIG. 1: The effects of brain RNA differentiated stem cells on age-related damage to the rat brain assessed by spatial learning and memory performance of recipient animals. Ex-breeder male rats aged between 468 to 506 days were given intravenously either untreated bone marrow stem cells or bone marrow stem cells treated with brain RNA extract. The results for control rats that received untreated stem cells (closed boxes) and those for experimental rats that received brain treated stem cells (open circles) are shown. The results show a remarkable increase in learning ability in the experimental rats.

FIG. 2: The effects of spine RNA differentiated stem cells on an animal model of motor neurone disease. SOD 1 mice were given intravenously either bone marrow stem cells treated with spine RNA extract, untreated bone marrow stem cells or physiological saline. The results for experimental mice that received spine RNA-treated stem cells (closed boxes), control mice that received untreated stem cells (open triangles) and control mice that received physiological saline (closed circles) are shown. The results show that pre-treatment of stem cells with spine derived RNA dramatically improved the efficacy of stem cell treatment in an established model of progressive neurodegenerative disease.

FIG. 3: The influence of donor tissue developmental stage on the effect of brain RNA differentiated stem cells on age related damage to the mouse brain assessed by spatial learning and memory performance of recipient animals. 254-299 day old C57/B1 mice were given intravenously either bone marrow stem cells treated with foetal (E15) brain RNA extract, bone marrow stem cells treated with adult (90 day) brain RNA extract or untreated bone marrow stem cells. The results for control mice that received untreated stem cells (closed boxes), experimental mice that received foetal brain treated stem cells (closed circles) and experimental mice that received adult brain treated stem cells (open triangles) are shown. The results show an increase in learning ability in the experimental mice, with the mice that received foetal brain treated stem cells demonstrating significantly faster learning.

FIG. 4: The effects of direct injection of bone marrow stem cell derived RNA on age related damage to the rat brain assessed by spatial learning and memory performance of recipient animals. Ex-breeder male rats aged between 433 to 570 days were given injections of either bone marrow stem cell RNA or bone marrow stem cell RNA treated with RNaze into the right lateral ventricle. The results for control rats that received RNaze treated stem cell RNA (closed boxes) and those for experimental rats that received stem cell RNA (open circles) are shown. The results show that control rats could not learn the task, while the stem cell RNA treated animals could learn the task with comparable performance to young rats.

### DETAILED DESCRIPTION

### Alteration of Cell Properties

The inventors have found that provision of RNA sequences from particular sources to cells can influence cell properties. In particular, the present invention is in one aspect concerned with the promotion of stem cell-mediated functional repair. By “repair” is meant restoration, regeneration, strengthening, renewal, rejuvenation, or partial or complete regrowth or renewal of a tissue. Stem cell-mediated repair may occur either in vitro or in vivo. The invention is also concerned with the differentiation of stem cells to adult, specialised cells. This is achieved by providing specific RNA sequences to the target cells.

The present invention is generally concerned with the alteration of a cell property. By “property” is meant any desired property of a cell, including a biological property that is reflective of the type of biological molecule(s) that is present in or on the surface of, or secreted by, a cell. A desired property includes the active state of a particular biological molecule(s) in the cell, or the capability possessed by a cell for a particular behaviour.

The property may be a latent property or may be evident in the cell. The property may be a particular phenotype, by which is meant any observable physical or biochemical characteristic. Such a phenotype change may be, for example, the expression of a cell surface marker, altered immune function, altered MHC restriction, altered activity of one or more proteins, and so on. A desired phenotypic change may be more extreme e.g. redirection of cell function from one tissue to another, such as a liver cell towards a kidney cell. Such a phenotypic change may be reversal of tumour cell activity toward healthy cell activity.

The property may thus be any desired function that is possessed by a cell. The term “function” is meant to include any biological activity that is effected by the desired cell type. Examples of functions include those that are specific to a particular tissue, for example, brain (for example, cortex, cerebellum, hippocampus, retina, substantia nigra, subventricular zone), spinal cord, liver, kidney, muscle, nerve tissue (peripheral, central, neuronal, glial), cardiac tissue (for example, atrial, ventricular, valve, cardiac innervation), immune cells, blood, pancreatic tissue, thymic tissue, spleen, skin, and gastrointestinal tract, lung, bone, cartilage, tendon, hair follicle, sense organ (for example, ear, eye), any gland either endocrine, exocrine, paracrine, such as thyroid, thymus, pituitary, adrenal, pancreatic, reproductive system (for example, testicular, prostate, seminal vesicle, ovarian, uterine, fallopian mammary), dental, vascular, digestive tract tissues (for example, stomach, gall bladder, intestines, colon). At a more detailed level, the function of particular cell types within a tissue type may be of interest, for example within brain tissue, neuronal cells or cortical neurones or glial cells have more specialised functions within the brain. At a more detailed level still, desired functions may be at a molecular level, where it is desired for specific molecules to be expressed on the surface of cells, such as specific T cell receptors in the case of T cells of the immune system. It is not possible for any list of desired function to be exhaustive and equivalent functions that may be desired in each circumstance will be apparent to the skilled reader.

The alteration of the property may result in the cell undergoing differentiation towards a more specialized form or function, for example from a stem cell towards an adult cell with a specialised function (for example, a hepatocyte). The alteration may also result in the cell and its progeny acquiring the behaviour of mobilisation, migration towards, and/or integration with, one or more tissues, organs or other sites, and proliferation. By “mobilisation” is meant change of stem cells from a quiescent resting state and, when in vivo, departure from their quiescent resting location. By “migration” is meant movement of stem cells from their point of mobilisation or artificial delivery, towards and into a target tissue. By “integration”, is meant the interaction of stem cells and their integration with target cell populations and their environment.

The alteration may also result in the cell and its progeny acquiring the behaviour of proliferation, prior to, during, or after migration and integration. By “proliferation”, is meant division of cells and their progeny to provide new tissue.

Accordingly, in one aspect the present invention provides a method of directing differentiation of stem cells towards one or more desired cell types comprising providing isolated RNA comprising an RNA sequence extractable from cells comprising said desired cell type(s) to a population of stem cells under conditions whereby the desired differentiation of stem cells in said population is achieved. The invention also provides a method of directing mobilisation, migration, integration, proliferation and differentiation of stem cells towards one or more desired cell types in vitro, or integrated with a target tissue in vivo, comprising providing isolated RNA comprising an RNA sequence extractable from cells comprising said desired cell type(s) to a population of stem cells under conditions whereby the desired differentiation of stem cells in said population is achieved. The RNA may be extractable or extracted from cells comprising said desired cell type(s).

This method allows the direction of differentiation to be dictated towards a particular speciality. For example, the stem cells may be directed towards liver function, or more specifically hepatocyte function. The method allows the mobilisation of a particular type of stem cell, for example bone marrow mesenchymal stem cells. The method allows for migration and integration into a particular tissue e.g. the left femur.

The invention provides methods and medicaments for the controlled manipulation of any cell, in particular stem cells, to induce the cell to differentiate into a desired differentiated cell type. Such methods include the improvement of stem cell-mediated repair, through directing the mobilisation, migration, integration, proliferation and differentiation of stem cells.

Such methods include the induction of stem cells to differentiate into one or more desired adult cell types.

A stem cell may, for example, be induced to differentiate in order to achieve a specific terminal differentiated state. The ability to choose what type of cell to induce the stem cell to differentiate into means that it is possible to produce a variety of different cell types from a single stem cell line or stem cell line. The RNA molecules of the invention, or differentiated cells types obtained, may be employed in treating, or in the manufacture of medicaments for treating, various disorders. In particular they may be used for improving or rectifying tissue or cellular damage or a genetic disease. The invention also provides methods and medicaments for the induction of in vivo stem cell mobilisation, migration, integration, proliferation and/or differentiation and the promotion of stem cell-mediated functional regeneration and/or repair.

The ability to influence cell fate using RNA allows diseased cells to be repaired and allows specific cell types and cell fates to be induced.

### Medicaments and Methods for Treating Subjects

The RNA and RNA-treated (e.g. differentiated) stem cells provided by the present invention may be used to treat a number of disorders, and in the manufacture of appropriate medicaments.

The subject of these methods, or the intended recipient of the medicaments, is a bird or mammal. In those cases where the subject is a bird, it is preferred that the bird is a poultry-bird or other agriculturally important bird. However, it is preferred that the subject is a mammal, and most preferably a human. In those cases where the subject is a non-human mammal, the mammal may be a domestic mammal or an agriculturally important mammal. The mammal may, for example, be a sheep, pig, cow, horse or bull or other commercially-farmed mammal. The mammal may be a dog or cat or other domestic mammal. The mammal may also be a non-human primate such as a monkey. For example, the primate may be a chimpanzee, gorilla, or orangutan. The mammal may also be rodent, for example a mouse, rat or hamster.

However, as noted above, the subject of these methods, or the intended recipient of the medicaments, is preferably a human patient. Preferably, the human patient is an adult, juvenile or baby. More preferably, the human patient is an adult or juvenile. Most preferably, the human patient is an adult. RNA extracts have been applied to embryonic tissue in certain studies (see, for example, Sanyal et al (1966) PNAS, 55:743-750). Accordingly, in some embodiments of the present invention, embryos are explicitly disclaimed from the subject of the methods and medicaments of the invention.

In preferred embodiments, the methods and medicaments of the invention are for effecting a “treatment response” in a target tissue of the subject in vivo. As used herein, a “treatment response” means an effect that is desired in a target tissue. Said effect may be defined as a change in one or more physical parameters of the target tissue. Such changes may include one or more of the following:

1) growth—the increase in size of the target tissue, e. g. of an organ or part of an organ, or the increase in thickness of a particular layer of a particular tissue;

2) regeneration—the restoration in presence, size and functional characteristics of the target tissue, where this target tissue has been degraded in these respects;

3) renewal—the restoration to a previous, more functional state of the target tissue, where this target tissue is present but has been degraded in these respects; and

4) repair—the rectification or reversal of physical damage to the target tissue.

The treatment response may be defined as any combination of the above in the target tissue. Accordingly, in preferred embodiments, the treatment response may be defined as any one or more of growth, regeneration, renewal or repair in the target tissue.

Accordingly, in some embodiments, the treatment response may involve the restoration or improvement in one or more functional parameters that are known or presumed to be associated with the target tissue. For example, in Alzheimer's disease the treatment response may be defined as the restoration of the ability to form long term memories and/or an improvement in the recall of old-established memories. In another example, for general disease- or age-related cognitive impairment, the treatment response may be defined as modification, repair, improvement or enhancement of language deficits, emotional and/or physiological control deficits. In other examples, the treatment response may be defined as improvement in cardiac performance (for instance, where there is heart damage) or improved renal clearance (for instance, in kidney disease).

In specific embodiments of the present invention, the treatment response may include the complete or partial gross regrowth of a resected organ (e. g. of a kidney or lung), or parts of organs (e. g. of one or more lobes of the liver).

Similarly, in other specific embodiments, the treatment response may include the gross regrowth of resected limbs or components of limbs, e.g. a digit, arm or leg.

As used herein, a “target tissue” means one or more tissues or one or more cell types in the subject in which it is desired to effect a treatment response. RNA extracts have been used in the treatment of specific cancers in certain studies. Accordingly, in some embodiments of the present invention, methods targeting tissue comprising cancerous (i.e. malignant) cells (e.g. tissue comprising one or more cells capable of forming a solid or non-solid cancer) are explicitly disclaimed from the methods and medicaments of the invention. Alternatively, in some embodiments of the present invention, methods targeting tissue comprising tumorigenic cells (e.g. tissue comprising one or more cells capable of forming a solid or non-solid tumour) are explicitly disclaimed from the methods and medicaments of the invention

The invention may employ a number of approaches to treat disorders, in particular to effect a treatment response, and to provide appropriate medicaments. In particular, administration of the medicaments of the invention to a subject to be treated may result in:
(a) administration of an RNA of the invention to a subject in order to induce differentiation of cells, such as stem cells, in situ; or administration of an RNA of the invention to a subject in order to induce mobilisation, migration, integration, proliferation and differentiation of cells, such as stem cells, in situ;
(b) treatment of stem cells with an RNA of the invention prior to administration of the RNA-treated stem cells to a subject;
(c) administration of an RNA of the invention to the subject prior to, in conjunction with or after administration of stem cells, which stem cells may or may not have been treated with an RNA of the invention; or
(d) any combination of a), b) and c).
   Generally, in the aspects of the invention under a) to d), in some cases it may be desired to use the methods of the invention to provide a cell type which is missing, depleted in number or functionally defective. The cells of the invention may be provided to a specific site or to a larger region. For example, the cells may be provided to a site of tissue or organ damage or injury such as a wound or broken bone. The cells may be provided to the site of a nerve injury and in particular to a spinal column injury. The cells may be provided to a damaged or diseased liver, kidney, heart or other organ. In the case of damaged or defective cardiac muscle disease such as in heart disease, dead or damaged cells can be augmented or replaced. Similarly cells can be provided to subjects with liver disease such as liver fibrosis, or other types of liver damage. Typically differentiated cells, or cells with altered properties (latent or evident) obtained using the methods of the invention will be provided, in some cases however stem cells obtained using the methods of the invention may be provided and allowed to differentiate in situ.

### a) RNA Therapy

It is shown herein that administration of RNA extracted from brain cells to a subject has the effect of stimulating resident stem cells in a subject to thicken brain cortex. Furthermore, RNA prepared from developmental stages known to show increased stem cell activity has been demonstrated to stimulate endogenous repair mechanisms. In one embodiment of methodology (a) above, the administration of an RNA in accordance with the invention to the subject induces differentiation of cells, such as stem cells, in situ in such a way as to promote stem cell-mediated functional repair. The administration may induce mobilisation, migration, integration, proliferation and differentiation of the cells in situ so as to promote stem cell-mediated functional repair. The administration may also induce a treatment response in a subject.

Accordingly, in one embodiment, this aspect of the invention provides a method of effecting a treatment response in a target tissue of a subject, comprising administering to the subject isolated RNA comprising an RNA sequence extractable from a source tissue such that said treatment response is effected. The RNA may be extractable or extracted from the source tissue.

This aspect of the invention also provides for the use of isolated RNA comprising an RNA sequence extractable from a source tissue in the manufacture of a medicament for effecting a treatment response in a target tissue of a subject. The RNA may be extractable or extracted from the source tissue.

The amount of RNA administered to the subject will be sufficient to bring about the desired treatment response. For example the concentration of RNA (e.g. in a medicament of the invention) may be from 10 ng to 5 mg/ml, preferably from 100 ng/ml to 2.5 mg/ml, more preferably from 1 μg/ml to 500 μg /ml, even more preferably from 5 μg/ml to 100 μg/ml and still more preferably from 10 to 50 μg/ml. In a particularly preferred case the RNA concentration may be from 15 to 40 μg/ml, preferably from 20 to 35 μg/ml and in particular may be 25 μg/ml. In some cases, a total of 100 ng to 0.1 g, preferably from 1 μg to 50 mg, more preferably from 100 g to 10 mg, still more preferably from 250 μg to 1 mg of RNA may be administered. Any suitable concentration and/or amount of RNA may be provided. A wide range of concentrations and/or amounts of RNA may be employed and the precise concentration and/or amount may be varied according to the method of delivery of the RNA to the subject and the source of the RNA. The concentration and/or amount of RNA employed may also be adjusted depending on the mass of the subject to be treated. Once aware of the teaching of the present invention, it would be routine to the skilled reader to optimise the amount of RNA provided to the subject in order to bring about the desired treatment response.

Accordingly, this aspect of the invention also provides an in vivo method of directing differentiation of cell fate towards a function or property of one or more desired cell types or tissues comprising providing isolated RNA comprising an RNA sequence extractable from cells comprising said desired cell type(s) to a population of cells under conditions whereby the desired differentiation of said cells is achieved. The RNA may be extractable or extracted from cells comprising said desired cell type(s). The population of cells is preferably a tissue, such as an isolated tissue grown outside the body, or an organism such as a human patient.

The invention also provides a method of improving or rectifying tissue or cellular damage or a genetic disease in a subject, the method comprising inducing totipotent, pluripotent or unipotent resident stem cells in the subject to differentiate (e.g. with mobilisation, migration, integration and proliferation) into one or more desired cell types (e.g. at the target location), which method comprises providing isolated RNA sequence comprising RNA extractable from tissue or cells comprising said desired cell type(s) to the resident stem cells in situ, whereby the desired differentiation (e.g. with mobilisation, migration, integration and proliferation) of said stem cells is achieved. The RNA may be extractable or extracted from cells comprising said desired cell type(s). The method of the invention may be used to improve stem cell-mediated repair, either in vivo or in vitro.

In one embodiment, this aspect of the present invention provides a method of inducing totipotent, pluripotent or unipotent stem cells in tissue of an animal or plant to differentiate into one or more desired cell types, which comprises providing isolated RNA comprising RNA sequence extractable from tissue or cells comprising said desired cell type(s) to said stem cells under conditions whereby the desired differentiation of said stem cells is achieved. The RNA may be extractable or extracted from cells comprising said desired cell type(s). The stem cells reside and are exposed to the RNA in situ, in the organism.

In other embodiments of the invention, the method may be used to confer desired properties of one cell type onto another, optionally in situ in a subject. For example, desired properties possessed by a particular cell type may be conferred on target cells by extracting RNA from the cell type with the desired properties and exposing target cells to this RNA. Examples include extraction of RNA from muscle cells of trained athletes, so as to confer a desired function in a treated subject; transferral of resistance to disease from a vaccinated or naturally resistant individual; and boosting the immune function of a diseased subject.

In some cases the medicaments and methods of the invention may involve the RNAs of the invention being provided to the target stem cells in situ. This may result in resident stem cells differentiating to give rise to the desired differentiated cell type. Such an approach may be used for any of the above-mentioned conditions and disorders. In such an approach the RNA will typically be delivered so that it only affects a relatively localised population of stem cells. Preferably, the stem cells targeted may be those that give rise to the particular cell type involved in the disorder, but this may not always be the case. For example, the subject may have an immune system disorder and haematopoietic stem cells may be targeted.

Delivery to the chosen population of stem cells may be achieved by providing the RNA locally, such as to the appropriate tissue or organ. For example, the administration of the RNA may be intravenous, rectal, oral, auricular, intraosseous, intra-arterial, intramuscular, subcutaneous, cutaneous, intradermal, intracranial, intratheccal, intraperitoneal, topical, intrapleural, intra-orbital, intra-cerebrospinal fluid, intranodal, intralesional, transdermal, intranasal (or other mucosal), pulmonary, or by inhalation to a site of interest. The RNA may, for example, be provided by local injection. The RNA may be provided by injection into a blood vessel or other vessel that leads to the desired target site. The RNA may be administered by local injection to the desired tissue. The RNA may be administered by any of the routes mentioned herein such as intra-muscular injection or by ballistic delivery. In some cases the localised delivery may be achieved because the RNA is provided in a form that specifically targets the RNA to the chosen cells. For example, the RNA may be provided in liposomes or other particles that have targeting molecules for the specific desired stem cell type. In preferred embodiments the RNA may be administered via direct organ injection, vascular access, or via intramuscular, intra-peritoneal, or sub-cutaneous routes. In one preferred embodiment administration of an RNA is achieved as follows:
an RNA extract is prepared from desired tissue type including any of those mentioned herein;
the RNA is injected either directly to affected organ or via systemic delivery as defined above; and
the RNA induces resident stem cell differentiation resulting in, for example, proliferation of the desired cell type, migration and repair.

In some embodiments, the RNA sequence is extractable from or extracted from one or more differentiated cell types. For example, in a specific embodiment, the RNA is derived from primary tissue, such as brain tissue. In other embodiments, the RNA is extractable from one or more stem cell types or stem cell active tissue(s). For example, in a specific embodiment, the RNA is derived from adult stem cells, such as bone marrow stem cells. In another specific embodiment, the RNA is derived from foetal, neonatal, juvenile or embryonic tissue(s).

In a specific example of this aspect of the invention, the invention provides a method for treating human patients with Motor Neuron Disease. In order to treat this disease, it is necessary to effect the regeneration of alpha motor neurons and associated cells in the spine. Accordingly, within the context of this example, these cell types may constitute the target tissue. An example of a suitable source tissue for obtaining RNA for use in this example would be a sample of spinal cord from a human donor cadaver. Suitable RNA samples for use in this example include RNA obtained by extraction from this source tissue, as described below. The RNA is administered to the patient as described below, for example by intravenous injection. Regeneration of alpha-motor neurones and lessening of symptoms in the patient is achieved.

b) Therapy Using Stem Cells Treated with an RNA of the Invention

In a further aspect of the invention, stem cells treated with an RNA of the invention may be administered to the subject.

Accordingly, this aspect of the invention provides a method of effecting a treatment response in a target tissue of a subject, comprising administering to the subject stem cells such that said treatment response is effected, wherein said stem cells have been treated with isolated RNA comprising an RNA sequence extractable from a source tissue. The RNA may be extractable or extracted from the source tissue.

This aspect of the invention also provides for the use of stem cells that have been treated with isolated RNA comprising an RNA sequence extractable from a source tissue in the manufacture of a medicament for effecting a treatment response in a target tissue of a subject. The RNA may be extractable or extracted from the source tissue.

The RNA may be provided to the stem cells by any suitable technique. A number of methods for the provision of nucleic acid molecules to cells are known and these may be employed. For example, suitable techniques may include calcium phosphate transfection, DEAE-Dextran, electroporation, liposome encapsulation, liposome-mediated transfection, microsphere encapsulation, transduction using viral envelope particles and microinjection. The calcium phosphate precipitation method of Graham & van der Eb (1978) may be employed. General aspects of mammalian cell host system transformations have been described in U.S. Pat. No. 4,399,216 and may be employed. For various techniques for transforming mammalian cells, see Keown et al. (1990) and Mansour et al. (1988). In some cases the RNA or the enclosed RNA may be bound to chemical agents that enhance uptake by the target cells. For example the RNA of RNA-containing particles may be linked to an antibody specific to an appropriate receptor. Such a targeting chemical may increase uptake by all cell types, or may have an effect which is specific to a particular stem cell type. As an alternative, RNA can be administered without being bound to such reagents e.g. naked RNA. In some cases the RNA may simply be added to the culture medium of the cells. Suitable culture conditions are well known in the art (Freshney (2000) Culture of Animal Cells, Wiley-Liss NY). In particular, the culture may be incubated in an atmosphere of 5% CO₂/air at 37° C. The RNA may be added to the culture medium of cells for a suitable period of time. For example, the cells and RNA may be cultured together for from 1 minute to 10 days, preferably from 1 hour to 5 days, more preferably from 6 hours to 2 days. In a preferred embodiment the RNA may be cultured with the cells for 12 or 24 hours and in particular for 12 hours. In another example, the cells and RNA may be cultured together for prolonged periods from 10 days to 60 days. In a preferred embodiment of this example, the RNA may be cultured with the cells for 10 to 14 days. In a further example, the RNA may be repeatedly administered to the cells hourly, daily or weekly. Similar time periods may be employed where the RNA is provided in the form of liposomes comprising the RNA sequences or by any of the other methods for providing RNA outlined above.

In some embodiments, the temperature of the cells in culture may be lowered or raised to facilitate uptake of the RNA. The cells are typically maintained at a constant pH. In some embodiments, the cells may be osmotically shocked to facilitate RNA uptake. The culture conditions may contain specified serum, or may be serum free. In some embodiments, the media may be conditioned by specific tissues or cell types. In other examples, the cells may be grown on a defined substrate (e.g. gelatin, polylysine, feeder cell layer etc.)

The amount of RNA provided to the stem cells will be sufficient to bring about the desired alteration in a cell property. For example, the concentration of RNA may be from 10 ng to 5 mg/ml, preferably from 100 ng/ml to 2.5 mg/ml, more preferably from 1 μg/ml to 500 μg/ml, even more preferably from 5 μg/ml to 100 μg/ml and still more preferably from 10 to 50 μg/ml. In a particularly preferred case the RNA concentration may be from 15 to 40 μg/ml, preferably from 20 to 35 μg/ml and in particular may be 25 μg/ml. In some cases, a total of 100 ng to 0.1 g, preferably from 1 μg to 50 mg, more preferably from 100 μg to 10 mg, still more preferably from 250 μg to 1 mg of RNA may be provided. Any suitable concentration and/or amount of RNA may be provided. A wide range of concentrations and/or amounts of RNA may be employed and the precise concentration and/or amount may be varied according to the method of delivery of the RNA to the cells and the source of the RNA. Once aware of the teaching of the present invention, it would be routine to the skilled reader to optimise the amount of RNA provided to the cells in order to bring about the desired alteration in a cell property.

The stem cells used are totipotent, pluripotent or unipotent stem cells, preferably derived from a tissue of the subject to be treated, or of a stem cell line, again preferably derived from a tissue of the subject to be treated. In other words, the stem cells are preferably autologous stem cells. The subject may be suffering from any one or more of the disorders and conditions mentioned herein.

The amount of RNA-treated stem cells administered to the subject will be sufficient to bring about the desired treatment response. For example the concentration of cells (e.g. in a medicament of the invention) may be from 1×10³ to 20×10⁶/ml, preferably from 1×10⁶ to 20×10⁶/ml, more preferably from 1×10⁶ to 5×10⁶/ml, even more preferably from 2×10⁶ to 4×10⁶/ml and still more preferably from 3×10⁶ to 4×10⁶/ml. In some cases, a total of 10² to 10⁹ cells may be administered. Any suitable concentration and/or amount of cells may be administered. A wide range of concentrations and/or amounts of cells may be employed and the precise concentration and/or amount may be varied according to the method of delivery of the cells to the subject and the source of the cells. The concentration and/or amount of cells employed may also be adjusted depending on the mass of the subject to be treated. Once aware of the teaching of the present invention, it would be routine to the skilled reader to optimise the amount of cells provided to the subject in order to bring about the desired treatment response. For example, in one embodiment, differentiated cells obtained using the invention may be administered to the subject. Any of the differentiated cell types mentioned herein may be administered and the subject may be suffering from any one or more of the disorders and conditions mentioned herein.

In a some embodiments, the cells may be administered relatively soon (e.g. within 24 hours, or more preferably within 18, 12, 8, 6, 4, 2 hours or most preferably within 1 hour) after treatment with RNA. In other embodiments, the cells may be administered a relatively long time (e.g. within 10 days, more preferably within 9, 8, 7, 6, 5, 4, 3, 2 days or most preferably within 1 day) after treatment with RNA.

In a specific embodiment of this aspect of the invention, the invention provides a method of improving or rectifying tissue or cellular damage or a genetic disease in a subject, the method comprising administering to the subject an effective amount of differentiated cells obtained in vitro by inducing totipotent, pluripotent or unipotent stem cells, preferably derived from a tissue of the subject to be treated, or of a stem cell line, again preferably derived from a tissue of the subject to be treated, to differentiate into one or more desired cell type(s), which comprises providing isolated RNA comprising RNA extractable from tissue or cells comprising said desired cell type(s) to a cell culture of said stem cells under conditions whereby the desired differentiation of said stem cells is achieved.

In another specific embodiment, the treatment of stem cells with an RNA according to the invention prior to administration of the stem cells to a subject has the effect of enhancing the mobilisation, migration, integration, proliferation and/or differentiation of the stem cells in the subject. In preferred embodiments, the stem cells are treated with RNA sequence that is extractable from or extracted from one or more differentiated cell types, in accordance with any one of the embodiments of the invention described herein. For example, in one specific embodiment, bone marrow stem cells may be pre-treated with brain RNA prior to their administration to a subject, such as a subject suffering from age-related damage to the brain. This has been demonstrated herein successfully to reverse and thus treat age-related disease of the brain in a rat model. In another specific embodiment, bone marrow stem cells may be pre-treated with spine RNA, prior to their administration to a subject, such as a subject suffering from motor neurone disease. This has been demonstrated herein to be effective in an acknowledged model of motor neurone disease.

In a further specific embodiment stem cells with altered properties, obtained using the invention, may be administered to the subject. Accordingly, the invention provides a method of improving or rectifying tissue or cellular damage or a genetic disease in a subject, the method comprising administering to the subject an effective amount of cells with altered properties, evident or latent, obtained in vitro by altering the properties of totipotent, pluripotent or unipotent stem cells of a stem cell line derived from the subject to be treated or stem cells obtained from a tissue of the subject to be treated. Said method comprises providing isolated RNA comprising RNA extractable from tissue or cells comprising the desired cell type(s) or tissue to a cell culture of said stem cells under conditions whereby the desired alteration of properties of said stem cells is achieved. In one preferred embodiment, the cells may be administered relatively soon (e.g. within 24 hours, or more preferably within 18, 12, 8, 6, 4, 2 hours or most preferably within 1 hour) after treatment in vitro with RNA, at a time when several of the altered properties are latent rather than evident, and where the later stages of migration, integration, proliferation, and differentiation may occur in vivo in the recipient. In another embodiment, the cells may be administered only when proliferation and differentiation have been evidenced. In such embodiment, the cells may be administered, for example, within 1 day to 60 days (or possibly longer) after treatment in vitro with RNA.

In specific examples of this embodiment, cells with altered properties, latent or evident, related to any of the differentiated cell types mentioned herein may be administered and the subject may be suffering from any of the disorders and conditions mentioned herein.

In all of these embodiments, the RNA-treated stem cells may be administered to the localised site affected by the disorder. For example, they may be delivered to the pancreas in the case of diabetes, to the spinal nerve in the case of spinal injury, to the brain for brain disorders and so on. Other examples will be clear to the reader of skill in the art, in view of the teaching presented herein. In some cases the said cells may be provided to the subject present on, or as part of, a structure. For example, stents coated with said cells may be inserted into a blood vessel or liver cells may be provided on a matrix to a damaged or diseased liver. In another embodiment the RNA-treated stem cells may be administered on a more general basis, for example by introduction into the circulation, peritoneum, into the cerebrospinal fluid, intrapleurally and so on.

Delivery of the RNA-treated stem cells may be achieved by providing the cells locally, such as to the appropriate tissue or organ. For example, the administration of the cells may be intravenous, intraosseous, intra-arterial, intramuscular, subcutaneous, cutaneous, intradermal, intracranial, intratheccal, intraperitoneal, topical, intrapleural, intra-orbital, intra-cerebrospinal fluid, intranodal, intralesional, transdermal, intranasal (or other mucosal), pulmonary, inhalation, to a site of interest. The cells may, for example, be provided by local injection. The cells may be provided by injection into a blood vessel or other vessel that leads to the desired target site. The cells may be administered by local injection to the desired tissue. The cells may be administered by any of the routes mentioned herein such as intramuscular injection. In preferred embodiments the cells may be administered via direct organ injection, vascular access, or via intramuscular, intra-peritoneal, or sub-cutaneous routes.

In a specific example of this aspect of the invention, the invention provides a method for treating human patients with Motor Neuron Disease. In order to treat this disease, it is necessary to effect the regeneration of alpha motor neurons and associated cells in the spine. Accordingly, within the context of this example, these cell types constitute the target tissue. An example of a suitable source tissue for obtaining RNA for use in this example would be a sample of spinal cord from a human donor cadaver. Suitable RNA samples for use in this example include RNA obtained by extraction from this source tissue, as described below. Examples of suitable stem cells for use in this example would include a culture of bone marrow-derived mesenchymal stem cells, preferably derived from the patient to be treated. Such cells may be prepared, for example, by obtaining a bone marrow sample from the donor and culturing stem cells derived from this sample ex vivo. These stem cells are then treated in vitro with the RNA in order to alter their properties, as described infra. The RNA-treated stem cells are administered to the patient as described below, for example by intravenous injection. Regeneration of alpha-motor neurones and lessening of symptoms in the patient is achieved.

### c) RNA and Stem Cell Combination Therapy

RNA according to the invention may be applied to the subject in conjunction with other active agents, including, for example, stem cells (which may or may not have been treated with an RNA of the invention). The RNA and other active agents may be administered simultaneously, sequentially or separately.

Accordingly, this aspect of the invention provides a method of effecting a treatment response in a target tissue of a subject, comprising

i) administering to the subject stem cells, wherein said stem cells may or may not have been treated with isolated RNA comprising an RNA sequence extractable from a source tissue; and

ii) administering to the subject isolated RNA comprising an RNA sequence extractable from a source tissue. The RNA may be extractable or extracted from the source tissue. Steps i) and ii) may be performed in any order. Moreover, steps i) and ii) may be performed simultaneously, sequentially or separately.

This aspect of the invention also provides for the use of stem cells that may or may not have been treated with isolated RNA comprising an RNA sequence extractable from a source tissue in the manufacture of a medicament for effecting a treatment response in a target tissue of a subject, wherein said medicament is for simultaneous, separate or sequential administration with isolated RNA comprising an RNA sequence extractable from a source tissue. The RNA may be extractable or extracted from the source tissue.

Similarly, this aspect of the invention also provides for the use of isolated RNA comprising an RNA sequence extractable from a source tissue in the manufacture of a medicament for effecting a treatment response in a target tissue of a subject, wherein said medicament is for simultaneous, separate or sequential administration with stem cells that may or may not have been treated with isolated RNA comprising an RNA sequence extractable from a source tissue. The RNA may be extractable or extracted from the source tissue.

In one embodiment of this aspect, medicaments comprising stem cells may be administered to the subject and then a medicament comprising an RNA capable of inducing differentiation in accordance with the invention may be administered in order to induce differentiation or alteration of the stem cells in situ. In another embodiment, the stem cells may be introduced subsequent to the introduction of the RNA.

As in integer b) above, the stem cells used may be totipotent, pluripotent or unipotent stem cells, preferably derived from a tissue of the subject to be treated, or of a stem cell line, again preferably derived from a tissue of the subject to be treated. In other words, the stem cells are preferably autologous stem cells. The subject may be suffering from any of the disorders and conditions mentioned herein.

As in integer b) above, the amount of (optionally RNA-treated) stem cells administered to the subject will be sufficient to bring about the desired treatment response. For example the concentration of cells (e.g. in a medicament of the invention) may be from 1×10³ to 20×10⁶/ml, preferably from 1×10⁶ to 20×10⁶/ml, more preferably from 1×10⁶ to 5×10⁶/ml, even more preferably from 2×10⁶ to 4×10⁶/ml and still more preferably from 3×10⁶ to 4×10⁶/ml. In some cases, a total of 10² to 10⁹ cells may be administered. Any suitable concentration and/or amount of cells may be provided. A wide range of concentrations and/or amounts of cells may be employed and the precise concentration and/or amount may be varied according to the method of delivery of the cell to the subject and the source of the cells. The concentration and/or amount of cells employed may also be adjusted depending on the mass of the subject to be treated. Once aware of the teaching of the present invention, it would be routine to the skilled reader to optimise the amount of cells provided to the subject in order to bring about the desired treatment response.

Preferably, the stem cells in this aspect of the invention have been treated with isolated RNA comprising an RNA sequence extractable from a source tissue, as described in integer b) above. In another preferred embodiment, the stem cells in this aspect of the invention have not been treated with isolated RNA comprising an RNA sequence extractable from a source tissue.

Again as in integer b) above, the stem cells may be administered to the localised site affected by the disorder. For example, cells may be delivered to the pancreas in the case of diabetes, to the spinal nerve in the case of spinal injury, to the brain for brain disorders and so on. In some cases the said cells may be provided to the subject present on, or as part of, a structure. For example, stents coated with said cells may be inserted into a blood vessel or liver cells may be provided on a matrix to a damaged or diseased liver. In another embodiment the cells may be administered on a more general basis, for example by introduction into the circulation, peritoneum, into the cerebrospinal fluid, intrapleurally and so on.

Again, as in integer b) above, delivery of the stem cells may be achieved by providing the cells locally, such as to the appropriate tissue or organ. For example, the administration of the cells may be intravenous, intraosseous, intra-arterial, intramuscular, subcutaneous, cutaneous, intradermal, intracranial, intratheccal, intraperitoneal, topical, intrapleural, intra-orbital, intra-cerebrospinal fluid, intranodal, intralesional, transdermal, intranasal (or other mucosal), pulmonary, by inhalation, to a site of interest. The cells may, for example, be provided by local injection. The cells may be provided by injection into a blood vessel or other vessel that leads to the desired target site. The cells may be administered by local injection to the desired tissue. The cells may be administered by any of the routes mentioned herein such as intramuscular injection. In preferred embodiments the cells may be administered via direct organ injection, vascular access, or via intra-muscular, intra-peritoneal, or sub-cutaneous routes.

In a specific example of this aspect of the invention, the invention provides a method for treating human patients with Motor Neuron Disease. In order to treat this disease, it is necessary to effect the regeneration of alpha motor neurons and associated cells in the spine. Accordingly, within the context of this example, these cell types constitute the target tissue. An example of a suitable source tissue for obtaining RNA for use in this example includes a sample of spinal cord from a human donor cadaver. Examples of suitable RNA samples for use in this example include RNA obtained by extraction from this source tissue, as described below. An example of suitable stem cells for use in this example include a culture of bone marrow-derived mesenchymal stem cells, preferably derived from the patient to be treated. Such cells may be prepared, for example, by obtaining a bone marrow sample from the patient and culturing stem cells derived from this sample ex vivo. In one example, these stem cells are treated in vitro with the RNA in order to alter their properties, as described infra. In another example, the stem cells are not treated in vitro with the RNA in order to alter their properties, as described infra. The (optionally RNA-treated stem cells) are administered to the patient as described below, for example by intravenous injection. The RNA may also be administered to the patient as described below, for example by intravenous injection. A degree of regeneration of alpha-motor neurones and lessening of symptoms in the patient is achieved.

### d) Combinatorial Therapy

Combinations of integers a), b) and c) may also be employed. For example, RNA-treated stem cells and RNA of the invention may be administered in simultaneous, separate or sequential application (e.g. in a combination of a) and b) above). Combinations of integers b) and c) are also specifically envisaged. For example, RNA-treated stem cells and stem cells that have not been treated with RNA of the invention may be administered in simultaneous, separate or sequential application with RNA of the invention.

Cells and RNA of the invention may also be administered in simultaneous, separate or sequential application with other therapies effective in treating a particular disease. In one embodiment, RNA extractable from one or more stem cell types or stem cell active tissue(s) may be administered in simultaneous, separate or sequential application with cells, such as stem cells. For example, in preferred embodiments, whole embryo RNA, foetal RNA, neonatal or juvenile RNA may be administered in simultaneous, separate or sequential application with stem cells, particularly bone marrow stem cells. It is shown here that stem cell mediated tissue repair and regeneration is improved by co-injecting embryo-derived RNA fractions with stem cells.

In any of the combinatorial therapies of the present invention, the individual treatments (i.e. a), b) or c)) may be applied in a discrete, sequential application. This means that the individual treatments are applied sequentially, with each treatment being completed before commencement of the subsequent treatment. In such embodiments, there may be a defined sequence to the treatments and/or there may be a defined length of time between each treatment.

Alternatively, combinatorial therapies of the present invention may involve the individual treatments being applied in an overlapping application. This means that the individual treatments are applied sequentially, with each treatment overlapping at least partially with one or more of the other treatments. In such embodiments, there may be a defined sequence to the treatments and/or there may be a defined length of time to the overlap between each treatment. In an extreme version of this embodiment, one or more of the treatments are applied simultaneously.

Although the invention has very broad applications, the invention may in particular be used to treat or ameliorate degenerative brain disease, brain or spinal cord injury or other neuronal disorders. In preferred embodiments the cells may be provided to a subject suffering from a degenerative disease and in particular an age related degenerative disease. The disease or damage to be treated with the medicaments of the invention may affect the brain. The subject may, for example, be suffering from a degenerative brain disease. Examples of brain disorders include, in particular, Parkinson's disease, Parkinsonian type disorders, Alzheimer's, dementia, other age related brain pathologies and Motor neurone disease. Multiple sclerosis may also be treated. Another disorder that may be treated is diabetes and particularly type 1 and type 2 diabetes, by providing insulin producing islet of Langerhans cells to replace or augment the defective cells. The invention may also be used for subjects suffering from disorders caused by damage to joints such as, for example, arthritis.

The invention also provides an agent for improving or rectifying tissue or cellular damage or a genetic disease, the agent comprising the RNA or differentiated cells (or cells with altered properties, latent or evident) as defined herein, or a combination of both. For example the invention provides for the treatment of degenerative diseases and age-related degeneration of any organ for example, heart disease, congestive heart failure, cardiac valve dysfunction, venous valve dysfunction, degenerative kidney disease, and degenerative liver disease. The invention also provides for regeneration of tissue after damage due vascular accident for example, ischemia, thrombosis, aneurism, and pressure sores.

The invention also provides a method for regeneration, repair or replacement of tissue(s) damaged or lost through pathology, age, or trauma of any description. For example the invention provides a method for regeneration and repair following traumatic damage to the spinal column.

In the above methods of treating a subject the stem cell, differentiated cells, altered cells, RNA, method of providing the RNA and other aspects may be as defined anywhere herein. In respect of the above agents, the RNA or differentiated cell or altered cell may be any defined herein.

### Stem Cells

The invention may use any suitable stem cell, preferably derived from the subject to be treated. In other words, the stem cells used in the methods and medicaments of the present invention are preferably autologous stem cells.

A stem cell is generally understood to be a cell capable of self-renewal that is also capable of differentiation into one or more specific differentiated cell type(s). Stem cells may be pluripotent, that is, they may be capable of giving rise to a plurality of different differentiated cell types. In some cases the stem cells may be totipotent, that is they may be capable of giving rise to all of the different cell types of the organism that they are derived from. In other cases the stem cells may be unipotent (i.e. they may be “progenitor stem cells”), that is they may be capable of giving rise to one cell type of the organism from which they are derived. The invention is applicable to pluripotent stem cells, totipotent stem cells or unipotent stem cells, including the types of stem cells as described below.

Preferably, the invention uses stem cells from an adult, juvenile, baby, fetus or embryo. In some embodiments, for example when the use of stem cells derived from fetuses or embryos is prohibited by law, the stem cells are from an adult, juvenile or baby.

Stem cells are known to occur in a number of locations in the human body. Stem cells used in the present invention may be those from any of the organs and tissues in which stem cells are present. Examples include stem cells from the bone marrow, haematopoietic system, neuronal system, the brain, muscle stem cells or umbilical cord stem cells. The stem cells may in particular be bone marrow mesenchymal stem cells, foetal mesenchymal stem cells, blood-derived stem cells (e.g. CD34 positive circulatory stem cells), Tristem™ stem cells, spinal stem cells, neural stem cells, umbilical-cord derived stem cells, skin-derived stem cells, gut-derived stem cells, fat-derived stem cells and muscle-derived stem cells. Other examples include foetal-derived stem cells, placental-derived stem cells and tissue type-specific progenitor stem cells, such as retinal stem cells, liver stem cells, satellite cells, neuronal progenitors, glial progenitors, fibroblasts, olfactory ensheathing cells and reproductive system stem cells.

In a particularly preferred embodiment the invention is used to differentiate adult stem cells. Stem cells are known to occur in a number of locations in the human body. Stem cells differentiated by the present invention may be those from any of the organs and tissues in which stem cells are present. Examples include stem cells from the bone marrow, haematopoietic system, neuronal system, the brain, muscle stem cells or umbilical cord stem cells. The stem cells may in particular be bone marrow stromal stem cells, neuronal stem cells or haematopoietic stem cells, in a preferred case they may be bone marrow stromal stem cells or neuronal stem cells. In particular, when the methods of the invention include the ex vivo treatment of stem cells with an RNA of the invention, the stem cell may be a bone marrow stromal cell.

In many cases, the differentiated cells may be intended to treat a subject, and in the manufacture of medicaments. In such cases the stem cells may preferably be from the intended recipient. Accordingly, the stem cells are usually autologous. In other cases the stem cells may originate from a different subject. Accordingly, the stem cells may be allogeneic. However when the stem cells are allogeneic, they are preferably chosen to be immunologically compatible with the intended recipient. For example, the donor may be chosen to have an immunological profile which has a specific relationship to the immunological profile of the recipient. Accordingly, in some cases the stem cells may be from a relation of the intended recipient such as a sibling, half-sibling, cousin, parent or child, and in particular from a sibling. The stem cells may be from an unrelated subject who has been tissue-typed and found to have a immunological profile which will result in no immune response or only a low immune response from the intended recipient which is not detrimental to that recipient. However, in other cases the stem cells may be from an unrelated subject. For example, the stem cell and the recipient may or may not have a histocompatible haplotype (e.g. HLA haplotypes).

In some cases the stem cells may be embryonic stem cells, foetal stem cells, neonatal stem cells, or juvenile stem cells. The embryonic, foetal, neonatal, or juvenile stem cells may be pluripotent stems cells and particularly totipotent stem cells. The cells may be from any stage or sub-stage of development, in particular they may be derived from the inner cell mass of a blastocyst (e.g. embryonic stem cells). In some cases, the embryonic, foetal, neonatal or juvenile stem cells may be recovered and then used in the manufacture of medicaments to treat the subject at some stage in their life. In one embodiment, where embryonic, foetal, neonatal or juvenile stem cells are employed, they will be from already established foetal, embryonic, neonatal or juvenile stem cell lines. This will particularly be the case for human cells. In some cases the stem cells may be obtained from, or derived from, extra-embryonic tissues. The stem cells may be obtained from the umbilical cord and in particular from umbilical cord blood.

In certain jurisdictions, for reasons of public policy, the stem cells may not be totipotent stem cells that have the capacity to form a human being. This is particularly the case where the stem cells are human foetal or embryonic stem cells.

The invention is also applicable to stem cell lines. Stem cell lines are generally stem cell populations that have been isolated from an organism and maintained in culture. Thus the invention may be applied to stem cell lines including adult, foetal, embryonic, neonatal or juvenile stem cell lines, preferably derived from the subject to be treated. The stem cell lines may be a clonal stem cell line i.e. they may have originated from a single stem cell. In one preferred embodiment the invention may be applied to existing stem cell lines, particularly to existing embryonic and foetal stem cell lines. In other cases the invention may be applied to a newly established stem cell line. This will particularly be the case when the stem cell line is derived from the subject to be treated.

The stem cells may be an existing stem cell line. Examples of existing stem cell lines which may be used in the invention include the human embryonic stem cell line provided by Geron and the neural stem cell line provided by Reneuron. In a preferred case the stem cell line may be one which is a freely available stem cell, access to which is open, and in particular such an existing stem cell line.

In the case of human embryonic stein cell lines, in a preferred case a pre-existing stem cell line will be used. In a particularly preferred embodiment of the invention, where a human embryonic stem cell line is used, the cell line may be one where the derivation process (which begins with the destruction of the embryo) was initiated prior to 9:00 p.m. EDT on Aug. 9, 2001. Preferably human embryonic stem cell lines may be ones created from embryos donated for reproductive purposes which were no longer needed for the original purpose, because, for example, they were surplus to requirements. Preferably informed consent will have been obtained for the use of the embryos to create the cell line. In a preferred case, the human embryonic stem cell line employed will meet the requirements announced by President Bush on 9 Aug. 2001 as being necessary for obtaining US federal funding for embryonic stem cell research. These include the stem cell lines recognised as meeting the requirements from BresaGen Inc. of Australia; CyThera Inc.; the Karolinska Institute of Stockholm, Sweden; Monash University of Melbourne, Australia; National Centre for Biological Sciences of Bangalore, India; Reliance Life Sciences of Mumbai, India; Technion-Israel Institute of Technology of Haifa, Israel; the University of California at San Francisco; Goteborg University of Goteborg, Sweden; and the Wisconsin Alumni Research Foundation.

Reference herein to stem cell generally includes the embodiment mentioned also being applicable to stem cell lines unless, for example, it is evident that the target cells are freshly isolated stem cells or the stem cells are resident stem cells in vivo. The invention is applicable to freshly isolated stem cells and also to cell populations comprising stem cells. The invention may also be used to control the differentiation of stem cells in vivo.

An initial step in the methods of the invention may be the isolation of suitable stem cells. Methods for isolating particular types of stein cells are well known in the art and may be used to obtain stem cells for use in the invention. The methods may, for example, be used to recover stem cells from the intended recipients of the medicaments of the invention. Cell surface markers characteristic of stem cells may be used to isolate the stem cells, for example, by cell sorting. In particular embodiments, stem cells are obtained from tissue samples by a know method of culturing, for example the method of obtaining bone marrow mesenchymal stem cells from bone marrow or the method of obtaining cultures of CD34 positive stem cells from blood, bone marrow, spleen or liver. Stem cells may be obtained from subjects suffering from any of the disorders mentioned herein.

The stem cells may be freshly isolated stem cells or they may be an ex-vivo culture of stem cells. They may also be obtained from one or more primary cell lines derived from any of the above examples. In some cases, the stem cells may be isolated from a subject, differentiated in vitro and then returned to the same subject. Such ex vivo methods are particularly preferred.

In some cases the target stem cells may exist in situ, that is, they may be present in the subject to be treated. Thus, in a further aspect the invention provides for the use of an RNA in accordance with the invention which is capable of inducing differentiation of stem cells in the manufacture of a medicament for use in improving or rectifying tissue or cellular damage or a genetic disease. The invention also embraces methods that use isolated RNA in accordance with the invention, which is capable of inducing differentiation of stem cells in improving or rectifying tissue or cellular damage or treating a genetic disease. Such a method may, for example, be used for treating a degenerative brain disease or brain or spinal cord injury. It may also be used for the treatment of diseases such as liver disease, heart disease, skeletal or cardiac muscle disease and type I diabetes. Furthermore, it may be used to counteract age-related degenerative disease. Other examples will be clear to those of skill in the art.

In such embodiments, the stem cells may be any of the types of stem cells mentioned herein. The target stem cells may be present in any of the organs, tissues or cell populations of the body in which stem cells exist, including any of those mentioned herein. The target stem cells will typically be resident stem cells naturally occurring in the subject, but in some cases stem cells that have been transferred into the subject may be the target stem cells.

Various techniques for isolating, maintaining, expanding, characterising and manipulating stem cells in culture are known and may be employed. In some cases genetic modifications may be introduced into the genomes of the stem cells. Stem cells lend themselves to such manipulation as clonal lines can be established and readily screened using techniques such as PCR or Southern blotting. Techniques such as gene targeting or random integration may be used to introduce changes into the genome of the cells.

In some instances, the stem cells may originate from an individual with a genetic defect. Modifications may then be made to correct or ameliorate the defect. For example, a functional copy of a missing or defective gene may be introduced into the genome of the cell. Gene targeting may be used to introduce desired specific changes and in particular to modify a defective gene to render it normal. Site-specific recombinases may be used to remove selective markers involved in the gene targeting.

The stem cells used in the present invention may consist of a combination of two or more of any of the stem cell types described infra. In some embodiments, this combination may comprise a particular ratio of stem cell types.

The stem cells may be stored before use. Any appropriate method for stem cells storage known in the art may be used.

In some cases the stem cells may be chosen because they have a specific genotype. For example the stem cells may be intended to produce differentiated cells to treat a subject with a genetic defect. The stem cells may lack the genetic defect. For example, the stem cells may be obtained from a relation of the subject who lacks the defect. For example, the cells may be derived from a sibling who does not have the disorder. Alternatively, the stem cells may be stem cells that have been treated ex vivo to correct a genetic defect. This is particularly preferred when the stem cells used in the methods and medicaments of the invention are derived from an intended recipient (i.e. they are autologous stem cells) who has said genetic defect.

The stem cells may be stored before use in the methods and medicaments of the invention. For example, they may have been stored for between 1 hour and 1 year before use in the methods and medicaments of the invention, or alternatively between 6 hours and 6 months, 12 hours and 4 months, 18 hours and 3 months or 24 hours and 1 month. Suitable storage conditions include refrigeration at −80° C. in a suitable freezing solution or at −196° C. (liquid nitrogen) in a suitable freezing solution. An example of a suitable freezing solution would be 90% foetal calf serum, 10% DMSO. Preferred storage conditions are at −196° C. (liquid nitrogen) in a suitable freezing solution.

The stem cells may also have been treated ex vivo to preserve activity before use in the methods and medicaments of the invention. For example, the stem cells may have been treated by being cultured in appropriate media supplemented with LIF and β-mercaptoethanol to maintain them in an undifferentiated state (Bain (1995) Dev Biol 168, 342-357). In another example the cells may have been expanded in culture. In another example the stem cells may have been exposed to specific conditioned media. In another example the stem cells may have undergone a period of co-culture with a second cell type. In another example the stem cells may have been rejuvenated by exposure to appropriate RNA from a cell source of less chronological age or developmental stage.

### RNA Molecules

In order to produce the desired changes in cell properties, the invention employs specific RNA. In general, the RNA employed is one that comprises RNA extractable from tissues or cells comprising the cell type or types that it is desired to induce the target cell to have a cell property in common with. Thus, in embodiments where the aim is to induce differentiation of a stem cell into a desired differentiated cell type, the RNA provided to the target cell is typically an isolated RNA comprising an RNA sequence extractable from tissue or cells comprising the desired differentiated cell type or types. The isolated RNA may comprise an RNA extractable from or extracted from tissue or cells comprising the desired differentiated cell type or types.

Similarly, where the present invention is concerned with the treatment of a subject, for example in those embodiments of the present invention where is it desired to affect a treatment response in a target tissue, the RNA used is one that is extractable from or extracted from a source tissue. As used herein, “source tissue” means one or more tissues or one or more cells types in the donor.

Preferably, the “source tissue” comprises one or more tissues or one or more cell types in common with the target tissue of the subject to be treated. More preferably, the “source tissue” comprises the most abundant tissue or cell type that is present in the target tissue. More preferably still, the “source tissue” comprises the most abundant two or three tissues or cell types that are present in the target tissue. Even more preferably, the “source tissue” comprises all of the tissues or cell types that are present in the target tissue. Most preferably, the “source tissue” consists of all of the tissues or cell types that are present in the target tissue. The target tissue may be entirely homogeneous in nature, in which case the source tissue may consist of that specific tissue.

Preferred examples of target tissue and source tissue and examples of the conditions that they may be used to treat, are given in the following table:

| Target tissue | Source tissue | Condition |
|---|---|---|
| Frontal lobes, parietal lobes, | Hippocampus and/or cortex. | Dementia. |
| occipital, temporal lobes | | |
| and/or hippocampus areas | | |
| known to suffer cell loss in | | |
| dementive illness. | | |
| Motor neurones in the central | Spine. | Motor Neurone Disease. |
| and/or peripheral nervous | | |
| systems. | | |
| Brain and/or spine. | Central nervous system | Multiple Sclerosis. |
| | and/or cultured | |
| | oligodendroglia. | |
| Muscle. | Wildtype dystrophin | Muscular Dystrophy. |
| | producing muscle and/or | |
| | wildtype dystrophin | |
| | producing muscle culture. | |
| Heart wall; valve; or sino- | Whole heart wall, | Various heart disorders. |
| atrial node. | endocardium, myocardium, | |
| | and/or pericardium; valve; or | |
| | sino-atrial node. | |
| Liver. | Hepatocytes (from donor | Liver disease. |
| | organs, biopsy sample and/or | |
| | cultured cells). | |
| Dermal and/or subdermal | Whole skin and/or cultures of | Burns. |
| support tissue. | skin-associated cells. | |
| Skeleton. | Osteoblasts and/or | Orthopaedic damage. |
| | chondrocytes. | |
| Substantia nigra. | Dopaminergic neurones | Parkinson's disease. |
| | (from primary tissue, e.g. | |
| | substantia nigra) and/or | |
| | cultured dopaminergic cells. | |
| Whole organism. | Whole fetus or embryo. | Whole-organism |
| | | deterioration, e.g. due to |
| | | ageing. |

Accordingly, the RNA employed is preferably one that comprises RNA extractable from or extracted from tissue or cells comprising the tissue or cell type or types which it is desired to regenerate or repair. The degree to which the source of the RNA is homogenous will be dictated in part by the specificity of the type of tissue that is desired to be treated. The RNA may be extracted from, or the RNA sequence may be derived from, a particular tissue type, for example, brain (for example, cortex, cerebellum, hippocampus, retina, substantia nigra, subventricular zone), spinal cord, liver, kidney, muscle, nerve tissue (peripheral, central, neuronal, glial), cardiac tissue (for example, atrial, ventricular, valve, cardiac innervation), immune cells, blood, pancreatic tissue, thymic tissue, spleen, skin, and gastrointestinal tract, lung, bone, cartilage, tendon, hair follicle, sense organ (for example, ear, eye), any gland either endocrine, exocrine, or paracrine, such as thyroid, thymus, pituitary, adrenal, pancreatic, reproductive system (for example, testicular, prostate, seminal vesicle, penis, ovarian, uterine, fallopian, mammary), dental, vascular, digestive tract tissues (for example, stomach, gall bladder, intestines, colon). Such tissues are made up of a number of different cell types e.g. constituent cells of brain tissue include various sub-types of neurones and glial cells, vascular tissues, connective tissues and brain-resident stem cells. RNA may be from a specific type of tissue in a particular location, such as a left tibia or left frontal lobe. Accordingly, a more homogeneous population of cells might include neurones and so where the desired treatment is itself specific (for example, in the treatment of age-related brain disease), the RNA may be extracted from neurones, or the RNA sequence may be derived from neurones. More specifically again, the RNA may be from a specific neurone type such as cortical neurones. More specifically again, the RNA may be from a specific type of cortical neurones, such as dopaminergic cortical neurones. In embodiments such as these, the RNA is from a purified cell source.

Source tissue may be derived from one or more donors who are not the subject of the methods or medicaments of the invention. Alternatively, the source tissue may be derived from the subject himself, for which the methods or medicaments of the invention are intended.

In specific embodiments, source tissue may be a whole organism (e. g. a whole embryo, fetus or post-natal cadaver), limb(s), organ(s), part(s) of an organ, an organ from which specific sub-component(s) have been removed, a collection of specific sub-components from organ(s) or specific cell-type(s). Moreover, in any of these embodiments, the source tissue may have had specific cell type(s) (such as cells with one or more particular cell surface makers) completely or partially removed. Similarly, the source tissue may have had specific cell type(s) (such as cells with one or more particular cell surface makers) enriched, or be a selection of such cells.

In other embodiments, source tissue may be an in vitro culture of one or more cell types, or one or more cell lines. Again, in any of these embodiments, the source tissue may have had specific cell type(s) (such as cells with one or more particular cell surface makers) completely or partially removed. Similarly, the source tissue may have had specific cell type(s) (such as cells with one or more particular cell surface makers) enriched, or be a selection of such cells.

In some embodiments, the RNA employed in the invention, derived from a particular tissue type or set of cells or cell lines or cell types, or a cell line or a single cell type, or the RNA sequence derived from such sources, may in addition use a source of such material which comes from a donor of a specific developmental stage. Accordingly, the source tissue may be derived from a donor that is at a different developmental stage to that of the recipient. For instance, the inventors have discovered that the degree of treatment response induced by RNA or RNA-treated stem cells of the invention is influenced by the developmental stage of the source tissue donor. Generally, the younger the age of the donor, the greater the degree of treatment response induced in the recipient by the method. Thus, source tissue may be selected from a donor at a specific developmental stage such as post-implantation embryonic, foetal, neonatal, juvenile, or adult stages. Furthermore, in those embodiments where the source tissue is derived from more than one donor, each donor may be selected such that it is at a specific and optionally different developmental stage.

Accordingly the RNA may be derived from neurones from a particular developmental stage, where that developmental stage is the same as, or earlier than, or later than, the developmental stage of the intended recipient. For example, RNA used in the treatment of cardiac degeneration may be extracted from the cardiac tissue of a juvenile donor. Developmental stages include embryo, foetal, neonatal, juvenile, or adult, or any sub-stage of any of these stages.

In some embodiments the RNA employed in the invention, for the treatment of a tissue or organ in a recipient of a certain developmental stage, may be derived from a tissue or cell type or types that is related to that of the target tissue, but where the exact type of source tissue is only present at a different developmental stage. For example, dental tissue in an adult might be treated with RNA derived from the emergent dental tissue in a neonate or young juvenile.

Other preferred sources of homogenous, purified RNA for use in accordance with the present invention include pure preparations of foetal, neonatal or juvenile cells and pure preparations of embryonic stem cells.

In some preferred embodiments of the invention the RNA employed is derived from stem cells, and is administered into the whole organism, or organ, or tissue. This can cause regeneration and activation of the recipient stem cell population per se, with a secondary consequential regenerative effect on the tissues normally supported by these stem cells. In this case, the RNA provided is typically isolated RNA comprising RNA sequence extractable from a stem cell type or types or stem cell active tissue(s). The RNA may be extractable or extracted from a stem cell type or types or stem cell active tissue(s). Examples of stem cell-rich tissues include foetal tissue and embryo tissue, or tissues from later developmental stages undergoing a phase of growth repair or regeneration.

Typically, a cellular RNA extract will comprise a heterogeneous population of species of different RNA molecules. Types of RNA molecules in a heterogeneous population can include messenger RNA (mRNA), ribosomal RNA (rRNA), transfer RNA (tRNA), heterogeneous nuclear RNA (hnRNA), small nuclear RNA (snRNA), small cytoplasmic RNA (scRNA), small nucleolar RNA (snoRNA), transcription-related RNAs, splicing-related RNAs, signal recognition particle RNAs, linear RNA, circular RNA, inhibitory RNA (e.g. siRNA), single-stranded RNA, double-stranded RNA, etc. It may be desired to treat the cellular RNA extract so as to remove one or more types of RNA molecules that are either unnecessary or even detrimental to the methodology of the invention. In certain embodiments, a population of a small number of or only one RNA species may be prepared.

In a preferred embodiment the RNA will comprise, or consist essentiality of, an RNA extract from source tissue.

Thus, preferably an RNA rich extract is prepared from donor material. The donor material may, for example, be an organotypic source obtained post mortem. However, in other embodiments, the donor is the intended subject of the method or medicament of the present invention. Similarly, in those aspects of the invention wherein stem cells are used, the donor material may be obtained from the same source as the cells to be treated, which is preferably the intended recipient of the cells.

In some embodiments, the source tissue may have been conditioned, modified, treated and/or cultured after extraction from the donor but before extraction of the RNA, such that the nature of the extracted RNA is altered in a desirable manner or the amount of active RNA present prior to extraction is increased. For example, the source tissue may have been treated to remove unwanted RNA sequences partially or completely. This may be achieved, for example, with interference RNA techniques (Liu et al (2004) Eur J Immunology 34,1680-1687). Alternatively, the source tissue may have been treated to increase the amount of active RNA present prior to extraction, for example by treatment with an appropriate medicament. The amount of active RNA present prior to extraction may also be increased by treatment with a RNase inhibitor (e.g. “RNA later” from Ambion) before or after removal from the donor, or after culturing, in order to reduce RNA degradation during the extraction process.

In some embodiments, the source tissue may be preserved at one or more points during the process of RNA extraction, for example by treatment with a RNase inhibitor and/or by storage at −20 to −80° C.

The RNA extract may be from an organ or tissue or cells isolated from an organ or a tissue. For example, the RNA extract may be from an organ, tissue or cells isolated from the group comprising, but not limited to, the brain, spine, heart, kidney, spleen, skin, the gastrointestinal tract or liver. In some embodiments, the source organ, tissue or cells may have been treated one or more times with the methods or medicaments of the present invention. The extract may be from a cell line of specific chosen phenotype, a primary cell culture, or a donor tissue of specific immunological profile.

In some embodiments, the source tissue may be cultured and the culture medium used as the source of, or as an additional source of, RNA of the invention. In such a culture, the cultured tissue may be treated in a particular fashion to modify the amount, activity and/or nature of the RNA released into the medium. For example, the inventors have found that subjecting a cultured tissue to mechanical damage can increase the amount of extractable active RNA. Moreover, in embodiments where a culture medium is used as a source of RNA of the invention, the RNA may be extracted from a fractionated part of that medium, For example, the RNA may be obtained by positive or negative selection of RNA associated with microvesicles, and in particular microvesicles with specific cell surface markers or composed of specific lipids. In another example, the RNA may be derived by positive or negative selection of RNA associated with particular proteins.

Similarly, in some embodiments, the source tissue may have been conditioned and/or modified before extraction from the donor, for example by artificial exercise of function (e. g. physical exercise of a muscle) or by treatment with medicament in order to alter the nature of the extracted RNA in a desirable manner.

Typically, the RNA will comprise RNA sequence that is extractable from the same species as the subject to be treated. However, in some embodiments, the source tissue may be derived from a xenogeneic source. For example, the source tissue may be from a non-human animal and in particular from a non-human mammal. In specific examples of this embodiment, the source tissue may be from a pig, goat, sheep, dog, rodent, fish, amphibian, invertebrate or an animal with innate regenerative capacities, preferably from a pig. In other preferred examples of this embodiment, the source tissue is from a non-human primate such as a monkey. For example, the primate may be a chimpanzee, gorilla or orangutan.

The RNA may comprise an RNA sequence extractable from or an RNA extracted from any of the stem cell types or differentiated cell types mentioned herein.

In some embodiments, the RNA may comprise an RNA sequence extractable from or an RNA extracted from a different developmental stage than the recipient of the cells to be treated. For example, the developmental stage may be more immature than that of the recipient of the cells to be treated. Alternatively, the developmental stage may be a more active cell generative stage. For example, the treatment of spinal cord lesions may be effected by treatment with RNA obtained from donor embryo tissue, sourced at neuralation. The developmental stage may also be one that shows increased stem cell activity. For example, in some preferred embodiments of the invention, the RNA may comprise an RNA sequence extractable from or an RNA extracted from foetal, neonatal juvenile or embryonic developmental stages. For example, where the RNA is extractable from brain cells or tissue, the donor may be at a developmental stage when extensive neurogenesis is occurring, such as the foetal developmental stage. It has been demonstrated by the inventors that provision of RNA extractable from cells of an early developmental stage has advantageous effects, particularly in eliciting stem cell-mediated tissue repair.

The developmental stage may in alternative embodiments be less immature than that of the recipient of the cells to be treated or a less active cell generative stage. In some embodiments, the RNA may comprise an RNA sequence extractable from or an RNA extracted from a tissue that has been pre-treated (for example, chemically or physically) or pre-conditioned (for example, by exercise for muscle tissue or induction of a particular reproductive stage or stage of the female cycle for reproductive tissue) in any way or ways which modify the activity of the extractable RNA. For example, the RNA may be extracted from tissue that has been stressed or damaged.

The specific source tissue used in the present invention will depend on the target tissue in which it is desired to effect a treatment response. The following lists describe specific source tissue for specific target tissue and should be viewed as examples rather than being in any way limiting.

### i) Central Nervous System (CNS)

Tissues of the CNS will constitute the target tissue in various CNS-related conditions. Examples of CNS-related conditions that may be treated by the methods and medicaments of the present invention include motor neurone disease, multiple sclerosis, degenerative diseases of the CNS, dementive illnesses such as Alzheimer's disease, age related dysfunction of the CNS, Parkinson's disease, cerebrovascular accidents, epilepsy, temporary ischaemic accidents, disorders of mood, psychotic illnesses, specific lobe dysfunction, pressure related injury, cognitive dysfunction or impairments, deafness, blindness anosmia, diseases of the special senses, motor deficits, sensory deficits, head injury and trauma to the CNS. Methods and medicaments of the present invention may also be used to enhance brain function or ameliorate deficiencies at a functional level or to facilitate post surgical repair of the CNS.

Source tissue for the treatment of such CNS-related conditions includes whole CNS and subcomponents of the CNS such as whole brain, frontal lobes, parietal lobes, temporal lobes, limbic system, hippocampus, hypothalamus, thalamus, pituitary gland, pineal gland, sub-ventricular zone, olfactory bulb, any such defined anatomical region, nuclei or pathway (e.g. lateral geniculate nuclei, superior or inferior colliculi, dorsal root ganglia and substantia nigra), cerebellum, medulla oblongata, pons, ascending and/or descending tracts of the brain stem, whole or partial fractions of fore, mid, and hind brain structures, grey matter, white matter, specific cranial nerves, corpus callosum, optic chiasm and meningeal structures. In some embodiments, source tissue may comprise individual cell types or cultures enriched for specific cell types. Examples of such cell types include neurones, and specifically motor neurones, sensory neurones, inter-neurones, purkinje cells and pyramidal cells. Other examples include glia, and specifically astroglia, oligodendroglia, microglia, eppendymal cells, cells forming the blood brain barrier and the choroids plexi.

In a specific embodiment, dementia, e.g. Alzheimer's disease, Multi-infarct dementia, pre-senile dementia or other disorders of both short and long term memory, may be treated with the use of tissue from the hippocampus and cortex as source tissue for the RNA. In another specific embodiment, Parkinson's disease may be treated with the use of tissue from the substantia nigra as source tissue. In another specific embodiment, primary and secondary damage caused by cerebrovascular accident may be treated with the use of tissue from the equivalent anatomical region of the brain or brain stem involved as source tissue. In the treatment of autonomic nervous system diseases, tissue from the whole or parts of the autonomic nervous system may be used as source tissue to treat damage in the recipient autonomic nervous system. In demyelinating diseases, myelinated nerves or oligodendroglia may be used as source tissue to remyelinate damaged tissues in the recipient. In another embodiment, ischemic damage to a cortex may be repaired using tissue from the equivalent cortical structures as source tissue. In another specific example, damage to the spinal cord by trauma, disease or congenital defect could be treated using tissue from whole spine or anatomically distinct areas of the spine (appropriate to the specific damage in the recipient) as source tissue. In another specific example, damage to cranial nerves may be treated in a patient with tissue from the appropriate pair(s) of cranial nerves dependent upon the ones damaged in the recipient as source tissue. In another example, diseases and traumatic damage of the basal ganglia including the putamen, pallidus, claustrum, amygdala and caudate nucleus could be treated with tissue from the whole basal ganglia or specifically the putamen, pallidus, claustrum, amygdala and caudate nucleus targeting the repair of specific structures within the defined anatomical locus as source tissue. In another example, where surgical re-attachment of limbs, appendages or parts is required, peripheral nerve tissue may be used as source tissue to facilitate peripheral nerve growth and repair.

### ii) Cardiovascular System

Tissues of the cardiovascular system, and in particular the heart, may constitute the target tissue in various disorders of the cardiovascular system. Examples of disorders of the cardiovascular system that may be treated by the methods and medicaments of the present invention include arrhythmias, myocardial infarction and other heart attacks, pericarditis, congestive heart diseases, valve-related pathologies, myocardial, endocardial and pericardial dysfunctions or degeneration, age-related cardiovascular disorders, dysfunctions, degeneration or diseases, sclerosis and thickening of valve flaps, fibrosis of cardiac muscle, decline in cardiac reserve, congenital defects of the heart or circulatory system, developmental defects of the heart or circulatory system, repair of hypoxic or necrotic damage, blood vessel damage and cardiovascular diseases or dysfunction (e.g. angina, dissected aorta, thrombotic damage, anyurism, atherosclerosis, emboli damage and other problems associated with blood flow, pressure or impediment).

Methods and medicaments of the present invention may also be used to enhance cardiovascular function or health and to revascularise tissues. Moreover, methods and medicaments of the present invention may be used to repair, modify, enhance or regenerate traumatic damage to the heart or blood vessels and as a technique to enhance the transplantation/implantation of a whole organ or its parts. Examples of this latter embodiment include heart transplantation, valve replacement surgeries, implantation of prosthetic devices and the development of novel surgical techniques.

Source tissue for the treatment of such disorders of the cardiovascular system includes whole heart or blood vessels, or subcomponents of the heart or blood vessels such as myocardium, endocardium, pericardium, cardiac muscle bundle, left and/or right atrial tissue, left and/or right ventrical tissue, valve derived tissue, trabeculae carneae, papillary muscles, chordiae tenineae, sinoatrial node, atrioventral node, atrioventricular bundle (bundle of His), aortic tissues, interventricular sulcus (including/excluding interventricular artery), superior or inferior vena cava and sympathetic and/or parasympathetic nervous tissues. Other suitable source tissue includes whole capillary, vein or artery tissue or sub-components of these tissues, including tissues of tunica interna (endothelium and/or subendothelial layer), internal elastic lamina, tunica media, external elastic lamina and tunica externa. In some embodiments, the source tissue may be continuous capillaries, fenestrated capillaries or sinusoids. In some embodiments, source tissue may comprise individual cell types or cultures enriched for specific cell types. Examples of such cell types include cardiac muscle cells, pukinje fibres and endothelial cells.

In a specific embodiment, conduction deficits may be treated with the use of tissue from the SA node or internodal pathways as source tissue. In another specific embodiment, focal calcification type arteriosclerosis may be treated with the use of smooth muscle cells of the tunica media as source tissue. In further specific embodiments, inflammatory heart disease, hypertensive heart disease and congestive heart disease may be treated using as source tissue tissue from the whole heart, heart wall, or pericardium, myocardium, endocardium in total or in parts where damage is to the whole heart, pericardium, myocardium or endocardium. More specifically, tissue from specific anatomically defined regions of the heart, heart wall, valves or blood vessels may be used as source tissue to repair the corresponding structure(s) involved in specific heart pathologies. In another embodiment, rheumatic heart disease could be treated using tissue from the heart wall and heart valves as source tissue to repair damage to the recipient heart wall and valves. In another embodiment, coronary heart disease could be treated using tissue from the blood vessels of the heart or other vascular structures as source tissue to repair disease of the blood vessels supplying the heart wall. Such treatment could be complemented with the use of heart wall tissue as source tissue to repair any resident heart wall damage. In another embodiment, congenital heart disease caused by genetic factors or by adverse exposures during gestation e.g. holes in the heart, abnormality of valves, abnormal heart chambers could be treated with tissue from the heart wall septum structures, valvular structures or specific chambers of the heart as source tissue to repair or correct the defect in the corresponding structure in the recipient. In another embodiment, peripheral arterial disease may be treated using tissue from arteries as source tissue to repair arterial structures in the recipient.

iii) Respiratory System

Tissues of the respiratory system may constitute the target tissue in various disorders of this system. Examples of disorders of the respiratory system that may be treated by the methods and medicaments of the present invention include damage, pathology, ageing and trauma of the nose and paranasal sinuses, nasopharynx, oropharynx, laryngopharynx, larynx, vocal ligaments, vocal cords, vestibular folds, glottis, epiglottis, trachea, mucocilliary mucosa, trachealis muscle, primary bronchi, lobar bronchi, segmental bronchi, terminal bronchioles, respiratory zone structures and plural membranes. Examples of such damage include obstructive pulmonary diseases, restrictive disorders, emphysema, chronic bronchitis, pulmonary infections, asthma, tuberculosis, genetic disorders (e.g. cystic fibrosis), gas exchange problems, burns, barotraumas and disorders affecting blood supply to the respiratory system. Methods and medicaments of the present invention may also be used to repair, modify, enhance or regenerate the respiratory system following damage. Moreover, methods and medicaments of the present invention may be used as a technique to enhance the transplantation/implantation of whole respiratory structures or organs or their parts.

Examples of source tissue for the treatment of such disorders of the respiratory system includes the entire respiratory system and its whole parts or sub-components, such as nasopharynx, oropharynx or laryngopharynx, larynx, vocal ligaments, vocal cords, vestibular folds, glottis, epiglottis, trachea, mucocilliary mucosa, hyaline cartilages, arytenoids cartilage, cuneiform cartilage, corniculate cartilage, respiratory mucosa, submucosa, adventis, primary bronchi, lobar bronchi, segmental bronchi, terminal bronchioles, respiratory zone structures and plural membranes. In some embodiments, source tissue may comprise individual cell types or cultures enriched for specific cell types. Examples of such cell types include mucosal epithelial cells, pseudostratified columnar, columnar, cuboidal epithelial cells, smooth muscle, type I alveolar cells, type II alveolar cells, alveolar macrophages, pulmonary capillary, diaphragm tissues and intercostals muscle.

In a specific embodiment, cystic fibrosis may be treated with the use of tracheal lining mucosa as source tissue. Such a treatment may ameliorate the respiratory problems and damage associated with this condition. The treatment could be supplemented by the use of GI tract mucosa, thymus, thyroid and/or epithelial tissue of the reproductive tract as source tissue for RNA to target specific deficits in function associated with the condition. In another specific embodiment, cystic fibrosis is treated with the use of cultured endoderm tissue as source tissue. In another specific embodiment, disease or damage of the vocal cords or vocal ligaments could be repaired in the recipient using tissue from vocal cord or ligament structures as source tissue.

### iv) Gastrointestinal Tract and Associated Glands

Tissues of the gastrointestinal tract (and associated glands) may constitute the target tissue in various disorders of the gastrointestinal tract. Examples of disorders of the gastrointestinal tract and associated glands that may be treated by the methods and medicaments of the present invention include disorders, damage and age related changes of both the gastrointestinal tract and the large accessory glands (liver and pancreas), salivary glands, mouth, teeth, oesophagus, stomach, duodenum, jejunum, ileum, ascending colon, transverse colon, descending colon, sigmoid colon, rectum and anal canal and enteric nervous system of the canal. In specific embodiments, these disorders, damage and age related changes include dental caries, periodontal disease, deglutition problems, ulcers, enzymatic disturbances/deficiencies, motility problems, paralysis, dysfunction of absorption or absorptive surfaces, diverticulosis, inflammatory bowel problems, hepatitis, cirrhosis and portal hypertension. Methods and medicaments of the present invention may also be used to repair, modify, enhance or regenerate the gastrointestinal tract following damage, or be used as a technique to enhance any of these processes following surgery, such as resection of the stomach, ileostomy and reconstructive surgery (e.g. ileoanal juncture). Examples of this latter embodiment include reconstructive surgery involving specific anatomical structures of the mouth, such as labia, vestibule, oral cavity proper, red margin, labial frenulum, hard palate palatine bones, soft palate, uvula, tongue, intrinsic muscles of the tongue and extrinsic muscles of the tongue.

Source tissue for the treatment of such disorders of the gastrointestinal tract and associated glands may include the entire gastrointestinal tract and its whole parts or sub-components, including the large accessory glands (liver and pancreas), salivary glands, mouth, oesophagus, stomach, duodenum, jejunum, ileum, ascending colon, transverse colon, descending colon, sigmoid colon, rectum and anal canal and enteric nervous system. Examples of other suitable source tissue include specific tissues or cells defined by structure or function such as mucosal tissues and glands, submucosa, submucosal glands, myenteric nerve plexus, submucosal nerve plexus, musclularis, serosa and specific glands of the digestive tract. In some specific embodiments, the source tissue may be obtained from specific oral structures to include labia, vestibule, oral cavity proper, red margin, labial frenulum, hard palate palatine bones, soft palate, uvula, tongue, intrinsic muscles of the tongue, extrinsic muscles of the tongue. In certain embodiments, source tissue may comprise individual cell types or cultures enriched for specific cell types.

In a specific embodiment, diverticulosis may be treated with the use of tissue obtained from the whole colon or tissue extracted from muscularis and mucosa.

### iv) Integumentary System

Tissues of the integumentary system may constitute the target tissue in various disorders of the integumentary system. Examples of disorders of the integumentary system that may be treated by the methods and medicaments of the present invention include disorders, damage and age related changes of the skin and integumentary system, such as age related decline in thickness or function, disorders of sweat gland and sebaceous glands, piloerectile dysfunction, follicular problems, hair loss, epidermal disease, diseases of the dermis or hypodermis, burns, ulcers, sores and infections. Methods and medicaments of the present invention may also be used to enhance, regenerate or repair skin structures or functions, for example in plastic reconstruction, cosmetic repair, tattoo removal, wound healing, modulation of wrinkles and in the treatment of striae, seborrhoea, rosacea, port wine stains, skin colour and the improvement of blood supply to the skin. Moreover, methods and medicaments of the present invention may be used to enhance skin grafts, surgical reconstruction, cosmetic surgical procedures, wound healing and cosmetic appearance. Source tissue for the treatment of such disorders of the integumentary system includes whole skin, cultured skin, components of the integumentary system, the epidermis, dermis, hypodermis, subcutaneous fats, hair follicles, glands and associated structures. In some embodiments, source tissue may comprise individual cell types or cultures enriched for specific cell types.

In a specific embodiment, full thickness burns may be treated with the use of whole skin as source tissue for the RNA. In another specific embodiment, age related changes or pathology of the skin may be treated using whole skin from a donor of lesser age or developmental stage as source tissue. In another embodiment, age related skin damage could be repaired in a recipient using stem cells as source tissue. In another embodiment age related skin damage in a patient may be ameliorated using fibroblasts, more specifically fibroblasts from a younger donor or from an earlier developmental stage as source tissue. In another specific embodiment, hair loss may be treated in a recipient using hair follicle-associated stem cells as source tissue. In a further specific embodiment, hair loss may be treated using tissue obtained from other types of identified stem cells e.g. mesenchymal stem cells, embryonic stem cells, foetal stem cells, umbilical cord stem cells as source tissue.

### v) Musculoskeletal System

Tissues of the musculoskeletal system may constitute the target tissue in various disorders of the musculoskeletal system. Examples of disorders of the musculoskeletal system that may be treated by the methods and medicaments of the present invention include disease, damage and age related changes of the musculoskeletal system. In some embodiment, these may be in components of the axial skeleton, including the skull, cranium, face, skull associated bones, auditory ossicles, hyoid bone, sternum, ribs, vertebrae, sacrum and coccyx. In other embodiments they may be in components of the appendicular skeleton, including the clavicle, scapula, humerus, radius, ulna, carpal bones, metacarpal bones, phalanges (proximal, middle, distal), pelvic girdle, femur, patella, tibia, fibula, tarsal bones and metatarsal bones. Methods and medicaments of the present invention may also be used to correct problems associated with ossification and osteogenesis, such as intramembranous ossification, endochondral ossification, bone remodelling and repair, osteoporosis, osteomalacia, rickets, pagets disease, rheumatism and arthritis. Moreover, methods and medicaments of the present invention may be used to treat disease, damage and age related changes of the of skeletal muscle, elastic cartilages, fibrocartilages, long bones, short bones flat bones and irregular bones.

Examples of source tissue for the treatment of such disorders of the musculoskeletal system include whole musculoskeletal system and its sub-components, such as specific bones or muscle-derived tissues. In some embodiments, source tissue may comprise individual cell types or cultures enriched for specific cell types. Examples of such cell types include individual musculoskeletal cell types.

In a specific embodiment, damage associated with bursitis may be repaired with the use of synovial membrane as source tissue for the RNA. In another specific embodiment, arthritis may be treated with the use of tissue from articular cartilage or chondrocytes associated with articular cartilage tissueas source tissue. In some embodiments, the source tissue may be from a younger donor than the recipient to change the proteoglycan synthesised by the recipient tissues, thereby reducing the effect of age on chondrocyte generated structures. In another specific embodiment, osteoporosis may be treated using tissue from osteoblasts as source tissue to increase bone density and health in the recipient. In some embodiments, the source tissue may be from a younger donor than the recipient.

### vi) Other Systems of the Body

Disorders of other systems of the body may be treated by the methods and medicaments of the present invention. For example, the present invention may be used to enhance function or treat disease, damage and age related changes in other systems of the body, including special senses, endocrine system, lymphatic system, urinary system, reproductive system and alterations in metabolism and energetics.

Source tissue for the treatment of such disorders may be obtained from whole organs, sub-components of organs, specific cell types and groups of cells, wherein said source tissue is related to the relevant target tissue, as discussed above.

In specific embodiments, damage, disease or dysfunction of any endocrine gland may be repaired using the corresponding gland or its component tissues as source tissue. In a specific embodiment, Type 1 diabetes could be treated using beta cells of the pancreas as source tissue. In other embodiments, where the pathology requires control of blood sugar, appropriate Alpha cells, Delta cells and/or F cells may be used as source tissue to correct dysfunction in the corresponding cells in the recipient.

vii) Treatment of General Age-Related Degeneration.

Methods and medicaments of the present invention may be used to treat, ameliorate, reduce or compensate for general age-related degeneration. Similarly, methods and medicaments of the present invention can be used to retain youthful functions of the body. Moreover, methods and medicaments of the present invention may be used to treat specific age related system dysfunction, such as cognitive impairment, hearing loss, loss of visual acuity, endocrine imbalances, skeletal changes and loss of reproductive function.

Source tissue for such uses may be obtained from whole organs, groups of organs, sub components of such organ(s) and groups of cell types, wherein said source tissue is related to the relevant target tissue, as discussed above.

viii) Enhancement of Specific Physiological Functions

Methods and medicaments of the present invention may be used to enhance the function of specific systems of the body. Accordingly, the present invention may be used to enhance cognitive, cosmetic, anatomical and physical properties of the subject.

Source tissue for such uses may be obtained from whole organ(s), sub-components of organ(s), specific cell type(s) and groups of cells, wherein said source tissue is related to the relevant target tissue, as discussed above. For example, where the desire is to increase muscle mass to increase athletic ability, the source tissue may be muscle tissue, and preferably muscle tissue of the type in which the increase in muscle mass is desired. In another example, where the desire is to increase cognitive ability, the source tissue may be brain tissue. In yet another example, where the desire is to improve blood perfusion to a tissue with a compromised blood supply, the source tissue may be vascular tissue.

### Preparation of RNA

Various techniques exist for the extraction of donor RNA for use in the methods and medicaments of the present invention. Such techniques may be used to obtain the RNA to be provided to the target cells. Alternatively, such techniques may be used to provide RNA to identify the sequences of the necessary RNA molecules in the RNA extract (e.g. by fractionation and screening). Thus the invention includes a method of screening for an RNA sequence capable of conferring a desired property from one cell type to another, comprising the steps of:
i. extracting RNA from cells comprising a desired cell type;
ii. separating the extracted RNA into different fractions;
iii. providing a fraction to one or more test cells and/or test recipients;
iv. analysing the test cells or recipients for an altered property possessed by the desired cell type from which the RNA was extracted;
   wherein a fraction that confers the altered property onto a test cell or recipient is identified as comprising an RNA sequence capable of conferring the desired property.

This screening method identifies RNA sequences that are capable of conferring a desired property from one cell type to another by fractionating the RNA extract and analysing RNA function using an appropriate assay. One example of an appropriate assay is an experiment of the type described in Example 1 below. The assay comprises providing isolated RNA comprising RNA extractable from cells comprising particular cell type(s) to a population of cells; and determining whether a cell property is altered towards a property of said desired cell type(s). In this way, RNA in an extract can be identified as unnecessary for the purposes of the invention and can be omitted (e.g. to simplify or standardise an RNA composition), ultimately leaving an RNA molecule, or set of RNA molecules, which are responsible for the desired activity.

Accordingly, the present invention also envisages the use of specific RNA sequences, specific RNA subtypes, or particular RNA structures that have been identified as capable of conferring a desired property from one cell type to another in the RNA extract. Examples of suitable RNA molecules include messenger RNA (mRNA), ribosomal RNA (rRNA), transfer RNA (tRNA), heterogeneous nuclear RNA (hnRNA), small nuclear RNA (snRNA), small cytoplasmic RNA (scRNA), small nucleolar RNA (snoRNA), transcription-related RNAs, splicing-related RNAs, signal recognition particle RNAs, linear RNA, circular RNA, inhibitory RNA (e.g. siRNA), single-stranded RNA, double-stranded RNA, and so on. Such RNA molecules may be synthesised artificially. In some cases, the RNA may be an artificial or synthetic RNA or an RNA analogue based on the sequence of the extractable sequences. The analogue may be one chosen for its stability or ability to enter the target cell or other desirable properties.

In some embodiments, the RNA employed in the invention will be one that comprises RNA sequence extractable from tissues or cells comprising the cell type or types that it is desired to induce the target cell to have a cell property in common with. Thus, in the case where the aim is, for example, to induce differentiation of a stem cell into a desired differentiated cell type, the RNA provided to the target cell may typically be an isolated RNA comprising RNA sequence extractable from tissue or cells comprising the desired differentiated cell type or types.

Suitable techniques include preparation by either cold or hot phenol extraction methodologies. Alternatively, the RNA may be sourced from specific tissues or cells by employing commercially available kits and in particular those that are based on the denaturing of protein and separation of RNA via centrifugation. For example, in one preferred protocol (cold phenol) extraction, primary donor tissue or cells is/are homogenised in a volume of physiological saline. An equal volume of 95% saturated phenol is added and initially centrifuged at 18,000 rpm in an ultra-centrifuge for 30 minutes. The aqueous phase is retained and brought to a concentration of 0.1M MgCl₂ solution by the addition of 1M MgCl₂. Two volumes of ethanol are then added and this is allowed to precipitate for approximately 30 minutes. A final spin at 6,000 rpm for 15 minutes produces an RNA rich precipitate which can be retained and stored under ethanol. Alternatively, active RNA rich extracts may be prepared with any of the commercially available RNA extraction kits (such as, for example, RNAzol™). However, the precise methodology by which the RNA is extracted is generally not critical to the invention.

In some embodiments, the RNA used in the methods and medicaments of the invention will comprise “total RNA”, that is, a crude extract of RNA resulting from the extraction of essentially all types of RNA from the source tissue.

In some cases a specific fraction of an RNA extract may be employed. For example, the RNA population may be fractionated on the basis of size and a particular weight range of RNA species provided to the target cell. Fractionation may also be on the basis of weight, charge, or identifiable common chemical feature (for example, a structure, or the presence of a particular consensus or pattern of nucleotides) or any combination of size, weight or charge or common chemical feature.

In particular, the present inventors have ascertained that the active fraction of total RNA that is effective to impart the beneficial effects of the inventions as described herein is the polyA positive fraction (i.e. the fraction of RNA that is polyadenylated). Accordingly, in preferred embodiments, the RNA used in the present invention is isolated polyA positive RNA in substantially pure form. By “substantially pure” is meant that the RNA consists essentially of isolated polyA positive RNA. However, as fractionation techniques based on the polyadenylation status of the RNA may not be 100% efficient, the RNA fraction may comprise a residual amount of polyA negative RNA. Isolated polyA positive RNA may be obtained from total RNA using any suitable fractionation techniques known in the art, for example as described above and in Aviv et al (1972) PNAS 69, 1408-1412, Sambrook and Russell (2001) Molecular Cloning. A Laboratory Manual (3^{rd} ed.) Cold Spring Harbor Laboratory Press, Cold Spring Harbor, N.Y. and reviewed by Hornes and Korsnes (1990) Genet. Anal. Tech. Appl. 7: 145-150 and Jarret (1993) J. Chromatogr. 618: 315-339. Suitable methods include those that isolate polyA positive RNA by hybridisation of the polyA tail to a thymine oligomer coupled to a solid-phase matrix. In particular, isolated polyA positive RNA may be obtained using a commercially available separation kit, such as the Poly(A) Purist™ mRNA purification kit (Ambion cat #1916, see Ito et al (2003) Am J Path, 163, 2165-2172); the Poly(A) Purist™ MAG magnetic poly(A) RNA purification kit (Ambion cat #1922, see Hyun et al (2004) Mol Cell Biol 24, 4329-4340); or the μMACS™ mRNA isolation kit (Miltenyi Biotec cat #130-075-102, see Fischer et al (2002) J Neuroscience, 22, 3700-3707).

In particular, polyA positive RNA may be prepared according to the following protocol:

An extract of total RNA (comprising no more than 2,000 μg RNA) is resuspended in 0.75 cm³ nuclease free water and vortexed to resuspend the pellet. An equal volume of 2× binding solution (Poly(A) Purist™ mRNA purification kit, manufacturer's protocol) is added and mixed thoroughly. Each RNA sample is then added to a tube containing 100 mg oligo(dT) cellulose and mixed by inversion. The resultant mixture is then heated to 70° C. in a water bath for 5 minutes. After this time, the mixture is agitated gently for 60 minutes at room temperature. The oligo(dT) cellulose is pelleted by centrifuging the mixture at 3000 g for 3 minutes at room temperature. The resultant supernatant (which contains the polyA negative RNA) is then removed by aspiration and discarded.

In a washing step, 0.5 cm³ of Wash Solution 1 (Poly(A) Purist™ mRNA purification kit, manufacturer's protocol) is added to the oligo(dT) cellulose pellet and the mixture vortexed to resuspend the pellet. A spin column is placed in a 2 ml microfuge tube and the oligo(dT) cellulose suspension transferred to this column, which is then centrifuged at 3000 g for 3 minutes at room temperature. The filtrate is discarded from the microfuge tube and the spin column returned to the tube. This washing step is repeated a further time with Wash Solution 1 and a further three times with Wash Solution 2 (Poly(A) Purist™ mRNA purification kit, manufacturer's protocol).

The spin column is then placed in a fresh microfuge tube and 200 μl of warm THE RNA Storage Solution (Ambion cat #7001) (previously heated to 70° C. in a water bath) added to the oligo(dT) cellulose pellet. The mixture is vortexed briefly to mix the two and the tube immediately centrifuged at 5,000 g for 2 minutes at room temperature. This addition of warm THE RNA Storage Solution is repeated a further two times.

The spin column is discarded and 40 μl 5M ammonium acetate, 1 μl glycogen and 1.1 ml 100% ethanol added to the filtrate. This mixture (which contains the polyA positive RNA) is then stored at −70° C. for 30 minutes.

To recover the polyA positive RNA, the mixture is centrifuged at 12,000 g for 30 minutes at 4° C. and the supernatant removed by aspiration and discarded. The remaining pellet is then washed with 70% ethanol and vortexed. Finally, a polyA positive RNA pellet is obtained by centrifuging the resultant mixture at 12,000 g for 10 minutes at 4° C. This sample may be stored at −20° C. until required.

Accordingly, in some embodiments, the RNA will be fractionated total RNA. This may be obtained by one or more RNA fractionation techniques, as described in the following list. These techniques may be applied sequentially, each step involving the retention/removal of particular RNA fractions. Sometimes, it will be appropriate to pool fractions in a defined ratio before carrying out a further fractionation technique or in the production of the final RNA preparation for use in the methods and medicaments of the invention.

### Fractionation Techniques:

1) Fractionation according to the presence or absence of polyadenylation, for example as described above.
2) Fractionation according to mobility, for example by any suitable electrophoresis technique, for example as described in Pley et al. (1993) J. Biol. Chem. 268: 19656-19658 and Heus et al. (1990) Nucleic Acids Res. 18: 1103-1108.
3) Fractionation according to density, for example by any suitable centrifugation technique such as by sucrose gradient fractionation, described in Jain et al. (1997) Mol. Cell. Biology 17(2): 954-962.
4) Fractionation according to binding characteristics, for example by any suitable affinity purification technique such as described in Schnapp et al. (1998 Nucleic Acids Res. 26(13): 3311-3313.
5) Fractionation according to mass/size, for example by any suitable chromatography technique, as described in Lee & Marshall (1986) Prep. Biochem. 16(3): 247-58.
6) Fractionation according to the presence or absence of bound protein, for example by any suitable purification technique (e.g. immunological) that recognises the bound protein.
7) Fractionation according to inherent sequence information. The removal or enrichment of RNAs with particular sequence properties is specifically envisaged in the preparation of RNA for use in the present invention. For example, in some embodiments, it will be desirable to modify the content of the RNA extract by the selective removal or enrichment of RNAs of particular sequence. The selective removal or enrichment of RNAs of particular sequence may be achieved using any suitable complementary sequence RNA separation technique (for example, as described in Srisawat et al (2001) Nucleic Acids Res 29, E4).

In particular, the present invention provides a method for isolating from an RNA extract a fraction of RNA molecules that comprise a specific sequence. This method comprises the steps of:
i) contacting the RNA extract with one or more nucleic acid species capable of annealing to an RNA fraction in the extract;
ii) incubating the resultant mixture under conditions whereby said one or more nucleic acid species anneal with said fraction; and
iii) isolating the annealed fraction from the remainder of the extract, wherein said fraction is the fraction of RNA molecules that comprise the specific sequence.

The expression “nucleic acid” in step i) above typically means RNA, preferably synthetic RNA. However, in some embodiments, it may mean DNA, including cDNA, synthetic DNA or genomic DNA. The term “nucleic acid” also includes analogues of DNA and RNA, such as those containing modified backbones, and peptide nucleic acids (PNA).

Preferably, the one or more nucleic acid species of step i) are single-stranded, although in some embodiments double-stranded nucleic acids may be used.

In order to be capable of annealing to an RNA fraction in the extract, the nucleic acid species will typically comprise sequence that is complementary to a specific sequence found in the molecules of the RNA fraction to which they are to anneal. It will be appreciated that absolute complementarity, although preferred, may not be required for the species to anneal to the RNA fraction of interest. Accordingly, in some embodiments, the species may comprise sequence that is 99, 95, 90, 85, 80, 75, 70, 65, 60, 55 or 50% complementary to the specific sequence found in the RNA fraction of interest, provided that said level of complementarity allows the species to anneal with said molecules under the conditions used.

Generally, the sequence to which the species are complementary is only part of the sequence of the RNA molecules in the target fraction. Accordingly, the species need not be of the same length as the RNA molecules in the fraction. However, the species must be of an appropriate length to anneal selectively to RNA molecules comprising the complementary sequence. Designing suitable species for any given target RNA fraction would be routine to those skilled in the art. In general, the one or more nucleic acid species of step (i) are between 10 to 500 bases long. More preferably, they are between 10 to 250; 10 to 150; 10 to 100; 10 to 90; 10 to 80; 10 to 70; 10 to 60; 10 to 50; 15 to 40; 15 to 30 or 15 to 25 bases long. Even more preferably the one or more nucleic acid species are 17 to 25 bases long. More preferably, the one or more nucleic acid species are 20 bases long. Where more than one nucleic acid species is used, each species may be of a uniform length or they may have lengths that are independent of the lengths of the other species used. Preferably, they are of uniform length.

In some embodiments, the annealed fraction may comprise the RNA fraction for use in the present invention and will therefore be retained. However, in other embodiments, the non-annealed fraction will comprise the fraction for use in the present invention and the annealed fraction will therefore be discarded.

In a preferred embodiment, selective isolation of the annealed fraction is achieved as follows. The one or more nucleic acid species in step i) are coupled to one or more groups that are suitable for isolating the annealed fraction from the non-annealed fraction in the isolation of step iii). Examples of suitable groups include, but are not limited to, any tag that may be used for the purification of nucleic acids, such as biotin or a specific nucleic acid sequence. Nucleic acid fractions comprising such tags may be separated by contacting the fraction with (strep)avidin or a complementary nucleic acid sequence respectively. Those skilled in the art will be aware of other suitable tags and separation techniques for use in this embodiment.

This method for isolating from an RNA extract a fraction of RNA molecules that comprise a specific sequence may be used to remove RNA sequences that induce specific genotypic modifications in cells of the target tissue (see co-pending UK Patent Application, Agent's Reference G039766PT). The remaining RNA may be used in the present invention. Accordingly, in some embodiments, the RNA used in the present invention may lack RNA sequences that have the ability to induce one or more specific genotypic modifications in the target cells (for example, to the extent that the RNA used is substantially free of such sequences). This selective removal of RNAs of particular sequence may be desired when the unfractionated RNA comprises RNA sequences with the ability to induce one or more unwanted genotypic modifications in the target cells. For example, if the RNA is derived from a subject with a specific genetic mutation and it is desired for these mutations not to be induced in the target cells, RNA sequences capable of inducing the mutation may be removed. In another example, RNA sequences capable of inducing genotypic modifications to the genes defining one or more histocompatibility elements, e.g. MHCI, MHCII or other immune response-inducing proteins, may be removed.

The selective removal of RNA molecules responsible for specific modifications may be achieved using the method described above, wherein the one or more nucleic acid species of step (i) comprise sequence that is capable of annealing with the RNA molecules responsible for the specific modifications. Accordingly, the one or more nucleic acid species of step (i) will typically comprise sequence that is complementary to sequence in the RNA molecules responsible for the specific modifications.

Where the sequence of the RNA molecules is unknown, it may be possible to design suitable species on the basis of the genomic modification that the molecules cause. Without wishing to be bound by theory, it is believed that an RNA molecule responsible for a specific modification will comprise one or more regions that are complementary to a sequence in one of the strands of DNA at the genomic region that is modified. The one or more regions that are complementary to a sequence in one of the strands of DNA at the genomic region that is modified may be complementary to the coding strand of the DNA. Alternatively, these one or more regions may be complementary to the non-coding strand of the DNA.

Accordingly, the one or more nucleic acid species of step (i) may comprise sequence that is complementary to a sequence of DNA at the genomic region in the source tissue corresponding to the genomic region modified in the target cells. The sequence that is complementary to a sequence of DNA at the genomic region in the source tissue corresponding to the genomic region modified in the target cells may be complementary to the coding strand of the DNA. Alternatively, this sequence may be complementary to the non-coding strand of the DNA.

Alternatively, in other embodiments, the species may comprise sequence that is complementary to a sequence of DNA at a genomic region some distance away from this region. For example, the complementarity may be to a strand at a region that is 10, 20, 30, 40, 50, 60, 70, 80, 90, 100, 200, 300, 400 or 500 bases away (upstream or downstream) from this region. In some cases, this may result in the species annealing with RNA molecules that do not comprise sequence complementary to the genomic region in the source tissue corresponding to the genomic region modified in the target cells. Instead, they may comprise sequence complementary to regions that flank said genomic region. Such molecules are not thought to be capable of effecting the specific genotypic modification when used alone. However, as discussed below, these molecules may be useful for improving the efficiency of genomic modification when used in combination with RNA molecules that comprise sequence complementary to the genomic region concerned.

As noted above, in some embodiments, the species used in step i) will be double stranded nucleic acid. Accordingly, in such embodiments, the species may comprise two strands, one complementary to one strand of DNA at the genomic region concerned and the other complementary to the other strand of DNA at the genomic region concerned. Such species will therefore comprise sequence that is complementary to the coding strand of the DNA and the non-coding strand of the DNA.

In some embodiments, more than one species may be used in step i). This may improve the efficiency of the isolation of specific RNA molecules. In such embodiments, each different species may comprise sequence that is complementary to adjacent sequences in the DNA at the genomic region concerned.

Where the specific genotypic modification of interest involves the alteration of only a few bases (e.g. less than 20 bases), a single nucleic acid species may be used in step (i), for example a single species of 17 to 25 bases, more preferably 20 bases.

In some embodiments, the specific genotypic modification of interest may involve alteration to a genomic region that is relatively long, for example between 100 and 500, 500 and 1000, 1000 and 5 000, 5 000 and 10 000, 10 000 and 100 000 or 100 000 and 1 000 000 bases long. This may be the case when the modification involves the substitution or insertion of an exon or entire gene from the source tissue. In such embodiments, it is preferred to use more than one species in step i). For example, multiple species of 17 to 25 bases, more preferably 20 bases may be used. Typically, each different species comprises sequence that is complementary to adjacent sequence in the DNA at the genomic region concerned. These adjacent sequences may be spaced along the length of the genomic region concerned in order to reduce the number of species required. The optimal spacing for a given RNA target may be determined by routine experimentation. However, typical spacings would be between 100 and 1000 bases. The spacings may be uniform (i.e. all of the same length) or non-uniform.

In some embodiments, the species used in step i) may include one or more species comprising sequence that is complementary to a sequence of DNA at the genomic region in the source tissue corresponding to the genomic region modified in the target cells and one or more species comprising sequence that is complementary to a sequence of DNA that is at a genomic region some distance away from this region. This may result in the various species used in step i) annealing with multiple different RNA molecules in the RNA extract. As noted above, some of these RNA molecules may not comprise sequence that is complementary to the genomic region in the source tissue corresponding to the genomic region modified in the target cells. Despite this, the presence of these RNA molecules may improve the efficiency of genotypic modification. In particular, where the genotypic modification involves the insertion of a sequence that is not present in the target tissue, such RNA molecules may be essential for successful modification.

The sequence chosen for the species used in step i) may be used to probe the genome of the source tissue in silico to ensure that it is complementary to DNA at one genomic region. Preferably, species that are complementary to DNA at only one genomic region in the source tissue will be used in step i). This will limit the extent of non-specific binding to other RNA molecules that may be present in the RNA extract.

The number of different species required and the specific regions of DNA to which they are complementary may be determined for any given genotypic modification by routine experimentation.

Although the inventors have ascertained that the fraction of total RNA responsible for the effects of the present invention (i.e. the “active” fraction) is the polyA positive fraction, in some embodiments of it may be desirable to further fractionate the RNA so that an even more pure sample of active RNA is obtained. This may be particularly the case in the methods of treatment and medicament of the present invention, where for regulatory reasons, inter alia, it may be necessary to administer a more defined preparation of RNA. Accordingly, in some embodiments, the RNA will have been further fractionated to concentrate the active fraction, for example by fractionating the RNA and carrying out a functional assay on the various fractions to identify the active fraction at each stage of the fractionation.

Accordingly, the RNA used in the present invention will preferably be one that is obtainable by the following procedure:
a) extracting RNA from source tissue;
b) separating the extracted RNA into different fractions;
c) providing each fraction to separate samples of one or more test cells;
d) analysing the test cells for an altered property possessed by the source tissue from which the RNA was extracted; and
e) retaining the fraction that results in an altered property in the test cells.

This procedure identifies RNA sequences that are capable of conferring a desired property from one cell type to another by fractionating the RNA extract and analysing RNA function using an appropriate assay. Any one or more suitable fractionation techniques from the list given above may be used in this procedure. Moreover, an example of an appropriate assay for use in this procedure is an experiment of the type described in Example 1 below. The assay comprises providing isolated RNA comprising RNA extractable from cells comprising particular cell type(s) to a population of cells; and determining whether a cell property is altered towards a property of said desired cell type(s).

Alternatively, the RNA used in the present invention will preferably be one that is obtainable by the following procedure:
a) extracting RNA from source tissue;
b) separating the extracted RNA into different fractions;
c) using each fraction as the RNA in a treatment of the present invention;
d) analysing the test recipients for a treatment response; and
e) retaining the fraction that results in a treatment response in the test recipient.

This procedure identifies RNA sequences that are capable of effecting in a treatment response in the test recipient by fractionating the RNA extract and analysing RNA function using an appropriate treatment. Any one or more suitable fractionation techniques from the list given above may be used in this procedure. Moreover, an example of an appropriate assay for use in this procedure is an experiment of the type described in Example 11 below. The treatment comprises providing isolated RNA comprising RNA extractable from a source tissue to a subject and determining whether a treatment response is effected.

In some embodiments the RNA fraction or RNA molecule may be specific to affecting some parts of the RNA-treatment effect but not others. For example, one RNA type or molecule may only effect migration but not have other effects such as migration, terminal differentiation, integration or proliferation. In another embodiment, the RNA type or molecule may affect all factors other than migration. In another embodiment, the RNA type or molecule may effect only the degree of proliferation, or only the phenotypic cell type of terminal differentiation, but not any other aspect. In some cases the RNA may comprise a mixture of sequences extractable from different cell types or tissues. For example, the RNA species may comprise a mixture of sequences extractable from two, three, four, five or more different cell types. In cases where it is desired to differentiate a stem cell, the RNA may, for example, be extractable from different cell types to produce a differentiated cell with characteristics of both cell types. In cases where the RNA is to be provided to a target cell that has a genetic defect, the RNA may be a mixture of sequences extractable from cells comprising and lacking the defect. For example, the RNA may comprise a blend of RNA extracts from cells from the subject with the defect and cells of the same type from another subject that lack the defect. In some cases specific sequences that are extractable from the desired cell type may not be present. For example, the transcript of a defective gene may be removed. The removal of specific sequences may, for example, be achieved, by selective degradation or by hybridisation. Ribozymes may be used to cleave specific sequences. RNase molecules may also be used with some degree of specificity.

Specific sequences may be added to or removed from the extractable sequences. For example, in some cases the RNA may originate from the subject intended to be the eventual recipient of the medicaments of the present invention and the subject may lack a specific gene sequence or have a defective gene sequence. In such cases an additional RNA corresponding to an RNA encoding the expression product of the missing or defective gene may be added to the extract. In such cases, the targeted genetic sequences may be repaired, modified, removed or selectively degraded.

In cases where the RNA is one extractable from a stem cell, preferred stem cells include any of those mentioned herein and in particular adult stem cells. The stem cell may, for example, be a haematopoietic, bone marrow stromal or neuronal stem cell. In cases where the RNA is one extractable from a differentiated cell, the differentiated cell may be any differentiated cell and may be in particular an adult differentiated cell. In a preferred embodiment the differentiated cell may be selected from a bone marrow cell, a neuronal cell, or a haematopoietic cell. The differentiated cell may be from any mammalian organ for example such as the kidney, liver, heart, pancreas, central nervous system, reproductive organ or other organ.

In some embodiments, isolated RNA extractable from cells and used in the methods of the invention is natural in derivation. By this is meant that the RNA contains no non-natural sequences and entirely consists of RNA from the species to which the cell belongs. In some embodiments, the RNA contains no viral, exogenous retroviral or pathogen sequences. In some embodiments, the RNA is a homogenous mixture and contains no siRNA, miRNA or other types of interfering RNA. In some embodiments, the RNA may not encode protein (e.g. the RNA does not have in-frame start and stop codons flanking a protein-coding region). In some embodiments, the RNA is not extractable from neoplastic cells. In some embodiments, the RNA contains no double-stranded RNA of a kind that directly activates an anti-viral immune response (e.g. by binding to a Toll receptor). In some embodiments, the RNA contains no antisense RNA (e.g. there is no RNA that is complimentary to the sense strand of an RNA transcript that is also present). RNA used according to the invention may be integrating or non-integrating. It may or may not be capable of replication. It may or may not have a 5′ cap. It may or may not act as a substrate for endogenous reverse transcriptase. It may or may not have a specific secondary structure, e.g. a hairpin. It may or may not have catalytic properties (e.g. for DNA, RNA, protein or other bioactive substrate). It may or may not have autocatalytic properties. It may or may not contain introns. It may or may not act to regulate gene expression. It may or may not contain double-stranded RNA. It may or may not be mRNA. It may or may not have associated protein component. It may or may not work as an enzyme. It may or may not be self-replicating. It may or may not be messenger RNA (mRNA), ribosomal RNA (rRNA), transfer RNA (tRNA), heterogeneous nuclear RNA (hnRNA), small nuclear RNA (snRNA), small cytoplasmic RNA (scRNA), small nucleolar RNA (snoRNA), transcription-related RNAs, microRNAs, splicing-related RNAs, signal recognition particle RNAs, linear RNA, circular RNA, inhibitory RNA (e.g. siRNA), single-stranded RNA and double-stranded RNA. It may or not be riboswitch RNAs (Nature 428:281), X (inactive) specific transcripts (XIST), ribonuclease P RNAs, small modulatory RNAs (Cell, 116:281), Makorin1-p1 RNA (Nature 423:91), Fragile X rGG repeat RNA (Neuron 39:739).

### Modified RNA and Analogs

The invention generally involves the use of RNA. This RNA comprises a sequence that can be extracted from cells comprising a desired characteristic. Transfer of the RNA to a target cell causes desired changes in the target cell, with the changes being defined by the RNA.

As shown herein, the RNA in which the changes are defined is active even when delivered within a phenol extract of RNA from a starting cell. This phenol extract contains a variety of different RNA molecules. If the activity is associated with specific RNA molecules and/or sequences within the extract then, to simplify preparation and quality control, it is preferred to deliver just the specific RNA rather than a complex mixture. The specific RNA can be prepared by purification from the RNA extract, or can instead be prepared synthetically or artificially (e.g. by chemical synthesis, at least in part, or by purification after transcription of the specific RNA from a template nucleic acid).

Accordingly, in addition to the use of RNA obtainable by extraction (optionally including additional fractionation, as discussed above), it is also specifically envisaged to use RNA prepared synthetically or artificially (e.g. by chemical synthesis, at least in part, or by purification after transcription of the specific RNA from a template nucleic acid) in the methods and medicaments of the present invention.

Thus the invention provides a process for preparing an RNA for use with the invention, comprising the step of synthesising the RNA by chemical means, at least in part. The invention also provides a process for preparing an RNA for use with the invention, comprising the steps of: contacting a template for said RNA with an RNA polymerase, whereby the polymerase can interact with the template to produce said RNA. The RNA polymerase could be an RNA-dependent RNA polymerase, but will typically be a DNA-dependent RNA polymerase (i.e. the template is preferably DNA, e.g. in the form of a plasmid).

The RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine which are not as easily recognised by endogenous endonucleases. Bases such as pseudo-uridine, methyl-cytosine, and inosine may be present in such RNA molecules. It is also possible to include DNA nucleotides to form a DNA/RNA chimera. The use of modified backbones is a preferred feature of modified RNA molecules of the invention.

RNA analogs and mimics can also be used. Polymers that mimic natural RNA structures can be prepared and used with the invention etc. e.g. as described by Kirshenbaum et al. (1999). These modified molecules and analogs can be considered as “RNA” herein even if, from a strict chemical viewpoint, they are not simply ribonucleic acid.

### Amplified RNA

RNA obtainable by extraction (optionally including additional fractionation), as discussed above, or RNA prepared synthetically or artificially, as discussed above, may be amplified in vitro to increase the amount of active RNA present. Suitable techniques for this amplification, such as in vitro expansion of arbitrary RNA sequences, would be well known to those of skill in the art. For example, RNA may be amplified using the BD SMART™ mRNA amplification technique (Chenchik, A., et al. (1998) Generation and use of high-quality cDNA from small amounts of total RNA by SMART PCR, Gene Cloning and Analysis by RT-PCR (BioTechniques Books, MA), pp. 305-319).

### Provision of RNA to Target Cells

The RNA may be provided to the target cells in vitro or in vivo. The RNA may also be used in the manufacture of medicaments for the provision of the RNA to the target cells in situ. This is particularly the case where the RNA is provided to target cells in the human body. The RNA may be provided to the target cells by any suitable technique.

A number of methods for the provision of nucleic acid molecules to cells are known and these may be employed. For example, suitable techniques may include calcium phosphate transfection, DEAE-Dextran, electroporation, liposome encapsulation, liposome-mediated transfection, microsphere encapsulation, transduction using viral envelope particles and microinjection. The calcium phosphate precipitation method of Graham & van der Eb (1978) may be employed. General aspects of mammalian cell host system transformations have been described in U.S. Pat. No. 4,399,216 and may be employed. For various techniques for transforming mammalian cells, see Keown et al. (1990) and Mansour et al. (1988). In some cases the RNA or the enclosed RNA may be bound to chemical agents that enhance uptake by the target cells. For example the RNA of RNA-containing particles may be linked to an antibody specific to an appropriate receptor. Such a targeting chemical may increase uptake by all cell types, or may have an effect which is specific to a particular cell type or stem cell type. As an alternative, RNA can be administered without being bound to such reagents e.g. naked RNA. In some cases the RNA may simply be added to the culture medium of the cells for a suitable period of time. For example, the cells and RNA may be cultured together for from 1 minute to 10 days, preferably from 1 hour to 5 days, more preferably from 6 hours to 2 days. In a preferred embodiment the RNA may be cultured with the cells for 12 or 24 hours and in particular for 12 hours. In another example, the cells and RNA may be cultured together for prolonged periods from 10 days to 60 days. In a preferred embodiment, the RNA may be cultured with the cells for 10 to 14 days. In a further example, the RNA may be repeatedly administered to the cells hourly, daily or weekly. In another preferred embodiment, the RNA may be cultured with the cells for a short duration, for example from 1 minute to 6 hours and in particular for 1 hour. Similar time periods may be employed where the RNA is provided in the form of liposomes comprising the RNA sequences or by any of the other methods for providing RNA outlined above.

In some embodiments, the temperature of the cells in culture may be lowered or raised to facilitate uptake of the RNA. The cells are typically maintained at a constant pH. In some embodiments, the cells may be osmotically shocked to facilitate RNA uptake. The culture conditions may contain specified serum, or may be serum free. In some embodiments, the media may be conditioned by specific tissues or cell types. In other examples, the cells may be grown on a defined substrate (e.g. gelatin, polylysine, feeder cell layer etc.)

The amount of RNA provided to the target cells will be sufficient to bring about the necessary desired alteration in a cell property. For example the concentration of RNA may be from 10 ng to 5 mg/ml, preferably from 100 ng/ml to 2.5 mg/ml, more preferably from 1 μg/ml to 500 μg/ml, even more preferably from 5 μg/ml to 100 μg/ml and still more preferably from 10 to 50 μg/ml. In a particularly preferred case the RNA concentration may be from 15 to 40 μg/ml, preferably from 20 to 35 μg/ml and in particular may be 25 μg/ml. These concentrations may apply to in vitro or in vivo applications. In some cases, a total of 100 ng to 0.1 g, preferably from 1 μg to 50 mg, more preferably from 100 μg to 10 mg, still more preferably from 250 μg to 1 mg of RNA may be administered. Any suitable concentration and/or amount of RNA may be provided. A wide range of concentrations and/or amounts of RNA may be employed and the precise concentration and/or amount may be varied according to the method of delivery of the RNA to the target cells, the source of the RNA and whether the RNA is provided in vitro or in vivo. Once aware of the teaching of the present invention, it would be routine to the skilled reader to optimise the amount of RNA provided to the target cells in order to bring about the desired alteration.

The response of the stem cells to RNA of the invention may be enhanced by appropriate adjustment of the medium during treatment. For example, the medium may be adjusted to reduce RNA degradation. In one preferred embodiment, the medium is RNase-free. In another preferred embodiment, the medium contains an RNase inhibitor, preferably in saturating doses.

The RNA administered to a subject or used in the ex vivo treatment of stem cells may be extracted RNA as prepared according to any of the methods described above. However, in some embodiments, the RNA is first modified to increase its effectiveness or improve its in vitro and/or in vivo delivery. For example, suitable modifications include one or more of the following techniques.

### i) Microvesicle Packaging

The RNA may be contained in microvesicles (e. g. liposomes), in order to protect the RNA from RNase degradation and/or improve uptake.

Typically, for in vitro use, the composition and other characteristics of the microvesicles (e. g. size, wall thickness etc.) will be chosen to optimise the effective uptake of the RNA by the recipient stem cells (for example, by ensuring that the RNA is taken up in a cellular pathway that results in the desired effect), and/or to minimise degradation of the RNA during incubation. The composition and other characteristics of the microvesicles may also be chosen to facilitate the addition of ligands (described below) to enhance effective uptake by the recipient stem cells.

Typically, for in vivo use, the composition and other characteristics of the microvesicles (e.g. size, wall thickness etc.) will be chosen to optimise the effect of the RNA on the target tissue (for example, by optimising effective uptake by the target tissue and/or minimising uptake by non-target tissue). The composition and other characteristics of the microvesicles may also be chosen to facilitate the addition of ligands (described below) to optimise the effect of the RNA on the target tissue (for example, by optimising effective uptake by the target tissue and/or minimising uptake by non-target tissue).

### ii) Carrier Materials

The RNA may be conjugated or otherwise attached to carriers (e.g. polyethyleneglycols of a particular molecular weight, sugars, lipids (e.g. cholesterol) or proteins) that protect the RNA from degradation, e.g. by RNase, and/or improve effective uptake.

Typically, for in vitro use, the nature of the carriers will be chosen to optimise the effective uptake of the RNA by the recipient stem cell and/or to minimise degradation of the RNA during incubation. Carriers may also be chosen to facilitate the addition of ligands (described below) to enhance effective uptake by the recipient stem cells.

Typically, for in vivo use, the nature of the carriers will be chosen to optimise the effect of the RNA on the target tissue (for example, by optimising effective uptake by the target tissue and/or minimising uptake by non-target tissue). The nature of the carriers may also be chosen to facilitate the addition of ligands (described below) to optimise the effect of the RNA on the target tissue (for example, by optimising effective uptake by the target tissue and/or minimising uptake by non-target tissue).

iii) Ligand Targeting Moieties

The RNA (optionally packaged in microvesicles or attached to a carrier as described above) may be complexed with a ligand that results in selective uptake by the target tissue. For example, the RNA may be linked to an antibody or antibody fragment that targets the RNA to a desired population stem cells and results in effective uptake of the RNA by said cells.

### iv) Stored RNA

In some embodiments, the RNA of the invention may have been stored before use.

In some embodiments the RNA may be used in treatment methods or in the manufacture of medicaments which will allow in vivo provision of the RNA to stem cells or to other cells. In such cases the RNA is typically formulated so that the medicament is in a suitable form for administration to the intended subject.

The medicament may be in a form where the RNA is in liposomes to facilitate delivery or alternatively encapsulated within viral envelope particles. The RNA may be present as naked RNA molecules or RNA molecules complexed with proteins and in particular proteins known to increase uptake of nucleic acids into cells.

The medicament may be administered in conjunction with other treatments given prior, simultaneously or subsequently which increase the time for which the medicament remains in an active state, in vitro or in vivo. For example the use of a known RNase inhibitor could be used for such treatment. Alternatively saturating dose of inactive or sacrificial RNA may be given to block the existing RNase activity.

The medicament may be administered in conjunction with other treatments given systemically or locally, prior, simultaneously or subsequently which increase the uptake or effect of the medicament in vitro or in vivo. For example molecules secreted in a local or systemic manner following tissue damage may enhance uptake of the medicament. Such molecules may originate from the damaged tissue per se, or from a stem cell source. In another example, known non-RNA inducers of tissue differentiation of specific tissues culture may be used in conjunction with the RNA of this method in vitro for example, the use of retinoic acid to aid differentiation of neuronal tissues. In another example known non-RNA supports of tissue culture may be used in conjunction with the medicament, for example, basic fibroblast growth factor in the culture of spinal neurons.

The medicament comprising the RNA may be delivered by any suitable route. For example, the medicament may be administered parenterally and may be delivered by an intravenous, rectal, oral, auricular, intraosseous, intra-arterial, intramuscular, subcutaneous, cutaneous, intradermal, intracranial, intratheccal, intraperitoneal, topical, intrapleural, intra-orbital, intra-cerebrospinal fluid, transdermal, intranasal (or other mucosal), pulmonary, inhalation, or other appropriate administration route. The medicament may be administered directly to the desired organ or tissue or may be administered systemically. In particular preferred routes of administration include via direct organ injection, vascular access, or via intramuscular, intravenous, or subcutaneous routes. The RNA may be formulated in such a way as to facilitate delivery to the target cells.

The RNA may be provided on metallic particles. In some cases the medicament may be intended to be administered so that naked RNA is provided to the target cells. In cases where the RNA is provided present in liposomes or other particles, there may be targeting molecules present on the surface of the particles to allow targeting to the intended stem cells. For example, the particles may comprise ligands for receptors on the target stem cells or target differentiated cells. In one preferred embodiment, RNA is delivered to the cells via liposomes prepared after the methodology of Felgner et al. (1987) Other suitable liposomes include immunoliposomes (e.g. U.S. Pat. No. 4,957,735).

RNA preparations may also be administered to an organism with cells, such as stem cells. Administration may be simultaneous, separate or sequential. Cells and RNA of the invention may also be administered in simultaneous, separate or sequential application with other therapies effective in treating a particular disease. In one embodiment, RNA extractable from one or more stem cell types or stem cell active tissue(s) may be administered in simultaneous, separate or sequential application with cells, such as stem cells. For example, in preferred embodiments, whole embryo RNA, foetal RNA, neonatal RNA or juvenile RNA is administered in simultaneous, separate or sequential application with stem cells, particularly bone marrow stem cells. It is shown here that stem cell mediated tissue repair and regeneration is improved by co-injecting embryo-derived RNA fractions with stem cells.

In embodiments where RNA is administered to a subject in vivo, the RNA is preferably administered by one or more of the following methods:

### i) Systemic RNA Application

The RNA of the invention may be provided by systemic application. For example, the RNA may be provided by intra-venous, intra-arterial, oral, intra-osseous or sub-cutaneous injection or infusion.

### ii) Localised RNA Application

The RNA of the invention may be provided to a restricted region in the body of the recipient. For example, application may be localised to an organ or limb. Generally, the restricted region will comprise the target tissue to be treated.

In one embodiment, the region to which the RNA is provided may be defined by the circulation to that particular area of the body. For example, the RNA is provided by localised perfusion, or by infusion in one or more arteries supplying that particular area.

In another embodiment, the region may be defined by a distinct fluid region, such as the pleura, the peritoneum, the spinal cerebrospinal fluid or the ventricular cerebrospinal fluid. For example, the RNA is provided by localised injection or infusion into the fluid region of interest.

iii) Targeted RNA Application

The RNA of the invention may be provided in a manner that allows it to be preferentially taken up by a subset of cells or types of tissue, for example by the target tissue. In these embodiments, the RNA may be modified such that it is preferentially taken up by the subset of cells or types of tissue. For example, the RNA may be packaged in liposomes that comprise specific ligands for a particular cell type and be injected systemically to provide targeted application to that cell type. Other examples of possible modifications for optimising effective uptake by a target tissue are discussed above.

In this embodiment, the RNA may be provided by either systemic or localised application, as described above.

### iv) Multimodal RNA Application

RNA may also be applied by a combination of two or more of the above methods. In such embodiments, each administration may involve the same or different RNAs of the invention.

### v) Enhanced Application

RNA application may also be enhanced by administering one or more treatments to the subject that enhance the effective uptake the RNA by the target tissue. These treatments may be systemic or localised, as discussed above. The treatments may also be given simultaneous, separate or sequential treatments in relation to the administration of the RNA. For example, in embodiments where RNA is administered by systemic intravenous injection, the recipient may be given an intravenous injection of an RNAse inhibitor either prior, simultaneously or after the RNA administration in order to reduce the rate of degradation of the RNA in the circulation. Alternatively, for example instead of RNAse in the above-noted example, the recipient may be given an effective amount of inactive RNA to sequester RNA-binding species and RNAase in the circulation.

### Provision of Cells

The invention provides cells obtained by the methods of the invention. The cells may be provided as frozen cells in a suitable receptacle. The cells may be provided in culture. Extracts of the cells are also provided such as whole cell extracts.

In embodiments of the invention involving the administration of stem cells to the subject, the stem cells are preferably administered by one or more of the following methods:

### i) Systemic Stem Cell Application

The stem cells may be provided by systemic application. For example, the stem cells may be provided by intra-venous, intra-arterial, oral, intra-osseous or sub-cutaneous injection or infusion.

### ii) Localised Stem Cell Application

The stem cells may be provided to a restricted region in the body of the recipient. For example, application may be localised to an organ or limb. Generally, the restricted region will comprise the target tissue to be treated.

In one embodiment, the region to which the stem cells are provided may be defined by the circulation to that particular area of the body. For example, the stem cells may be provided by localised perfusion, or by infusion in one or more arteries supplying that particular area.

In another embodiment, the region is defined by a distinct fluid region, such as the pleura, the peritoneum, the spinal cerebrospinal fluid or the ventricular cerebrospinal fluid. For example, the stem cells may be provided by localised injection or infusion into the fluid region of interest.

### iv) Multimodal Stem Cell Application

Stem cells may also be applied by a combination of the above methods. In such embodiments, each administration may involve the same or different stem cells, which may or may not have been treated with RNA of the invention.

### Pharmaceutical Compositions and Medicaments

In some embodiments, the medicaments of the present invention are pharmaceutical compositions. Accordingly, the invention provides pharmaceutical compositions comprising the various RNA molecules, stem cells, and/or differentiated cells of the invention. The RNA molecules, stem cells and differentiated cells may be formulated with standard pharmaceutically acceptable carriers and/or excipients as is routine in the pharmaceutical art. Techniques for formulating cells and nucleic acids may be employed as appropriate. The cells or RNA may be provided in physiological saline or water for injections. The exact nature of a formulation will depend upon several factors including the particular substance to be administered and the desired route of administration. Suitable types of formulation are fully described in Remington's Pharmaceutical Sciences, 19^{th} Edition, Mack Publishing Company, Eastern Pennsylvania, USA, the disclosure of which is included herein of its entirety by way of reference. RNA-based pharmaceuticals are known in the art. For example, ‘Ampligen’ (Hemispherx Pharma) is a medicament comprising double-stranded RNA molecules.

The composition of the invention will typically, in addition to the components mentioned above, comprise one or more ‘pharmaceutically acceptable carriers’, which include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, sugars, and lipid aggregates (such as oil droplets or liposomes). Such carriers are well known to those of ordinary skill in the art. Compositions may also contain diluents, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier. A thorough discussion of pharmaceutically acceptable excipients is available in Gennaro (2000).

Compositions of the invention will generally be in aqueous form (e.g. solutions or suspensions), but they may alternatively be in dried form (e.g. lyophilised). Liquid formulation allows the compositions to be administered direct from their packaged form, without the need for reconstitution in an aqueous medium, and are thus ideal for injection. Compositions may be presented in vials, or they may be presented in ready-filled syringes. The syringes may be supplied with or without needles. A syringe will include a single dose of the composition, whereas a vial may include a single dose or multiple doses.

Compositions of the invention may be packaged in unit dose form or in multiple dose form. For multiple dose forms, vials are preferred to pre-filled syringes. Effective dosage volumes can be routinely established, but a typical human dose for injection has a volume of 0.5 ml.

The pH of the composition for subject administration is preferably between 6 and 8, preferably about 7. Stable pH may be maintained by the inclusion of a buffer in the composition (e.g. a histidine or phosphate buffer). The composition will generally be sterile and/or pyrogen-free. Compositions may be isotonic with respect to humans. Compositions of the invention may include sodium salts (e.g. sodium chloride) to give tonicity.

Compositions of the invention may include an antimicrobial, particularly when packaged in multiple dose format. The various RNA preparations and compositions used to provide the RNA discussed herein to the target cell may also comprise agents to increase the stability of the RNA. For example, they may comprise RNase inhibitors or other agents that stabilise and/or protect the RNA from degradation. The RNA preparations may also have been treated to remove other kinds of molecules, for example protease or DNase treatment may have been used to remove protein and/or DNA. Thus the composition may be substantially free from DNA and/or protein.

Some pharmaceutical compositions of the invention include combinations of RNA extracted from cells or tissues according to any one of the embodiments described above, either alone or in combination with stem cells. The cells of the invention may be administered to a subject together with other active agents, such as one or more anti-inflammatory agent(s), anti-coagulant(s) and/or human serum albumin (preferably recombinant), typically in the same injection. The cells will generally be administered to a subject essentially in the form in which they exit culture. In some cases, however, the cells may be treated between production and administration. The cells may be preserved (e.g. cryopreserved) between production and administration. Cells may be present in a maintenance medium.

Specific combinations of particular interest include RNA extracted from brain tissue, neurone cells, cortical neurones and the like, with stem cells, for example bone marrow mesenchymal stem cells; spine RNA with stem cells, for example with bone marrow mesenchymal stem cells; foetal RNA with stem cells, for example with bone marrow mesenchymal stem cells; embryo-derived RNA, such as embryonic stem cell RNA with stem cells, for example with bone marrow mesenchymal stem cells. Examples of treatments would include: for Alzheimer's Disease treatment of bone marrow stem cells with foetal brain RNA; for treatment of Parkinson's Disease, bone marrow stem cells with RNA from a culture of dopaminergic neuronal cells obtained form a juvenile donor; for heart disease, bone marrow stem cells treated with RNA from a juvenile or adult cadaver; for diabetes CD34+ circulation stem cells treated with RNA from pancreatic islet cells form the cadaver of a normal adult. For multiple sclerosis, bone marrow stem cells treated with RNA derived from primary cultures of oligodendroglia. Such compositions are for simultaneous, separate or sequential administration to a subject suffering from a disease that is amenable to treatment according to the invention (although in each case treatment may also be effected by direct administration of only the RNA to the recipient). Examples of such diseases are presented above. Where stem cells and RNA are to be administered together, they may be packaged separately or in admixture, and they may then be administered separately or in admixture.

A therapeutically effective amount of the medicament, compositions, cells or RNA molecules should be administered to a subject. The dose may be determined according to various parameters, especially according to the substance used; the species, age, weight and condition, including immuno-status, of the subject to be treated; the route of administration; and the required regimen. A physician will be able to determine the required route of administration and dosage for any particular subject. The dose may be determined taking into account the age, weight and conditions of the subject to be treated, the type and severity of the degeneration and the frequency and route of administration.

The amount of RNA in a composition of the invention will be sufficient to bring about the necessary desired alteration in a cell property. For example the concentration of RNA may be from 10 ng to 5 mg/ml, preferably from 100 ng/ml to 2.5 mg/ml, more preferably from 1 μg/ml to 500 μg/ml, even more preferably from 5 μg/ml to 100 μg/ml and still more preferably from 10 to 50 μg/ml. In a particularly preferred case the RNA concentration may be from 15 to 40 μg/ml, preferably from 20 to 35 μg/ml and in particular may be 25 μg/ml. In some cases, a total of 100 ng to 0.1 g, preferably from 1 μg to 50 mg, more preferably from 100 μg to 10 mg, still more preferably from 250 μg to 1 mg of RNA may be present. Any suitable concentration and/or amount of RNA may be present. A wide range of concentrations and/or amounts of RNA may be employed and the precise concentration and/or amount may be varied according to the method of delivery of the RNA to the target cells or tissues, the source of the RNA and whether the RNA is provided in vitro or in vivo.

The invention provides a pharmaceutical composition comprising an RNA of the invention (including RNA mimics, analogs, and modified RNAs), wherein the composition: (i) has a pH between 6 and 8; (ii) includes a buffer; (iii) is sterile; and (iv) is substantially pyrogen-free. The RNA in the composition is preferably homogenous. The RNA is preferably the active pharmacological agent within the composition. The composition is preferably located within a container that is labelled to indicate the composition's pharmaceutical purpose. The composition is preferably contained in an RNAse free container. The composition is preferably contained in a coloured bottle. The composition is preferably produced in an RNase free environment. The composition is preferably produced using reagents and chemicals that are essentially RNase free. The composition may comprise an RNase inhibitor.

### Applied Methods for Treating Subjects

The present invention provides numerous applications of the treatments discussed in “MEDICAMENTS AND METHODS FOR TREATING SUBJECTS” above.

### i) Pre-Treatment Ablation/Atrophy

Optionally, any of treatments a)-d) may be combined with ablation or atrophy of the target tissue before carrying out said treatment. Such ablation or atrophy is carried out, for example, to remove existing tissue that is in a compromised state, to provide space for new tissue growth, to provide additional signals to induce new tissue growth and/or to provide additional signals to indicate the desired localisation of new tissue growth.

### ii) Post-Treatment Growth Enhancement

Optionally, any of treatments a)-d) above may be combined with one or more conventional therapies for promoting growth in the target tissue. This will enhance a treatment response wherein there is new tissue growth within the target tissue. Such conventional therapies may be applied during, or preferably after, the treatment of the invention. However, where multiple treatments are administered (as in the combinatorial therapy of treatment d)), it is specifically envisaged that the conventional therapy may be carried out between individual treatments a)-c).

For example, when the target tissue is muscle tissue, the effects of the treatments of the present invention may be enhanced by exercise of said muscle tissue after treatments a)-d) above.

iii) Combined Treatment Schedules

In some embodiments of the present invention, the subject will be treated with one of treatments a)-d) defined above, delivered as a single treatment focused on one particular target tissue.

However, in other embodiments, the subject will be treated with multiple treatments a)-d), delivered as a series of treatments in a combine treatment schedule. Each one of these treatments may focus on a different target tissue. Preferably, the sequence of treatments in a combined treatment schedule will be selected so as to achieve a synergistic effect in the subject, for example by progressively repairing tissues that make up a specific system, e.g. a organ, within in the subject's body.

### iv) Combination Treatments

In some embodiments of the present invention, treatment with one of treatments a)-d) defined above will be the sole form of treatment of the subject.

However, in other embodiments, treatment will be combined with other forms of treatment as a “combination treatment”. The other forms of treatment include treatments known in the art for rectifying disease, damage or age-related loss-of-function in the tissue of interest. Alternatively, they may be generic therapies, like surgery or chemotherapy, that result in tissue damage, such that the treatments of the present invention may be used to repair this damage. In these embodiments, the tissues in which damage has been caused constitute the “target tissue” of the methods and medicaments of the invention. The source tissue is chosen accordingly, as discussed above.

For example, in a preferred embodiment, the other form of treatment is surgery, and the treatments of the present invention are used peri-operatively, as an adjunct to this therapy. For example, treatments of the present invention may be used post-operatively or pre-operatively, as discussed below.

Postoperative Combination Treatments

In this embodiment of the present invention, the subject undergoes a conventional form of surgery. Following surgery, the subject is provided with a treatment of the present invention. The target tissue is defined on the basis of the effects of the surgery. Accordingly, various specific embodiments of this invention are envisaged, depending on the nature of the surgery and the adjunctive benefits desired. These embodiments may be applied alone or in combination.

1) Treatment to Repair Tissues Damaged by Surgery.

In this embodiment, the target tissue comprises tissues that have been, or are expected to have been, damaged during the surgery. For example, the target tissue may comprise skin, muscle or blood vessels. These tissues may have been damaged in the surgery, for example during incision. In another embodiment, the target tissue may comprise parts of organs that may have been damaged. This embodiment will allow, for example, repair along rejoined tissue margins.

2) Treatment to Increase Organs or Tissue Function After Surgery.

In some embodiments, the surgery will involve the removal of part of an organ (e.g. the liver) or of a whole organ where more than one is normally present (e.g. a kidney). Accordingly, it will be desired for the function of the remaining organ tissue to be increased. In such embodiments, the target tissue may comprise the organ tissue involved.

3) Treatments to Rectify Systemic Damage Caused by Surgery.

Some operations, particularly those requiring general anaesthetics or carried out on elderly subjects, place systemic stress on the subject. Accordingly, it will be desired for the function of the tissues of the subject in general to be increased. In such embodiments, the target tissue may comprise all or most of the tissues of the body. Accordingly, the source tissue preferably comprises the tissues of a whole-organism or near-whole-organism. Moreover, it is preferred that the source tissue comprises tissues of a donor at an earlier developmental stage (e.g. foetal, neonatal or juvenile) than the subject of the treatment.

4) Treatments to Enhance the Function of Prostheses Added During Surgery.

Some operations involve the placement of one or more prostheses (e.g. replacement heart valves, vessel stents, replacement joints etc.) in a subject. Accordingly, it will be desired for the positive clinical effects of these prostheses to be enhanced. In such embodiments, at least two distinct target tissues may be defined. Each target tissue has a different clinical objective in mind.

The first is to effect regeneration at or around the attachment point(s) of the prostheses. According, the target tissue may comprise tissue that is found at such point(s).

The second objective is to increase function in any tissues that will experience a greater physiological load following surgery. Accordingly, the target tissue may comprise such tissues.

5) Treatments to Enhance the Function of Transplants Added During Surgery.

Some operations involve the transplant of an organ (e.g. a kidney or heart), part of an organ (e.g. liver lobe) or a specific tissue (e.g. skin or blood) in a subject. Accordingly, it will be desired for the positive clinical effects of the transplant to be enhanced. In such embodiments, at least four distinct target tissues may be defined. Each target tissue has a different objective in mind.

The first is to effect regeneration at or around the attachment point(s) of the transplant. According, the target tissue may comprise that is found at such point(s). In particular, the target tissue may comprise connective tissue and/or blood vessels that serve the transplant.

The second objective is to increase function of the transplant itself. Accordingly, the target tissue may comprise the tissues of the transplant. For example, the target tissue may comprise all of the tissues and cell types of the transplant (e.g. the target tissue may be whole kidney for a kidney transplant; or whole liver lobe for a liver lobe transplant; or whole heart for a heart transplant etc.). Alternatively, in another example, the target tissue may comprise only certain tissues or cell types of the transplant (e.g. the target tissue may be tissue of the sino-atrial node for a heart transplant). In such embodiments, the target tissue will generally comprise tissues that are known to be particularly at risk of early post-transplant degeneration or failure, such as the sino-atrial node in a heart transplant.

The third objective is to increase the function of other tissue in the subject that is at risk of early post-transplant degeneration or failure. Accordingly, the target tissue will comprise these tissues.

The fourth objective is to repair tissues that cannot be, or cannot completely be, surgically rejoined. Accordingly, the target tissue will comprise these tissues. For example, where nerve tissue and/or microvasulature tissue in the subject need to be joined with such tissues in the transplant, target tissue will comprise these tissues.

5) Treatments for Traumatic Resection During Surgery.

In some operations, tissue such as the whole of a damaged part of the body (e.g. an organ, such as a spleen or placenta; or a limb, such as an arm or leg) or part of a damaged part of the body (e.g. a part of an organ, such as a lobe of a liver; or a part of a limb, such as a finger) is deemed surgically irreparable and is resected for the protection of the subject, for example to prevent excessive loss of blood. In such operations, it will be desired for the function of the remaining organ tissue to be increased or for the removed tissue to be regenerated. In such embodiments, the target tissue may comprise the organ tissue involved. Preferably, the autologous resected tissue is itself used as the source of the source tissue for this embodiment. In a particularly preferred embodiment, an amputated limb may be used as source tissue to promote regeneration of said limb post amputation. In another preferred embodiment, a surgically removed cortex/hippocampus may be used as source tissue to treat brain injury.

Preoperative Combination Methods

Any of the above post-operative treatments may also be used pre-operatively, either instead of, or in addition to, the post-operative treatment. In such embodiments, the aim of the treatment may be to achieve the post-operative effects mentioned above without the need for a post-operative treatment. Alternatively, the aim of the treatment may be to enhance the post-operative effects mentioned above when a post-operative treatment is used in addition to the pre-operative treatment.

For example, where greater function will be required post-operatively in an organ, or where increased tissue strength or mass may be desired for the attachment or accommodation of a prosthesis, tissue enhancement according to methods described above, with appropriate definition of the target tissue and selection of the source tissue as discussed, may be performed pre-operatively (as well as post-operatively, if required).

### Engraftment of Genetic Modifications

The present invention provides a means for grafting genetically modified cells into tissue of a subject.

i) Engraftment of Tissue from Genetically Modified Bone Marrow.

In one embodiment of this aspect of the invention, the invention provides a method for grafting genetically modified cells into a target tissue of a subject comprising the steps of:

i) administering to the subject genetically modified stem cells to repopulate the bone marrow of the subject; and

ii) administering to the subject isolated RNA comprising an RNA sequence extractable from a source tissue such that engraftment of the cells within a target tissue is effected. The RNA may be extractable or extracted from the source tissue.

Accordingly, the present invention also provides the use of isolated RNA comprising an RNA sequence extractable from a source tissue in the manufacture of a medicament for grafting genetically modified cells into a target tissue of a subject, wherein the bone marrow of said subject has been repopulated with genetically modified stem cells.

The term “target tissue” as used in connection with this aspect of the invention means one or more tissues in the subject in which it is desired to graft genetically modified cells. A detailed description of potential target tissues is presented above and this aspect of the invention is equally applicable to the aspects set out in this description. In a preferred embodiment, the target tissue is muscle, particularly diaphragmatic muscle, (for example in the treatment of Duchenne muscular dystrophy). In another preferred embodiment, the target tissue is the lung (for example in the treatment of cystic fibrosis). In another preferred embodiment, the target tissue is the liver (for example in the treatment of haemophilia).

The stem cells may be any of the stem cell types described infra. However, it is preferred that the stem cells are capable of repopulating the bone marrow of the recipient. For example, the stem cells are preferably bone marrow mesenchymal stem cells. In a preferred embodiment, the stem cells are derived from the subject. For example, a sample of bone marrow may be taken from the intended recipient and used to provide a culture of stem cells. However, in another preferred embodiment, the stem cells are derived from a syngeneic source.

The stem cells are treated to generate a genetic modification in at least a sub-population of the cells. For example, a culture of stem cells may be treated in a manner known in the art to achieve a genetic modification. One such method is small fragment homologous replacement (see, for example, Sangiuolo et al (2002) BMC Medical Genetics 3, 8; Gruenert (1998) Curr Res Molec Ther 1, 607-613; Gruenert (1999) Gene Ther 6, 1347-1348; Kmiec (1999) Gene Ther 6, 1-3; Lai et al (1999) Exp Nephr 7, 11-14 and Woolf (1998) Nat Biotechnol 16, 341-344). Where the process of genetic modification does not achieve 100% efficiency, the sub-population of successfully modified cells may be selected and used to create a culture of genetically modified stem cells. Successful modification may be verified by any suitable method known in the art, for example by PCR of the relevant DNA sequence in the cells. Clones of genetically modified cells may be pooled. Alternatively, they may be used individually. Most preferably, a monoclonal culture of genetically modified stem cells is used. In a preferred embodiment, the genetic modification is a correction to wildtype sequence of a mutation in the dystrophin gene (for example, in the treatment of Duchenne muscular dystrophy). In another preferred embodiment, the genetic modification is a correction to wildtype sequence of a mutation in the cystic fibrosis transmembrane conductance regulator gene (for example in the treatment of cystic fibrosis). In another preferred embodiment, the genetic modification is a correction to wildtype sequence of a gene responsible for clotting factor (e.g. Factor VIII) production (for example in the treatment of haemophilia).

The genetically modified stem cells are used to provide a culture of stem cells suitable for repopulation of the bone marrow of the subject. Administration of the genetically modified stem cells to the subject results in the bone marrow of the recipient being repopulated with the stem cells. For example, the recipient may be subjected to a conventional process to repopulate its bone marrow with stem cells (as described in Hacein-Bey-Abina et al (2002) NEJM 346, 1185-1193).

Isolated RNA comprising an RNA sequence extractable from a source tissue is prepared as described infra.

Administration of the isolated RNA comprising an RNA sequence extractable from a source tissue to the subject may be achieved by any of the methods described infra, for example by intravenous injection. Preferably, the RNA is administered more than once, for example in a schedule of applications of 2, 3, 4, 5, 6, 7, 8, 9 or 10 applications.

Similarly, the source tissue is as described infra. In a preferred embodiment, the source tissue may comprise one or more tissues or one or more cell types from muscle tissue, and especially muscle from the diaphragm of a cadaveric donor, preferably a young cadaveric donor, (for example, in the treatment of Duchenne muscular dystrophy). In another preferred embodiment, the source tissue may comprise one or more tissues or one or more cell types from lung tissue (for example in the treatment of cystic fibrosis). In another preferred embodiment, the source tissue may comprise one or more tissues or one or more cell types from liver tissue (for example in the treatment of haemophilia).

In an example of this embodiment, Duchenne muscular dystrophy (DMD) may be treated in a human patient. In many cases of DMD, the sufferer carries a mutation at a single base position in the dystrophin gene. This results in aberrant muscle tissue. Accordingly, the engraftment of genetically modified cells that can give rise to functional muscle tissues provides an effective treatment of this disease. Preferably, after administration of the RNA, for example during a schedule of multiple applications, the patient undertakes exercises, e.g. stressed breathing exercises, to additionally stimulate growth and repair of, e.g. diaphragmatic muscle. In this example, growth and repair of muscle, particularly diaphragmatic muscle, is induced. This is achieved by the RNA treatment per se (as in the treatments described infra) and, where carried out, the exercise regime. A proportion of the muscle growth and repair is provided by progeny cells of the administered stem cells. Accordingly, such cells include the genetic modification. Preferably, the new muscle cells create entirely new muscle fibres. Also preferably, the new muscle cells provide additional myoblasts which fuse with pre-existing fibres and provide a supply of wild-type dystrophin to the whole muscle.

ii) Engraftment of Tissue from Ex Vivo-Cultured Genetically Modified Stem Cells.

In another embodiment of this aspect of the invention, the invention provides a method for grafting genetically modified cells into a target tissue of a subject comprising the step of:
i) administering to the subject genetically modified stem cells such that engraftment of the cells within a target tissue is effected;
   wherein said stem cells have been treated with isolated RNA comprising an RNA sequence extractable from a source tissue. The RNA may be extractable or extracted from the source tissue.

Accordingly, the present invention also provides the use of genetically modified stem cells that have been treated with isolated RNA comprising an RNA sequence extractable from a source tissue in the manufacture of a medicament for grafting genetically modified cells into a target tissue of a subject.

As discussed above, the term “target tissue” as used in connection with this aspect of the invention means one or more tissues in the subject in which it is desired to graft genetically modified cells. Preferred target tissues for this embodiment are described in relation to embodiment i) of this aspect of the invention above. The stem cells may be any of the stem cell types described infra. For example, the stem cells are preferably bone marrow mesenchymal stem cells. In a preferred embodiment, the stem cells are derived from the subject. For example, a sample of bone marrow is taken from the intended recipient and used to provide a culture of stem cells. However, in another preferred embodiment, the stem cells are derived from a syngeneic source.

The stem cells are treated to generate a genetic modification as described in embodiment i) of this aspect of the invention above. Preferred genetic modifications for this embodiment are again described in relation to embodiment i) of this aspect of the invention above.

Treatment of the stem cells with the isolated RNA comprising an RNA sequence extractable from a source tissue to the subject is achieved by any of the methods described infra.

Similarly, the source tissue is as described infra. Preferred source tissues for this embodiment are described in relation to embodiment i) of this aspect of the invention above.

Isolated RNA comprising an RNA sequence extractable from a source tissue is prepared as described infra.

Administration of the genetically modified RNA-treated stem cells to the subject is achieved by any of the methods described infra, for example by intravenous injection. Preferably, the stem cells are administered more than once, for example in a schedule of applications of 2, 3, 4, 5, 6, 7, 8, 9 or 10 applications.

Optionally, the method further comprises administering to the subject isolated RNA comprising an RNA sequence extractable from a source tissue, as described in embodiment i) of this aspect of the invention above.

Further optionally, the method may comprise administering genetically modified stem cells to repopulate the bone marrow of the subject, as described in embodiment i) of this aspect of the invention above.

In an example of this embodiment, Duchenne muscular dystrophy (DMD) may be treated in a human patient. Again, it is preferred that after administration of the RNA-treated stem cells, for example during a schedule of multiple applications, the patient undertakes exercises, as described in relation to embodiment i) of this aspect of the invention above.

iii) Further Modifications of the Embodiments i) and ii) Above.

The present invention provides various further modifications of embodiments i) and ii) to further enhance the engraftment of genetically modified cells in the target tissue.

For example, in a preferred embodiment, before treatment with RNA in embodiment i) or RNA-treated stem cells in embodiment ii), the target tissue is prepared to enhance engraftment. For example, the tissue may be partially or completely ablated or surgical resection. Ablation may be carried out by any suitable method known in the art, including radiation, chemical lesion, laser, treatment with depleting monoclonal antibodies targeted to cells with specific surface markers in the tissue, photo-ablation, sonic ablation and radiofrequency ablation. Such ablations may be conducted in a manner which minimises build-up of scar tissue. In embodiments where the target tissue is skeletal muscle, the muscle may be rested to induce a degree of atrophy. In some embodiment, the target tissue may be required to maintain a minimum level of function during treatment, so ablation may be applied selectively to part of the tissue involved. For example, ablation may be applied selectively to part of an organ, e. g. a lobe of a lung. In another example, ablation may be applied in a distributed fashion, e. g. as a pattern of ablation applied to the diaphragm.

In another preferred embodiment, after treatment with RNA in embodiment i) or RNA-treated stem cells in embodiment ii), the recipient is subjected to a regime that encourages tissue growth. For example, if the target tissue is skeletal muscle, the recipient may undertake a regime of exercise aimed at causing growth of the target muscle.

In both embodiments, the recipient may undergo a single treatment, or treatment may be repeated to maintain or enhance the degree of engraftment of genetically modified cells in target tissue. Furthermore, the recipient may undergo a number of different treatment, using difference source tissues, to effect engraftment in various target tissues. These treatments may be carried out in a defined sequence to allow sequential engraftment, for example within a specific organ.

In another preferred embodiment, the stem cells may have been genetically modified to increase expression of genes involved in tissue de-differentiation or regeneration prior to administration to the subject. In a specific embodiment, such modification may be the Msx1 gene. In another specific embodiment, the genetic modification to the stem cell may be over expression of one or more of the Insulin-Like Growth Factor (IGF) gene(s). IGF is well established as a key molecule in facilitating tissue regeneration (Barton-Davis, E et al (1998) PNAS 95. 15603.). In other preferred embodiments stem cells may be genetically modified to secrete molecules conducive to changing the stem cell niche or environment post transplantation e.g to secrete molecules that facilitate the integration and differentiation of the stem cells subsequent to implantation and thus enhance tissue regeneration or repair.

### In Vitro Methods for Inducing the Differentiation of Stem Cells.

In some aspects and embodiments of the present invention, stem cells that have been differentiated in vitro are used. The present invention therefore also provides methods for inducing the differentiation of stem cells in vitro. The differentiation is achieved by providing the cell with an RNA sequence comprising an RNA extractable from the cell type that it is desired to differentiate the stem cell into. The RNA may be extractable or extracted from cells comprising said desired cell type(s). In particular the invention provides a method of inducing in vitro totipotent, pluripotent or unipotent stem cells of a stem cell line or obtained from a tissue to differentiate into one or more desired cell types, which comprises providing isolated RNA comprising an RNA sequence extractable from tissue or cells comprising said desired cell type(s) to a cell culture of said stem cells under conditions whereby the desired differentiation of said stem cells is achieved.

Any stem cell may be used in the methods, including any of those mentioned herein. The stem cells may originate from the intended recipient of the methods or medicaments of the invention. In some cases the stem cells may originate from a recipient who has a genetic defect and preferably the genetic defect may have been corrected or ameliorated in the stem cells in such cases.

The RNA may be provided to the target stem cells using any of the methods discussed herein.

The stem cells may be induced into any desired cell type including any of those mentioned herein. In a preferred case the stem cell will be differentiated into a stable terminal differentiated cell type. A terminal differentiated cell type may generally be considered as one that does not naturally differentiate to give any other cell type and is typically at the end of a lineage. In some cases the stem cell may be differentiated into an intermediate cell between the stem cell and the terminal cell of the lineage. Such intermediates may have some degree of proliferative capacity.

The differentiated cell may be one of an organ or tissue such as the liver, spleen, heart, kidney, skin, gastrointestinal tract, eye, or reproductive organ. In a preferred embodiment the differentiated cell type may be one that is missing, present in reduced number or defective in a particular condition. The condition may be any of those mentioned herein and include injury, degenerative disease or a condition resulting from a genetic disorder. In a particularly preferred embodiment the differentiated cell may be an islet of Langerhans cell as the resulting cells can be used to treat diabetes. In another case the differentiated cell may be one of the central nervous system that can be used to treat a disorder or injury of the nervous system and particularly a disease of the brain or a spinal cord injury. In a preferred embodiment bone marrow stromal cells may be differentiated into neuronal cells.

In some cases the stem cell that is differentiated may be a pluripotent, but not totipotent, stem cell. In such cases the stem cell may, for example, be differentiated into a cell type that the stem cell is known to differentiate into in the organism it is isolated from.

In a preferred embodiment, bone marrow stromal stem cells may be differentiated into neuronal cells. In particular, they may be differentiated into neuronal cells expressing neuronal marker proteins (NeuN). Typically, the bone marrow stem cells may be differentiated into neuronal cells by providing an isolated RNA comprising RNA extractable from one or more types of brain cells or brain cell lines. In some cases the RNA may comprise an RNA extractable from brain tissue and in particular it may comprise an RNA extracted from a brain tissue. In a particularly preferred case the RNA may comprise RNA extractable from cortical neurones or a cortical neurone cell line. In some cases RNA extractable from neurones found in other locations than the brain may be employed or from cell lines derived from such neurones.

In another preferred embodiment, bone marrow stem cells may be induced to differentiate into muscle cells and in particular into skeletal muscle cells. Typically the RNA sequence provided will comprise an RNA extractable from or extracted from muscle cells or muscle cell lines and in particular from muscle stem cells.

The invention provides cells obtained using the above methods. The cells may be provided in some cases as frozen aliquots in suitable receptacles. The invention also provides cell extracts of the cells.

In some cases the stem cells may be present in or on a structure such as a support, membrane, implant, stent or matrix when they are differentiated or alternatively the differentiated cells may be added to such a structure. The structure may then be used in the manufacture of a medicament for treating any of the conditions mentioned herein. Mixtures of different differentiated cell types may also be made, for example, to mimic populations occurring together in vivo.

In one preferred embodiment the in vitro method may comprise:
providing a stem cell population and culturing it in vitro according to established protocols;
providing RNA extracted from a desired target tissue type (for example neurones, glia, muscle or any of the differentiated cell types mentioned above) to the stem cells; and
maintaining the cells in culture.

In a further preferred embodiment the in vitro method may additionally comprise the step of
extracting RNA from a desired target tissue type (for example neurones, glia muscle or any of the differentiated cell types mentioned above).
   In these embodiments of the invention, the RNA may be preferably be provided to the stem cells either 1) as naked RNA extract 2) via liposome mediated transfer 3) by electroporation of recipient cells or other established methods.

Preferably the resulting differentiated cells may then be formulated into a medicament which can be administered to a subject by an appropriate route such as via the sub-cutaneous, sub-dermal, intra-venous or intra peritoneal routes.

### General

The term “comprising” encompasses “including” as well as “consisting” e.g. a composition “comprising” X may consist exclusively of X or may include something additional e.g. X+Y.

The word “substantially” does not exclude “completely” e.g. a composition that is “substantially free” from Y may be completely free from Y. Where necessary, the word “substantially” may be omitted from the definition of the invention.

The term “about” in relation to a numerical value x means, for example, x±10%.

### MODES FOR CARRYING OUT THE INVENTION

The following Examples and Preparative Examples illustrate the invention. Although these include Examples are illustrative of the effects of the present invention in rodents, and therefore do not fall within the scope of the appended claims, the skilled person in the art would readily recognise their application to the treatment of human subjects.

### EXAMPLE 1

### Production of Neural and Muscle Cells from Bone Marrow Stromal Stem Cells

### Marrow Harvest and Culture.

Bone marrow stromal (mesenchymal) stem cells were obtained from adult Sprague Dawley rats. The technique is based upon the protocol of Owen and Friedenstein (1988), and represents a typical established adult stem cell source suitable for expansion in vitro. Briefly, after schedule one killing (cervical dislocation), tibia and femora were excised within 5 minutes of death. All connective and muscular tissue was removed from the bones and all further procedures were conducted under sterile conditions.

Marrow was expelled from the bones by flushing the bones with media (α-MEMS—Gibco Invitrogen Co. UK) containing 10% foetal calf serum, and 1% penicillin/streptomycin. Flushing was achieved by inserting a 25-guage needle attached to a 5 ml plastic barrel into the neck of the bone (cut at both distal and proximal end) and expelling 2 ml of media through the bone. The media and bone marrow sample were collected in sterile universal containers. Bone marrow cells were subsequently dissociated by gentle trituration through a 19-guage needle approximately 10 times. One ml of aspirate was then placed in six well plates (SLS Ltd. UK). Two ml of fresh α-MEMS was then added to each well giving a plating density of approximately 12,000-15,000 cells per ml. Plates were then incubated at 37° C., in 5% CO₂ in air and left undisturbed for 24 to 48 hours (Harrison & Rae, 1997).

Following this time period, marrow derived stem cells were isolated from non-plastic adherent cells by aspirating the culture media from the plate. Plastic adherent marrow stromal stem cells remained, and were supported by the addition of 2 ml of fresh α-MEMS (10% foetal calf serum and 1% penicillin/streptomycin). New media was applied every 48 hours until the plate was confluent with colony forming units (CFU's) confirmed by microscope analysis (Owen & Friedenstein, 1988, supra). Under optimal conditions this required 5 to 7 days at 37° C. Resultant cells were confirmed as stromal stem cells morphologically and immunohistochemically.

### RNA Procedure

Brain homogenate was prepared and RNA separated using an RNA commercial separation kit or standard phenol based procedures. In the initial experiment, RNA was prepared by a cold phenol extraction method based on the method of Kirby (1956). Brains were freshly dissected from eight freshly killed rats. Eight grams of brain, excluding the cerebellum, was weighed and 5 ml of phosphate buffered saline (PBS) was added. The mixture was homogenised in a glass Teflon homogeniser for approximately 4 minutes. An equal volume of 95% saturated phenol was added. The resultant solution was left at room temperature for 15 minutes then centrifuged at 18,000 rpm in an ultra centrifuge for 30 minutes. The aqueous phase was retained and brought to a concentration of 0.1M MgCl₂ by the addition of 1M MgCl₂. Two volumes of ethanol were then added and precipitation was allowed to occur for approximately 30 minutes. A final spin at 6,000 rpm for 15 minutes produced an RNA rich precipitate, which was retained and stored under ethanol. Resultant RNA was air dried and dissolved in 6 ml of fresh media as defined above.

One ml of media containing the RNA was added to each well of confluent bone marrow stem cells for 24 hours. After 24 hours the RNA media was removed and replaced with fresh media. Cells were observed for phenotypic change every 12 hours.

Further, cells were subjected to immunohistochemical analysis to confirm that the RNA induced in the bone marrow stem cells was a neuronal phenotype. This was achieved by testing treated cells for the expression of a neuronal marker NeuN. The results obtained are indicated in the Table below.

| Cells | Morphology | NeuN |
|---|---|---|
| Untreated cells | Retained CFU morphology | − |
| Brain RNA treated cells | Developed Neuronal type | + |
| | Morphology | |

Examination of the cells showed the RNA induced change in cellular differentiation to a clear neuronal phenotype 24 hours after application of brain derived RNA. Untreated bone marrow stem cells retained the classic colony forming unit morphology. However, as early as 12 hours post-treatment the brain RNA treated stem cells showed typical neuronal and glial morphologies. Further, these cells expressed a commonly used immunochemical marker for neurones. Control cells did not. This change in phenotype survived passage (×3) and thus would appear a stable change in recipient stem cell differentiation. That donor tissue RNA was responsible for the change in stem cell differentiation was confirmed by subsequent experimentation in which the inductive effect of RNA was abolished by pre-treatment with RNaze, yet remained resistant to treatment of the donor brain RNA with trypsin, a potent protease.

The experiment was repeated using donor RNA, derived from skeletal muscle to confirm the specificity of the induced differentiation. It was clearly visible that the stem cells prepared as above and treated with muscle derived RNA (prepared using a commercially available kit, RNAzol), showed a stable differentiation change to muscle phenotype. This was confirmed by immuno staining with Phospholamban and Phalloidin. In the muscle study, the stem cells were exposed to muscle derived RNA (derived with a different RNA separation technique) via a different method of RNA delivery. RNA was delivered to the stem cells via liposomes prepared after the methodology of Felgner et al. (1987). Thus it can be concluded from these studies that the induction in stem cells is specific to the donor tissue source, and that the RNA can be added to the stem cells via a variety of techniques commonly employed to deliver nucleic acids to cells.

### EXAMPLE 2

### The Effects of Brain RNA Differentiated Stem Cells on Age Related Damage to the Rat Brain, Assessed by Spatial Learning and Memory Performance of Recipient Animals

Bone marrow mesenchymal stem cells were prepared in vitro as described above in Example 1. When the cells reached confluence, they were exposed to brain RNA (prepared as above) for 12 hours. Donor stem cells were derived from a pigmented rat strain (Lister Hooded). Donor RNA and recipient animals were provided from a different rat strain (Sprague Dawley).

Recipient Sprague Dawley rats were ex-breeder male rats aged between 468-506 days. It is well established that such animals of advanced age cannot learn to locate a hidden platform in a water maze (Stewart & Morris, 1993; Bagnall & Ray, 2000). Experimental animals received a 0.5 ml intra-venous injection of brain RNA treated stem cells, equating to the product of one six well plate of brain RNA treated cells. Control animals received an equivalent amount of untreated stem cells. Briefly, cells were collected from plates, either treated (experimental) or untreated (control) by mechanically removing them from the plastic plates using a rubber policeman and collected, by aspiration, in culture media. Cells were concentrated via a 5 minute spin at 1000 rpm and brought to a concentration outlined above. All injection procedures were conducted blind. For both groups, injections were mediated via the tail vein.

Fourteen days after injection, the aged rats were assessed blind on a commonly used spatial learning task, the Morris water maze. Each animal received 3 swims per day over a 3 day period with an inter trial interval of 10 minutes (Stewart & Morris, 1993). Latency to find the platform on each trial was recorded for each animal. Each trial consisted of a 60 second swim. If after that interval the animal had not located the platform, it was gently guided to the platform by the experimenter. Upon reaching the platform, the animal was allowed 10 seconds to orient to its location prior to removal to the home cage. Learning is evidenced by a decrease in time to locate the platform over repeated trials.

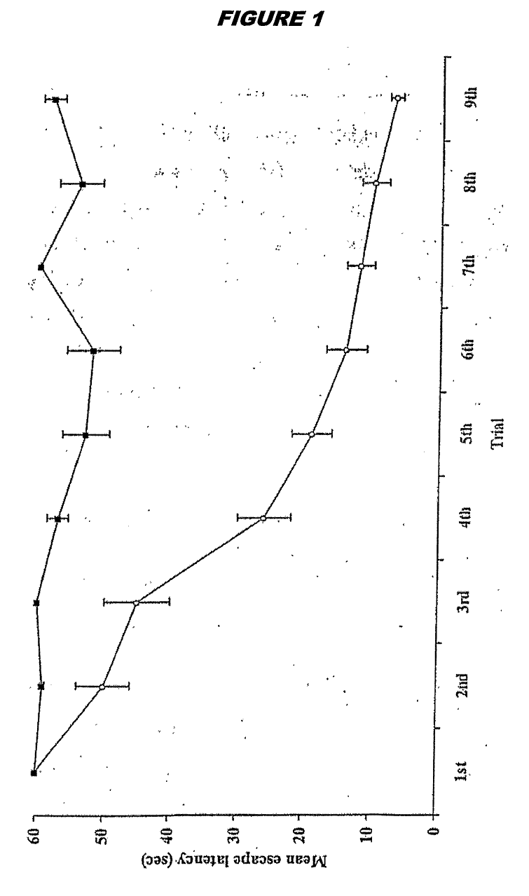

The results of the study are presented in FIG. 1. Control rats (n=9) receiving intravenous stem cells which had not been exposed to RNA, could not learn this task without a decrease in response latency over trials. However, the experimental animals receiving brain RNA treated stem cells showed a remarkable learning ability comparable to that of young rodents (p<0.0000000001). This study shows that RNA treated stem cells can significantly ameliorate age related deficits in spatial learning. Control untreated stem cells cannot.

The results confirm that RNA differentiated stem cells can repair age related damage by restoring behavioural capabilities.

### EXAMPLE 3

### In Vivo Stimulation of Resident Stem Cells Via Exogenous RNA Stimulated Differentiation, Migration and Integration

Given the powerful stimulatory effects of exogenous RNA on stem cells established in Examples 1 and 2, and the effects of these cells on repairing age related damage in a mammalian model, a further Example is given, establishing the effects of primary tissue derived RNA on host animal resident stem cells. To this end, neonate rats received an intraperitoneal injection of donor GFP-expressing crude bone marrow at age 1 day postnatal. Each animal received approximately 800,000 cells in a 0.2 ml injection. These foreign cells were readily integrated in host bone marrow and were observed to contribute to this biological environment. At age 90 days, GFP bone marrow grafted animals were randomly assigned to two groups.

Experimental animals received an injection of brain RNA, control animals received an injection of physiological saline. Experimental brain RNA was prepared as outlined in Example 1. Injection was conducted sub-cutaneously. Each animal received one whole brain equivalent of donor RNA in a 0.5 ml injection. Controls received an equivalent injection of physiological saline.

The results obtained showed a significant thickening of recipient cortex (p<0.0001) in experimental animals compared to control animals. Further, a significant number of differentiated neurones and glia in experimental animals showed expression of GFP indicating infiltration of resident bone marrow stem cells into the brain following application of exogenous brain RNA.

### EXAMPLE 4

### Induced Differentiation of Stem Cells Via Exogenous RNA Isolated from a Primary Cell Culture of Cortical Neurones

A purified culture of embryonic cortical neurones was established in the laboratory following the protocol of Saneto and deVellis (1987). Briefly, time mated Sprague Dawley female rats were sacrificed at day 16 of gestation. The abdominal area was sterilised with 70% alcohol and the uteri exposed. Uteri containing the embryos were then dissected free from the uteri and placed in a large 100 mm Petri dish. All the above procedures were conducted on a clean bench outside the sterile hood to prevent contamination. All further procedures were conducted under sterile conditions.

Intact uteri were then washed with physiological saline and transferred to another sterile Petri dish. Embryos were then dissected free from the uteri and placed in a new Petri dish for brain dissection. Brain tissue was exposed and gently removed with a spatula and cortices were dissected under a dissecting microscope. Meninges were then dissected clear in physiological saline. After cortices were processed, they were gently disrupted with repeated passage through a 10 ml glass pipette. The cell suspension was then passed through a Nitex 130 filter (mesh size 130 μm) and the filtrate centrifuged at 40 g. The pellet was then re-dispersed in serum free basal media (Saneto & deVellis, 1987, supra) and passed through Nitex 33 (mesh size 33 μm) and cells counted.

The suspension was supplemented with insulin (5 μg/ml) and transferrin (100 μg/ml) to form neurone-defined medium. Cells were seeded at a density of 1×10⁵ per well on 24 well culture plates pre-coated with polylysine (2.5 μg/ml). Cultures are reported as containing more than 95% neurones by immunological criteria of expressing the marker neurofilament protein, while not expressing the biochemical and immunological markers for astrocytes and oligodendrocytes (Saneto & deVellis, 1987, supra). Media was changed every third day post plating and cultures were maintained for 12 days prior to RNA extraction.

RNA was extracted from the primary cortical neurone cultures via a commercial kit (RNAzol) using the manufacturer's protocol. Resultant RNA was collected and redissolved in bone marrow culture medium (as defined in example 1) just prior to application to a confluent colony of rat bone marrow cells prepared as in Example 1. Each recipient bone marrow culture well received the total RNA extracted from one complete 24 well primary neuronal culture (although similar results were obtained a wide variety of exogenous RNA concentrations).

Bone marrow stem cells were examined microscopically 24 hours after application of exogenous RNA dissolved in media. Control bone marrow stem cells received an equal amount of RNAzol prepared bone marrow stem cell RNA.

Results showed all experimental stem cell wells produced clearly differentiated neurones, which stained positively for neuronal markers. No observable change in stem cell differentiation was found in the Bone marrow RNA treated wells. These results suggest that donor RNA from a purified cell source may induce highly specific stem cell differentiation.

The differentiation inducing effect of exogenous RNA fractions was sensitive to pre-treating the donor RNA with RNaze yet insensitive to trypsin. This suggests that RNA mediated the effect. These effects may be repeated using a wide range of RNA doses delivered exogenously by a variety of delivery methods and vehicles including liposomes or electroporation.

### EXAMPLE 5

### Specific Stem Cell Differentiation Induced by Exogenous RNA

### Marrow Harvest and Culture.

Bone marrow stromal (mesenchymal) stem cells were obtained from adult Sprague Dawley rats. The technique is based upon the protocol of Owen and Friedenstein (1988), and represents a typical established adult stem cell source suitable for expansion in vitro.

Briefly, after schedule one killing (cervical dislocation), tibia and femora were excised within 5 minutes of death. All connective and muscular tissue was removed from the bones and all further procedures were conducted under sterile conditions.

Marrow was expelled from the bones by flushing the bones with media (α-MEMS—Gibco Invitrogen Co. UK) containing 10% foetal calf serum, and 1% penicillin/streptomycin. Flushing was achieved by inserting a 25-guage needle attached to a 5 ml plastic barrel into the neck of the bone (cut at both distal and proximal end) and expelling 2 ml of media through the bone. The media and bone marrow sample were collected in sterile universal containers. Bone marrow cells were subsequently dissociated by gentle trituration through a 19-guage needle approximately 10 times. One ml of aspirate was then placed in six well plates (SLS Ltd. UK). Two ml of fresh α-MEMS was then added to each well giving a plating density of approximately 200,000 cells per ml. Plates were then incubated at 37° C., in 5% CO₂ in air and left undisturbed for 24 to 48 hours (Harrison & Rae, 1997).

Following this time period, marrow derived stem cells were isolated from non-plastic adherent cells by aspirating the culture media from the plate. Plastic adherent marrow stromal stem cells remained, and were supported by the addition of 2 ml of fresh α-MEMS (10% foetal calf serum and 1% penicillin/streptomycin). New media was applied every 72 hours until the plate was confluent with colony forming units (CFU's) confirmed by microscope analysis (Owen & Friedenstein, 1988, supra). Under optimal conditions this required 5 to 7 days at 37° C.

Positive control cells were primary cultures of embryonic whole brain (E18) maintained on identical 6 well plates

### Experimental Design

Plates of cells were randomly assigned to 5 groups of treatment:
Group 1: Brain RNA on BMSC 150 μg/ml
Group 2: Brain RNA+RNase 150 μg/ml
Group 3: Spleen RNA on BMSC 150 μg/ml
Group 4: No RNA added to BMSC
Group 5: Positive Control E18 brain primary culture

### RNA Methods

### RNA Extraction—Acid Guanidinium Thiocyanate-Phenol-Chloroform Method

RNA extraction was modified to further minimize DNA contamination by an additional step of DNase treatment (Ambion). Purity and concentration was confirmed by analysis on Nanodrop spectrophotometer.

Tissue Preparation.
1. Add tissue to 1 ml Solution D (@4° C.)
2. Homogenise

RNA extraction.
1. Add 0.1 ml sodium acetate (0.2M, pH 4.0)
2. Invert to mix
3. Add 1 ml of phenol chloroform-iso-amyl alcohol (25:24:1)
4. Shake vigorously for 10 seconds
5. Cool on ice for 15 min.
6. Transfer solution to 2 ml tubes—1.2 ml in each
7. Centrifuge at 10,000 g for 20 min @4° C.

RNA Precipitation.
1. Transfer the top (aqueous) phase to a fresh tube, max of 0.5 ml.
2. Add 1 ml isopropanol
3. Incubate at −20° C. for 5 min to precipitate RNA
4. Centrifuge at 10,000 g for 10 min @4° C. RNA pellet should be seen at base of tube.
5. Discard the supernatant, air dry the pellet. Do not allow the pellet to dry out completely as this will make the pellet very difficult to resuspend.
6. Dissolve the RNA pellet in 50 μl RNase free water for 10 min.
7. Transfer immediately to ice before use, or to storage at −20° C.
8. Repellet RNA and treat with DNase

DNase Protocol
Dilute each of the RNA samples in 1000 μl nuclease free water.
Put 100 μl into a well of a 96 well plate.
Add 10 μl DNase I buffer to each well.
Add 2 μl rDNase I.
Incubate at 37° C. for 30 minutes.
Add a further 2 μl rDNase I.
Incubate at 37° C. for 30 minutes.
Transfer two of each well to a 0.5 cm³ eppendorf.
Add 20 μl DNase Inactivation Reagent.
Incubate at room temperature for 2 minutes, vortexing a few times.
Centrifuge at 10,000 g for 90 seconds and transfer the RNA to a fresh tube.

Solution D

| | | | |
|---|---|---|---|
| 2-mercaptoethanol | 0.36 | ml | |
| Add 2-mercaptoethanol to Solution A | | | |
| Solution A | 50 | ml | Shelf life 1 month at |
| | | | RT |

Solution A

| | | | |
|---|---|---|---|
| Guanidinium thiocyanate | 250 | g | |
| Distilled water | 293 | ml | |
| Sodium citrate (0.75 M, pH 7) | 17.6 | ml | |
| Sarcosyl, 10%@65° C. | 26.4 | ml | Solution A shelf life |
| | | | 3 months at RT |

RNase Treatment

One sample of brain RNA was treated with RNase prior to addition to the cell sample in Group 2.
Remove the ethanol from the RNA sample and air dry for 10 minutes.
Add 0.25 cm³ of the RNase (at a concentration already made up of 1 mg/cm³ in PBS). Each RNase tube will give enough RNase for 4 eppendorfs of RNA.
Incubate the RNA+RNase at 37° C. for 5 hours.
Add 0.5 cm³ Bentonite (stock solution 10 g/cm³ in PBS).
Incubate for 1 hour at room temperature
Microcentrifuge for 10 minutes at 1500 rpm to precipitate the Bentonite.
Carefully remove the supernatant and use.

### Results & Conclusion

By day 4 post treatment there was clear morphological changes in the brain RNA treated stem cells. Cells could be clearly identified as neuronal and glial by their morphology. All RNA treated wells showed the same morphologies and distribution of these morphologies as the positive control wells (Group 5) primary cultures of brain tissue. RNase (Group 2) treatment destroyed this differentiation effect showing the active inducer of differentiation to be the RNA fraction. Specificity of the differentiation was confirmed by the spleen RNA treated group. Here, differentiation of the stem cells showed a different morphology involving aggregates of rounded cells with a spleenocyte-like morphology. Untreated BMSC retained their normal morphology throughout the experiment.

Cells were maintained in culture for 9 weeks and taken through 4 passages. Each group maintained their induced differentiation.

DNase-free naked RNA added to BMSC in culture can induce specific differentiation appropriate to the donor tissue. This transformation is stable over time and passage of the cells. The active inducer of the differentiation is RNA as the effect is destroyed by degradation of the RNA by RNase.

### EXAMPLE 6

### Induction of Nerve Tissue Specific Expression of Fluorescence in BMSCs by Exogenous RNA

Bone marrow stem cells were extracted from the tibias of B6.Cg-Tg(Thy-CFP) 23 Jrs/J mice (Jackson Laboratory USA) and maintained in culture using the methods outlined above. These mice express a special variant of GFP (cyan-CFP) at high levels in motor and sensory neurones, as well as a sub set of central neurones. No expression is detected in non-neural cells.

Cultures were maintained in 6 well culture plates for 18 days (approx. 80% confluent) with a media change every 72 hours. Cells were observed under fluorescence microscopy to confirm no expression of Neurone specific fluorescence in BMSCs.

RNA was extracted as reported in Example 5 from C57/black wild type mouse brain (adult) and analysed for purity and concentration as reported in Example 5.

RNA was added to BMSCs at 120 μg/ml in serum free media with an exposure time of 60 minutes. After exposure to RNA cells were washed with fresh media and maintained in long term culture.

Control BMSC were exposed to serum free media for one hour with no RNA and similarly maintained.

### Results

Seventy two hours after treatment with wild type C57/black mouse brain RNA, B6.Cg-Tg(Thy-CFP) 23 Jrs/J BMSCs showed some fluorescence. No fluorescence was evident in control (no brain RNA) B6.Cg-Tg(Thy-CFP) 23 Jrs/J BMSCs. Cells were observed at every media change and bright fluorescence was evident in all treated wells throughout the 4 month duration of the study. At no time interval did any untreated well show any fluorescence.

Further, Brain RNA treated BMSCs showed extensive morphological changes towards a neural phenotype.

### Conclusions

Expression of cyan-CFP at high levels in differentiated BMSCs confirms that the stem cells had been induced to differentiate into neural tissues. Further, this was a very stable differentiation as the RNA induced differentiation persisted for at least 4 months in culture.

The experiment was also repeated using the same protocols using a control group which received muscle RNA which should not induce Cg-Tg(Thy-CFP) 23 Jrs/J BMSCs to fluoresce. Experimental Cg-Tg(Thy-CFP) 23 Jrs/J BMSCs exposed to wild type brain RNA again showed extensive and persistent fluorescence. Muscle RNA induced cells showed no evidence of fluorescence and clear muscle like morphology.

Thus, this study confirmed the initial RNA induced specific differentiation of the stem cells and showed that neural differentiation was only induced by neural derived donor RNA.

### EXAMPLE 7

### Comparison of Spine RNA Treated Bone Marrow Stem Cells with Undifferentiated Bone Marrow Stem Cells in an Animal Model of Motor Neurone Disease

The SOD 1 mouse is a well-established animal model of human motor neurone disease. These transgenic animals begin to show hind limb paralysis at 70-90 days with aggressive loss of motor neurones and death at 120-135 days.

Thirty animals were used in the study. All were confirmed to express the SOD 1 genotype. Animals were randomly assigned into three groups as follows:
(i) group 1—bone marrow stem cells incubated with spine RNA;
(ii) group 2—bone marrow stem cells only; and
(iii) group 3—PBS injection.

Donor bone marrow stem cells were harvested and cultured as described in Example 1. Spine RNA was prepared from freshly dissected adult C57/B1 mice using the Kirby protocol described in Example 1. Stem cells to be used in group 1 were incubated with spine RNA for 5 hours (250 μg/ml), washed twice in fresh media, and then concentrated for injection at approximately 90,000 cells per animal in 0.1 ml. Stem cells prepared for injection in group 2 were maintained in culture with no exposure to RNA and given 5 hours equivalent exposure to fresh media.

Recipient animals in each group received an injection via the tail vein. Injections were mediated using a 30 G needle. Injections were performed on recipient animals between the ages of 72 and 86 days at which time all animals showed hind limb paralysis. The number of animals surviving in each condition was recorded daily. Further limb movement was assessed weekly on a simple run test to observe hind and forelimb function.

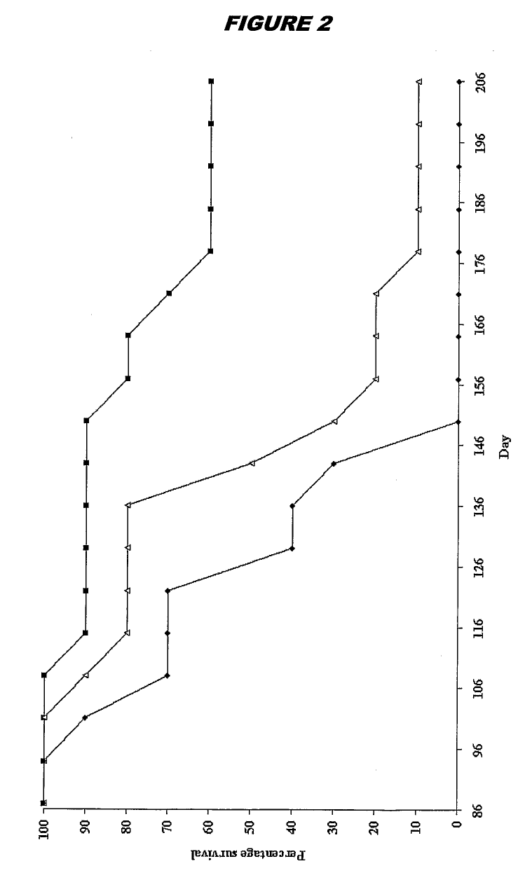

The results of this study are illustrated in FIG. 2. Pre-treatment of stem cells with spine derived RNA dramatically improved the efficacy of stem cell treatment in an established model of progressive neurodegenerative disease. Untreated bone marrow derived stem cells did have some effect but the novel step of pre-differentiating stem cells with RNA dramatically improves the effect. It is further noted from this example that all surviving animals in the RNA stem cell group (6) and the survivors in the stem cell only group (1) had complete recovery of pre-treatment paralysis and the treatment prevented further evolution of this normally progressive disease.

### EXAMPLE 8

### Effects of RNA Donor Tissue Age and Developmental Stage on Stem Cell Migration, Integration and Repair

Having established the effects of donor tissue derived RNA on stem cells in a variety of applications, a further example is provided investigating the effects of donor tissue developmental stage, prior to RNA extraction, on stem cell proliferation, migration and integration into host tissue.

Bone marrow stem cells were harvested and cultured as outlined in Example 1 from Tau-GFP-expressing mice. Recipient animals (N=24) were 254-299 day old C57/B1 mice randomly assigned to three recipient groups (n=8). Cultures of stem cells were randomly allocated to three conditions for RNA treatment prior to injection:
(i) group 1 foetal (E15) brain RNA+stem cells;
(ii) group 2 adult (90 day) brain RNA+stem cells; and
(iii) group 3 stem cells+no RNA. 1

RNA was extracted using the Kirby method as detailed in Example 1 and the appropriately sourced RNA detailed above was dissolved in media at a concentration of 200 μg/ml. Each well of recipient stem cells was incubated in 2 ml of fresh media supplement with 1 ml of RNA containing media (groups 1 & 2) or 3 ml of fresh media only (group 3) for 12 hours. Cells were then washed twice and concentrated for injection at approximately 40,000 cells in 0.3 μl of fresh media. Recipient animals were anaesthetised and cells were injected using stereotaxic guidance into the left lateral ventricle of the brain. Twenty days after surgery all groups were assessed on a mouse Morris water maze using the same training protocol as reported for rats reported in Example 2. Mice at this age show similar spatial learning deficits to old rats using this training methodology. After training, recipient rats were sacrificed and brain tissue was examined for cortical thickness and fluorescent microscopy to assess survival, proliferation and migration of GFP-expressing cells.

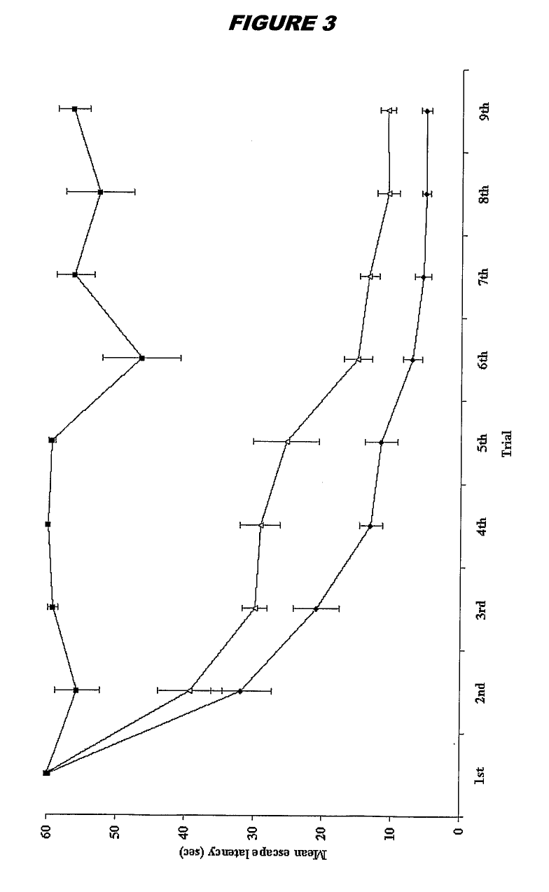

Behavioural results are presented in FIG. 3. Animals in both groups 1 and 2 showed excellent learning on the Morris water maze when compared to animals in group 3. This further shows the stimulatory effect of exogenous RNA treatment on stem cells in repairing age related brain damage (see Examples 2 and 5). Further, the foetal RNA+stem cell group showed significantly (p<1×10⁻¹⁰) faster acquisition of the task than the adult RNA+stem cell group. These data indicate that RNA sourced from a developmental stage when extensive neurogenesis is occurring may have a more profound effect when used to treat stem cells for tissue repair. Examining cortical thickness further supported this conclusion.

Measurement of cortex thickness in 20 identical anatomical cross sections in each animal showed a significant difference between the adult RNA+stem cells recipients and the stem cell only group (p<1×10⁻⁵), this confirms similar rat data (see Example 3). However, the cortex measures in the foetal RNA+stem cell group was also significantly thicker than the adult RNA group. Optical examination under fluorescent microscopy showed that the adult RNA+stem cell group had GFP-expressing cells extensively throughout the injected and contralateral hemispheres. However, foetal RNA+stem cell animals had approximately 30% more cells than the adult RNA group throughout the cortex of both hemispheres. GFP-expressing cells in the stem cell only group was predominantly located around the lower margins of the injected lateral ventricles and the olfactory bulbs. Only occasional cells were located in the ipsilateral cortex.

It can be concluded from this study that pre-treatment of stem cells with brain derived RNA increases their proliferation, migration and functional integration into recipient nervous systems. Further, RNA sourced from a more immature developmental stage, at an active cell generative stage, may have a more profound effect on stem cell stimulation and their consequent ameliorative effect in both age and disease related damage.

### EXAMPLE 9

### A Comparison of the Stimulatory Effects of Adult Stem Cell Derived RNA on Endogenous Neural Stem Cells and Their Activity

Evidence provided in Example 3 shows that exogenous RNA had a stimulatory effect on resident bone marrow stem cells in restoring age related behavioural deficits. This Example investigates if direct injection of bone marrow stem cell derived RNA can stimulate endogenous repair mechanisms to ameliorate age related behavioural deficits. Various endogenous neural repair processes are now known, including direct neurogenesis mediated by neural stem cells, but also secretion of survival factors from stem cells, which may influence damaged differentiated tissues. Bone marrow stem cells were harvested and cultured in vitro as described in Example 1. Confluent cultures were then selected for RNA extraction. RNA extraction was mediated using a commercial product RNAzol following the manufacturer's instructions. Resultant bone marrow RNA was dissolved in PBS (200 μg/15 μl) ready for injection into recipients.

Recipient Sprague Dawley rats were ex-breeder males aged between 433 days and 570 days. Due to profound age related damage to the CNS such animals cannot learn or recall the Morris water maze task. Recipients were matched for age into two groups of 10 animals:
(i) group 1—received a 15 μl injection of stem cell RNA; and
(ii) group 2—received a 15 μl injection of stem cell RNA treated with RNaze (see Example 1).

Injections were made under anaesthesia into the right lateral ventricle under stereotaxic guidance. Briefly, recipient rat was anaesthetized, head shaved and placed in a stereotaxic frame. Skin was swabbed with 100% alcohol and the skull exposed by longitudinal incision. A 1.5 mm wide hole was drilled 1.5 mm anterior to the bregma and 1.5 mm lateral to the midline. The visible dura was cut with the tip of a 30 G hypodermic needle. The loaded cannula was lowered into the lateral ventricle via stereotaxic guidance and the contents ejected in 5 μl steps. The cannula was left in place for 2 minutes before removal and the incision closed with suture.

Fourteen days after injection, the aged rats were assessed blind on the Morris water maze as described in Example 2.

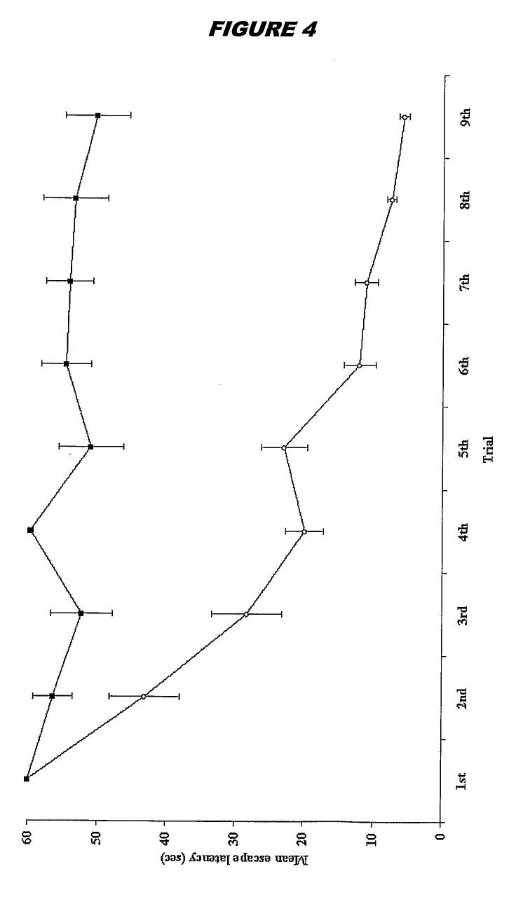

Results of this study are presented in FIG. 4. Control rats receiving deactivated stem cell RNA (RNaze treated) could not learn the task. There was no decrease in response latency over trials.

However, the stem cell RNA treated animals all learned the task and were comparable in performance to young rats.

The stem cell derived RNA had a significant (p=1.28×10⁻⁴⁵) effect on stimulating endogenous repair mechanisms in the aged recipient brain. This may have been mediated by stimulation of resident neural stem cell neurogenesis per se or by increased production of secretory molecular products involved in tissue repair.

This experiment has also been replicated with a similar stimulatory effect using foetal (E12) derived whole brain RNA injected at a dose of 125 μg/μl (n=8) and a PBS injected control (n=8). Foetal RNA injected animals performed significantly better than control (p<1×10⁻⁵). This replication indicates that RNA prepared from developmental stages known to show increased stem cell activity may also be used to stimulate endogenous repair mechanisms.

### EXAMPLE 10

### The Use of Rat Embryo RNA to Enhance Stem Cell Involvement in Tissue Regeneration

Adult mammals, including human beings, have poor regenerative abilities in many tissues and organs compared to foetal stages, which often have extensive regenerative abilities. Two major factors associated with this loss of regenerative ability are scar tissue formation and loss of secretory molecules that recruit new cells to injured tissues. While many laboratories have reported the integration of injected stem cells into damaged tissues, this has been on a relatively small scale. It could be hypothesised that if the signalling mechanisms of the foetal stage could be recapitulated in the adult, this would improve the ability of stem cells to effect major regeneration of structures which show little or no repair. This would include old established injuries with associated scaring which is known to inhibit stem cell migration, integration and repair potential. The methodology used is co-injection of whole embryo RNA with stem cells. The example provided illustrated the complete regeneration of an established ear punch hole lesion in adult rats following injection of whole rat embryo RNA and bone marrow stem cells.

15-day old fetuses were dissected from the uteri of time-mated Lister Hooded rats. Foetal tissues were disrupted mechanically by a Turex homogenizer in cold PBS. RNA was extracted using the Kirby protocol described in Example 1.

Bone marrow stem cells were cultured as described in Example 1 and concentrated for injection as described in Example 7.

The injury model involved 18 male Lister Hooded rats aged between 137 and 149 days at time of injection. All rats received a 1.5 mm hole punch injury to the left ear at 30 days prior to injection date to model an old established injury. Rats at this age do not regenerate ear tissue.

Experimental animals (n=6) received a tail vein injection of 800 μg of embryo RNA dissolved in 0.3 ml of PBS. One hour later, the animals received a second injection of approximately 2×10⁵ bone marrow stem cells suspended in 0.3 ml of a-MEMS culture media. Control animals (n=6) received an initial tail vein injection of approximately 2×10⁵ bone marrow stem cells followed by a second injection of 0.3 ml PBS 1 hour later. A further group, no treatment controls (n=6), were ear clipped but received no treatment.

Animals were observed daily for any signs of regeneration of ear injury. Results showed no evidence of tissue repair or remodelling in the no treatment control group. Similarly, the stem cell only injected controls also failed to show any evidence of repair other than a slight inflammatory response lasting 17 hours in one animal around the site of the injury. The experimental animals treated with a combination of embryo RNA and stem cells showed complete closure of the injury in all animals between 6 and 9 days post injection. In 5 of the 6 experimental animals there was complete remodelling of the injury to the extent that there was no visible scar or evidence of the original lesion. Animal 3 showed complete closure of the lesion but a visible skin covered depression remained.

The results clearly show that stem cell mediated tissue repair and regeneration can be dramatically improved by co-injecting embryo derived RNA fractions with the stem cells. It is clear, from this example and other similar studies by the present inventors, that the embryo RNA alters the host tissue environment around the tissue to signal injected stem cells to the damaged area. Further, the established scarring of the injury was similarly altered to provide a permissive environment for stem cell infiltration and subsequent repair of the lesion. With such co-treatment, stem cells are recruited to the damaged tissues and can reverse the damage once in location by regeneration of the relevant tissue types. Of great significance is the fact that the damage model used in this example is an old well established injury which stem cell injection alone cannot repair. This method provides a novel method of improving the efficacy of any potential stem cell therapy. Similar results have also been found using RNA extracted from foetal tissue maintained in tissue culture and injected up to 48 hours prior to stem cell injection. Longer intervals have not yet been investigated. Similarly, simultaneous injection of the RNA with stem cells achieves a similar major regeneration of damaged tissue. It is postulated that the embryo RNA re-creates the permissive regenerative environment and signalling environment of the foetal period.

### EXAMPLE 11

### In-Vivo Injection of Muscle RNA from Exercise Tolerant Rats Induces Exercise Tolerance in Sedentary Rats

Exercise is known to be beneficial to muscle anatomy and physiology. During repeated exercise micro damage to skeletal muscle induces both stem cell activity and changes in muscle cell biology. Such changes facilitate an increased tolerance for exercise with practice.

RNA extracted from hind limb muscles from exercised rats was injected to sedentary animals to investigate the effects of such treatment on recipient animal performance during heavy exercise. The exercise task involved running on a revolving drum. Rats readily learned to stay on the apparatus by running at an appropriate speed dictated by the revolution speed of the drum. As the animal tires and stops running it falls into a plastic bin filled with shredded paper. Once running skill had been perfected, animals would happily run on the apparatus until exhaustion. After a period of initial training on the apparatus, run time was recorded as a measure of exercise tolerance.

Experimental Donor rats (n=10) were trained daily on a suitable exercise regimen as follows:
Week 1—Animals were given 5 trials per day (10 minutes) with inter-trial interval of 1 hour. The revolution speed was set at 15 mm/sec. If the animal fell, it was placed back on the drum for the full duration of the trial. All animals mastered this motor skill readily over this orientation week.
Week 2—Animals were given 5 trials per day with an increased speed of 37 mm/sec with a 1-hour inter trial interval. If an animal fell, it was immediately placed back on the apparatus. Each trial was of 15 minutes duration.
Week 3—Animals were given 1 trial per day at the same run speed but run until the first fall.
Week 4—Animals were given 1 trial per day to first fall criterion at a run speed of 97 mm/sec.

Control Donor rats (n=10) were not exposed to the exercise apparatus and remained in their home cage throughout the 4-week exercise period.

Both groups of donors were sacrificed at the end of week 4 and hind limb muscles dissected. RNA was extracted by the method outlined in Example 1. RNA was then stored in 900 μg doses ready for injection.

Recipient animals (n=20) were divided into two matched groups. All recipient animals received an orientation week of training on the apparatus as described in donor week 1 training. They received no further conditioning.

One day after last orientation trial recipient rats received 900 μg of RNA dissolved in 0.3 ml of PBS (IV) into the tail vein. Experimental recipients received exercised muscle RNA, control animals received un-exercised RNA.

One-week post injection all rats received a run trial as follows: 5 minutes gentle running at 15 mm/sec. All rats balanced and ran comfortably in this session. After five minutes balance trial, the speed was increased to 97 mm/sec and the duration to falling off/jumping off was recorded as a measure of exercise tolerance.

There was a clear difference between the two groups. Recipients of non-exercised RNA showed a mean exercise time of 3.54 minutes. Recipients of muscle RNA from exercised rats showed a mean exercise time of 6.19 minutes.

The RNA extracted from the exercised animals enhanced exercise tolerance in recipient animals compared to controls. These preliminary data suggest that RNA may transfer exercise induced muscle enhancement to naïve muscle via in vivo application. This may provide a valuable therapeutic approach to various muscle degenerative diseases or a novel method to improve muscle mass in disease, ageing or age related pathology.

### EXAMPLE 12

### Effect of Polya Positive and Polya Negative RNA on the In Vitro Differentiation of Stem Cells

An effect has been seen with the addition of whole, unfractionated, RNA to cell cultures, with the result of the cells differentiating into cells of the type the RNA was extracted from. The following example illustrates that the polyA positive RNA fraction is the active fraction for cell differentiation.

Isolation of brain RNA. Sixteen P26 rats were sacrificed and their brains removed, placed in RNAlater™ (Ambion cat #7021) and stored on ice before incubation at 4° C. After 24 hours, the sample was removed from the RNAlater™ and placed in a SPEX CertiPrep 6850 Freezer Mill for milling under liquid nitrogen. The programme of sample preparation was 2 minutes pre-cooling, 1 minute milling, 1 minute cooling, 1 minute milling. The resulting powder was processed using an acid guanidinium thiocyanate-phenol-chloroform RNA extraction procedure (Chomczynski et al (1987) Analytical Chemistry 162,156-159).

The tissue samples were then reconstituted in solution D (0.36 ml 2-mercaptoethanol in 50 ml Solution A (250 g guanidinium thiocyanate in 293 ml distilled water with 17.6 ml sodium citrate (0.75M, pH 7) and 26.4 ml sarcosyl (10%))).

The volume of solution D added was in a ratio of 1 ml: 0.2 g tissue. The resultant mixture was triturated using a 10 ml syringe with a 19 gauge needle. After five triturations, 10% of the volume of sodium acetate (2M, pH 4.0) was added and mixed by inversion. An equal volume of phenol chloroform-iso-amyl alcohol (in a ratio of 25:24:1) as the volume of solution D used was added the resultant mixture shaken vigorously for 10 seconds before cooling at −20° C. for 15 min. After this time, the solution was transferred to 2 ml tubes, with a maximum of 1.2 ml in each, before centrifugation at 10,000 g for 20 minutes at 4° C.

Following centrifugation, the upper (aqueous) phase was transferred to a fresh 2 ml tube, to a maximum of 0.5 ml, and 1 ml isopropanol added. This mixture was then incubated at −20° C. for 15 minutes to precipitate RNA, before centrifugation at 14,000 rpm for 20 minutes at 4° C. An RNA pellet was obtained at the base of the tube. The supernatant was removed and 1.5 ml 100% ethanol added to the pellet. The mixture was vortexed and incubated at −80° C. for 30 min. The mixture was then centrifuged at 12,000 g for 20 minutes at 4° C. The supernatant was again removed and the pellet washed with 1 ml 70% ethanol, vortexed and centrifuged at 12,000 g for 10 minutes at 4° C. The resultant pellet was stored at −20° C. until required.

At this stage, the purity and concentration of the RNA sample produced were ascertained using a Genequant spectrophotometer. The average purity (A260/A280) was 1.82 and the average concentration was 548.69 μg/ml.

The total RNA was then further purified by the addition of 0.1 volume 3M sodium acetate, 1 μl glycogen and 2.5 volumes of 100% ethanol and the resultant mixture incubated at −70° C. for 30 minutes before centrifugation at 12,000 g for 25 minutes at 4° C. The supernatant was removed by aspiration and the pellet centrifuged once more at 12,000 g for 5 minutes at 4° C. to remove any remaining supernatant. 1 cm³ 70% ethanol was added, the mixture vortexed, and the RNA repelleted by centrifuging at 12,000 g for 10 minutes at 4° C. The supernatant was then removed.

Some of the resultant RNA pellet was set aside for use as total RNA later. For the remaining total RNA, samples of not more than 2,000 were resuspended in 0.75 cm³ nuclease free water and vortexed. An equal volume of 2× binding solution (Poly(A) Purist™ mRNA purification kit, manufacturer's protocol) was added and mixed thoroughly. Each RNA sample was then added to a tube containing 100 mg oligo(dT) cellulose and mixed by inversion. The resultant mixture was then heated to 70° C. in a water bath for 5 minutes. After this time, the mixture was agitated gently for 60 minutes at room temperature. The oligo(dT) cellulose was pelleted by centrifuging the mixture at 3000 g for 3 minutes at room temperature.

Isolation of polyA negative RNA fraction. The resultant supernatant (which contains the polyA negative RNA) was removed by aspiration and diluted by the addition of three volumes of nuclease free water and 0.1 volumes of 3M sodium acetate. Three volumes of 100% ethanol were then added and the mixture mixed thoroughly before chilling to −70° C. for 30 minutes. The mixture was then centrifuged at 12,000 g for 20 minutes at 4° C. The supernatant was removed by aspiration and the pellet washed by vortexing in 1 ml 70% ethanol. The resultant suspension was centrifuged at 12,000 g for 10 minutes at 4° C., leaving a polyA negative RNA pellet. This was stored at −20° C. until required.

Isolation of polyA positive RNA fraction. Separately, 0.5 cm³ of Wash Solution 1 (Poly(A) Purist™ mRNA purification kit, manufacturer's protocol) was added to the oligo(dT) cellulose pellet (which contains the polyA positive RNA) and the mixture vortexed to resuspend the pellet. A spin column was placed in a 2 ml microfuge tube and the oligo(dT) cellulose suspension transferred to this column, which was then centrifuged at 3000 g for 3 minutes at room temperature. The filtrate was discarded from the microfuge tube and the spin column returned to the tube. This washing step was repeated a further time with Wash Solution 1 and a further three times with Wash Solution 2 (Poly(A) Purist™ mRNA purification kit, manufacturer's protocol).

The spin column was then placed in a fresh microfuge tube and 200 μl of warm THE RNA Storage Solution (Ambion cat #7001) (previously heated to 70° C. in a water bath) added to the oligo(dT) cellulose pellet. The mixture was vortexed to mix the two and the tube immediately centrifuged at 5,000 g for 2 minutes at room temperature. This addition of warm THE RNA Storage Solution was repeated a further two times.

The spin column was discarded and 40 μl 5M ammonium acetate, 1 μl glycogen and 1.1 ml 100% ethanol added to the filtrate. This mixture (which contains the polyA positive RNA) was then stored at −70° C. for 30 minutes.

To recover the polyA positive RNA, the mixture was centrifuged at 12,000 g for 30 minutes at 4° C. and the supernatant removed by aspiration and discarded. The remaining pellet was then washed with 70% ethanol and vortexed. Finally, a polyA positive RNA pellet was obtained by centrifuging the resultant mixture at 12,000 g for 10 minutes at 4° C. This sample was stored at −20° C. until required.

Addition of total brain RNA, polyA positive brain RNA and polyA negative brain RNA to bone marrow cell cultures. Bone marrow cell cultures were cultured from 5 week-old rats, and the cultures grown in 75 cm² flasks for one month, going through one cell passage. The cells were confluent prior to addition of RNA.

The RNA samples were resuspended (after evaporation of residual ethanol) into a-MEMS media (Invitrogen cat #32571-093), supplemented with 10% foetal calf serum (Invitrogen cat #10108-165) and 3% penicillin/streptomycin (Invitrogen cat #15070-063). The media was removed from the cell culture flasks, and the total RNA sample applied directly to the cells. This was repeated with the polyA positive and polyA negative RNA samples. Furthermore, fresh a-MEMS was added as a control. The amount of RNA added was calculated to be 191.8 μg/ml.

After 24 hours, the cell culture media was changed. The cells were monitored daily for 6 days with photographs being taken. The cells were then passaged onto 6 well plates and photographed daily.

The cells treated with total RNA were not viable, with clumps of cells floating in the media. However, there was evidence of dendritic branching, and neurites (but not glia) were present.

The cells treated with polyA positive RNA showed signs of differentiation, with neurites, oligodendroglia and astroglia being present. The cells exhibited large projections and the differentiation survived passage.

In contrast, the cells treated with polyA negative RNA showed a lesser degree of differentiation than seen with either total RNA or polyA positive RNA. The differentiation included the presence of neurites, but not glia.

The control cells showed normal bone marrow cell growth. No signs of differentiation and no neurites or glia were seen.

These results show that total RNA and polyA positive RNA can induce a stable change in recipient stem cell differentiation. In contrast, polyA negative RNA can only induce a slight change in recipient stem cell differentiation, with this change being thought to result from a small amount of residual polyA positive RNA in the fraction.

It will be understood that the invention is described above by way of example only and modifications may be made whilst remaining within the scope and spirit of the invention.

### REFERENCES

Aviv et al. (1972) PNAS 69, 1408-1412
Bagnall, L. & Ray, S. (1999) Rat strain Differences on performance in the Morris water maze. Animal Technology. Vol. 50 (2). 69-77.
Bain, G et al., (1995) Dev. Biol. 168. 342-357
Chenchik, A., et al. (1998) Generation and use of high-quality cDNA from small amounts of total RNA by SMART PCR. In Gene Cloning and Analysis by RT-PCR (BioTechniques Books, MA), pp. 305-319.
Dai et al. (2000) Biol Blood Marrow Transplant. 6(4). 395-407.
Fandrich, F., Lin, X., Chai, G. X. et al. (2002) Preimplantation-stage stem cells induce long term allogeneic graft acceptance without supplementary host conditioning. Nature Medicine. Vol. 8. 171-178.
Felgner et al. (1987) Proc. Nat. Acad. Aci. Vol 84. 7413-7417.
Freshney, R. I. (2000). Culture of animal cells. Wiley-Liss NY
Gennaro (2000) Remington: The Science and Practice of Pharmacy. 20th edition, ISBN: 0683306472
Graham & van der Eb (1978) Virology 52:456-457.
Harrison, M A. & Rae, I F. (1997) General Techniques of Cell Culture. Cambridge Univ. Press. Cambridge.
Heus et al. (1990) Nucleic Acids Res. 18: 1103-1108
Homes and Korsnes (1990) Genet. Anal. Tech. Appl. 7: 145-150, and Jarret (1993) J. Chromatogr. 618: 315-339.
Jain et al. (1997) Mol. Cell. Biology 17(2): 954-962.
Kawaja, et al. (1992) In Neural Transplantation: A practical Approach. IRL Press. Oxford.
Keown et al. (1990) Methods in Enzymology 185:527-537
Kirby, K. S. (1956) J. Biochem. Vol. 64. 405.
Kirshenbaum et al. (1999) Curr Opin Struct Biol 9:530-5.
Lee & Marshall (1986) High-speed preparative-scale separation and purification of ribosomal 5S and 5.8S RNA's via Sephacryl S-300 gel filtration chromatography. Prep. Biochem. 16(3): 247-58
Mansour et al. (1988) Nature 336:348-352 (1988)
Owen, M. & Friedenstein, A J. (1988) Stromal stem cells: marrow derived osteogenic precursors CIBA Foundation Symposium 136. 42-60.
Pley et al. (1993) J. Biol. Chem. 268: 19656-19658.
Reynolds, B. A. & Weiss, S. (1992) Science. Vol. 255. 1707-1710.
Ruan et al. (1997) in Analysis of mRNA Formation and function, ed. Richter, J. D. (Academic, New York), pp. 305-321.
Ruhnke, M., Ungefroren, H., Zehle, G., Bader, M., Kremer, B., & Fandrich, F. (2003) Long-term culture and differentiation of rat embryonic stem cell-like cells into neuronal, glial, endothelial and hepatic lineages. Stem Cells. Vol. 21. (4). 428-436.
Sambrook and Russell (2001) Molecular Cloning. A Laboratory Manual (3^{rd} ed.) Cold Spring Harbor Laboratory Press, Cold Spring Harbor, N.Y.
Saneto, R. P. & de Vellis, J. (1987) Neuronal and Glial cells: cell culture of the central nervous system. In Turner, A. J. & Bachelard, H. S. (Eds.) Neurochemistry: A practical approach. IRL Press. Oxford.
Schnapp et al. (1998) One-step affinity purification protocol for human telomerase. Nucleic Acids Res. 26(13): 3311-3313.
Stewart, C. A. & Morris, R. G. M. (1993) The watermaze. In Sahgal. A (Ed.) Behavioural Neuroscience. A practical approach. Vol. 1. IRL Press. Oxford.
Tada, et al. (2001) Curr Biol. Vol 11(19). 1553-8.
Zong et al. (1999) Messenger RNA translation state: The second dimension of high-throughput expression screening. PNAS 96: 10632-10636.

1. A method of effecting a treatment response in a target tissue of a subject, comprising:
administering to the subject isolated RNA comprising an RNA sequence extractable or extracted from a source tissue such that said treatment response is effected;
wherein the RNA is isolated polyA positive RNA in substantially pure form.

2. A method of effecting a treatment response in a target tissue of a subject, comprising:
administering to the subject stem cells such that said treatment response is effected, wherein said stem cells have been treated with isolated RNA comprising an RNA sequence extractable from a source tissue;
wherein the RNA is isolated polyA positiveRNA in substantially pure form.

3. A method of effecting a treatment response in a target tissue of a subject, comprising:
i) administering to the subject stem cells; and
ii) administering to the subject isolated RNA comprising an RNA sequence extractable from a source tissue;
wherein the RNA is isolated polyA positive RNA in substantially pure form.

4. The method of claim 3 wherein said stem cells have been treated with isolated RNA comprising an RNA sequence extractable from a source tissue, wherein the RNA is isolated polyA positive RNA in substantially pure form.

5. The method according to any one of the preceding claims, wherein the treatment response is growth, regeneration, renewal or repair in the target tissue.

6. The method according to any one of claims 1 to 4, wherein the method is combined with a conventional therapy for promoting growth in the target tissue.

7. The method according to any one of claims 1 to 4, wherein the method is combined with a surgical therapy known to result in damage to the target tissue.

8. A method for grafting genetically modified cells into a target tissue of a subject comprising the steps of:
i) administering to the subject genetically modified stem cells to repopulate the bone marrow of the subject; and
ii) administering to the subject isolated RNA comprising an RNA sequence extractable from a source tissue such that engraftment of the cells within a target tissue is effected;
wherein the RNA is isolated polyA positive RNA in substantially pure form.

9. A method for grafting genetically modified cells into a target tissue of a subject comprising the step of:
administering to the subject genetically modified stem cells such that engraftment of the cells within a target tissue is effected; wherein said stem cells have been treated with isolated RNA comprising an RNA sequence extractable from a source tissue;
wherein the RNA is isolated polyA positive RNA in substantially pure form.

10. The method of claim 9, further comprising administering to the subject isolated RNA comprising an RNA sequence extractable from a source tissue, wherein the RNA is isolated polyA positive RNA in substantially pure form.

11. The method according of claim 9, further comprising administering genetically modified stem cells to repopulate the bone marrow of the subject.

12. The method according to any one of claims 1 to 4 or 8 to 11, wherein the subject is an adult.

13. The method according to any one of claims 1 to 4 or 8 to 11, wherein the target tissue does not comprise tumorigenic cells.

14. The method according to any one of claims 1 to 4 or 8 to 11, wherein any stem cells are autologous stem cells.
